(19) United States
(12) Patent Application Publication
(10) Pub. No.: US 2021/0340197 A1
(43) Pub. Date: Nov. 4, 2021

(54) DIRECTED PSEUDOURIDYLATION OF RNA

(71) Applicant: The Regents of the University of California, La Jolla, CA (US)

(72) Inventors: Eugene Yeo, La Jolla, CA (US); Kristopher Brannan, La Jolla, CA (US)

(21) Appl. No.: 17/272,018

(22) PCT Filed: Aug. 30, 2019
(86) PCT No.: PCT/US2019/049182

§ 371 Date: Feb. 26, 2021

Related U.S. Application Data
(60) Provisional application No. 62/726,149, filed on Aug. 31, 2018.

(51) Int. Cl. C07K 14/47; C12N 9/22; C12N 15/11

(52) U.S. Cl. CPC C07K 14/47; C12N 9/22; A61K 48/00; C12N 2800/80; C07K 2319/00; C12N 15/11

(57) ABSTRACT

Described herein are compositions, systems, methods, and kits utilizing CRISPR-Cas protein fusions comprising a guide nucleotide sequence-programmable RNA binding protein and a RNA pseudouridylation modification protein. The compositions, systems, methods, and kits described herein are useful to modulate RNA pseudouridylation.

### CROSS-REFERENCE TO RELATED APPLICATIONS

This application claims priority to: U.S. Patent Application Ser. No. 62/726,149, filed Aug. 31, 2018, which is incorporated hereby reference in its entirety.

### STATEMENT OF GOVERNMENT SUPPORT

This invention was made with government support under the HG004659, awarded by the National Institute for Health Research. The government have certain rights to the invention.

### BACKGROUND

Present strategies aimed to target and manipulate RNA in living cells mainly rely on the use of antisense oligonucleotides (ASO) or engineered RNA binding proteins (RBP). Although ASO therapies have been shown great promise in eliminating pathogenic transcripts or modulating RBP binding, they are synthetic in construction and thus cannot be encoded within DNA. This complicates potential gene therapy strategies, which would rely on regular administration of ASOs throughout the lifetime of the patient. Furthermore, they are incapable of modulating the genetic sequence of RNA. Although engineered RBPs such as PUF proteins can be designed to recognize target transcripts and fused to RNA modifying effectors to allow for specific recognition and manipulation, these constructs require extensive protein engineering for each target and may prove to be laborious and costly. Current systems used to directly pseudouridylate RNA rely on recruitment of endogenous pseudouridylation machinery by exogenously expressed guide RNAs, and have not yet been demonstrated to be effective in mammalian systems.

Accordingly, there is a need in the art for new methods of modulating RNA that can be simply and rapidly programed for specific mRNA targets. This disclosure satisfies this need and provides related advantages.

### SUMMARY

Described herein is are compositions, systems, methods, and kits to modulate RNA pseudouridylation using CRISPR-Cas protein fusions. These compositions, methods, systems, and kits utilize the RNA targeting abilities of CRISPR-Cas systems, which use a guide RNA to provide a simple and rapidly programmable system for recognizing RNA molecules in cells. CRISPR-Cas systems also have neutral effects on messenger RNA stability, which makes any measured change to protein expression a function of the fused protein effector. The compositions, systems, methods, and kits described herein provide high utility and versatility when compared to other compositions, methods, systems, and kits for modulating mRNA.

Accordingly, in some aspects, provided herein are fusion proteins comprising, consisting of, or consisting essentially of: (i) a guide nucleotide sequence-programmable RNA binding protein; and (ii) a RNA pseudouridylation modification protein (RPMP), or an equivalent thereof.

In some embodiments, the guide nucleotide sequence-programmable RNA binding protein is selected from: Cas9, modified Cas9, Cas13a, Cas13b, CasRX/Cas13d, and a biological equivalent of each thereof. In some embodiments, the guide nucleotide sequence-programmable RNA binding protein is selected from: Steptococcus pyogenes Cas9 (spCas9), Staphylococcus aureus Cas9 (saCas9), Francisella novicida Cas9 (FnCas9), Neisseria meningitidis Cas9 (nmCas9), Streptococcus thermophilus 1 Cas9 (St1Cas9), Streptococcus thermophilus 3 Cas9 (St3Cas9), Campylobacter jejuni Cas9 (CjeCas9), and Brevibacillus laterosporus Cas9 (BlatCas9). In some embodiments, the guide nucleotide sequence-programmable RNA binding protein is nuclease inactive.

In some embodiments, the fusion peptide further comprises, consists of, or consists essentially of a linker. In some embodiments, the linker is a peptide linker. In some embodiments, the peptide linker further comprises, consists of, or consists essentially of an XTEN linker or one or more repeats of the tri-peptide GGS. In some embodiments, the linker is a non-peptide linker. In some embodiments, the non-peptide linker comprises polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker.

In some embodiments, the fusion protein comprises the structure NH₂-[RPMP]-[linker]-[guide nucleotide sequence-programmable RNA binding protein]-COOH. In other embodiments, the fusion protein comprises the structure NH₂-[guide nucleotide sequence-programmable RNA binding protein]-[linker]-[RPMP]-COOH.

In some embodiments, the guide nucleotide sequence-programmable RNA binding protein is bound to a guide RNA (gRNA), a crisprRNA (crRNA), or a trans-activating crRNA (tracrRNA).

In some embodiments, the RPMP protein is selected from H/ACA ribonucleoprotein complex subunit 4 (DKC1), tRNA pseudouridine synthase A (PUS1), tRNA pseudouridylate synthase 3 (PUS3), pseudouridylate synthase 7 (PUS7), pseudouridylate synthase 7 like (PUSL), and a biological equivalent of each thereof. In some embodiments, the RPMP protein has an nucleotide sequence comprising, consisting of, or consisting essentially of all or part of a sequence selected from NM_001142463, NM_001288747, NM_001363, NM_001002019, NM_001002020, NM_025215, NM_031307, NM_001271985, NM_019042, NM_001318164, NM_001318163, NM_001098614, NM_001098615, NM_001271826, NM_031292, and a biological equivalent of each thereof.

In some aspects, provided herein is a polynucleotide encoding a fusion protein comprising, consisting of, or consisting essentially of: (i) a guide nucleotide sequence-programmable RNA binding protein; and (ii) a RNA pseudouridylation modification protein (RPMP), or an equivalent thereof.

In some embodiments, provided herein are polynucleotides encoding a guide RNA or a crRNA comprising, consisting of, or consisting essentially of a sequence complementary to a target RNA. In some embodiments, the target RNA is an mRNA. In some embodiments, the target RNA comprises, consists of, or consists essentially of a premature stop codon. In some embodiments, the target RNA is susceptible to nonsense mediated decay. In some embodiments, the gRNA or the crRNA comprises, consists of, or consists essentially of a nucleotide sequence complementary to a target RNA with a mismatch at a uridine residue. In some embodiments, the gRNA or the crRNA further comprises, consists of, or consists essentially of a nucleotide sequence that mimics a hairpin-hinge-hairpin-tail conformation. In some embodiments, the gRNA contains a guide pocket tract that specifies a pseudouridylation target.

In some aspects, provided herein is a vector comprising a polynucleotide encoding a fusion protein comprising, consisting of, or consisting essentially of: (i) a guide nucleotide sequence-programmable RNA binding protein; and (ii) a RNA pseudouridylation modification protein (RPMP), or an equivalent thereof, optionally wherein the vector is an adenoviral vector, an adeno-associated viral vector, or a lentiviral vector. In some embodiments, the vector further comprises an expression control element. In some embodiments, the vector further comprises, consists of, or consists essentially of a selectable marker. In some embodiments, the vector further comprises, consists of, or consists essentially of a polynucleotide encoding either (i) a gRNA, or (ii) a crRNA and a tracrRNA. In some embodiments, the gRNA or the crRNA comprises a nucleotide sequence complementary to a target RNA.

In some aspects, provided herein is a viral particle that comprises, consists of, or consists essentially of a vector comprising a polynucleotide encoding a fusion protein comprising, consisting of, or consisting essentially of: (i) a guide nucleotide sequence-programmable RNA binding protein; and (ii) a RNA pseudouridylation modification protein (RPMP), or an equivalent thereof.

In some aspects, provided herein is a cell comprising, consisting of, or consisting essentially of a fusion protein, a polynucleotide, a vector, or a viral particle as described herein. In some embodiments, the cell is a eukaryotic cell. In some embodiments, the the cell is a prokaryotic cell. In some embodiments, the cell is a mammalian cell, optionally a bovine, murine, feline, equine, porcine, canine, simian, or human cell.

In some aspects, provided herein is a system for modulating RNA pseudouridylation of a target RNA, the system comprising, consisting of, or consisting essentially of: (a) a fusion protein comprising, consisting of, or consisting essentially of: (i) a guide nucleotide sequence-programmable RNA binding protein; and (ii) a RNA pseudouridylation modification protein (RPMP), or an equivalent thereof and (b) a gRNA; or (c) a crRNA and a tracrRNA; wherein the gRNA or the crRNA comprises, consists of, or consists essentially of a sequence complementary to a target RNA. In some embodiments, the system further comprises, consists of, or consists essentially of a PAMmer. In some embodiments, the target RNA does not comprise a PAM sequence or complement thereof.

In some aspects, provided herein is a method for modulating RNA pseudouridylation of a target RNA, the method comprising, consisting of, or consisting essentially of contacting the target mRNA with a fusion protein comprising, consisting of, or consisting essentially of: (i) a guide nucleotide sequence-programmable RNA binding protein; and (ii) a RNA pseudouridylation modification protein (RPMP), wherein the guide nucleotide sequence-programmable RNA binding protein binds a gRNA or a crRNA that hybridizes to a region of the target RNA.

In some aspects, provided herein is a method for modulating embryonic stem cell maintenance and/or differentiation, nervous system development, circadian rhythm, heat shock response, meiotic progression, DNA ultraviolet (UV) damage response, or XIST mediated gene silencing, the method comprising, consisting of, or consisting essentially of contacting a target mRNA with a fusion protein comprising, consisting of, or consisting essentially of: (i) a guide nucleotide sequence-programmable RNA binding protein; and (ii) a RNA pseudouridylation modification protein (RPMP), or an equivalent thereof, wherein the guide nucleotide sequence-programmable RNA binding protein binds a gRNA or a crRNA that hybridizes to a region of the target RNA. In some embodiments, the target mRNA comprises, consists of, or consists essentially of a PAM sequence or complement thereof. In some embodiments, the target mRNA does not comprise a PAM sequence or complement thereof. In some embodiments, the target mRNA is in a cell. In some embodiments, the cell is a eukaryotic cell. In some embodiments, the cell is a prokaryotic cell. In some embodiments, the eukaryotic cell is a mammalian cell, optionally a bovine, murine, feline, equine, porcine, canine, simian, or human cell. In some embodiments, the cell is in a subject.

In some aspects, provided herein is a method for treating a disease or condition associated with RNA pseudouridylation of a target RNA in a subject in need thereof, the method comprising, consisting of, or consisting essentially of administering a fusion protein, polynucleotide, vector, viral particle, and/or cell as described herein to the subject, thereby treating the disease or condition associated with RNA pseudouridylation. In some embodiments, the disease or condition associated with RNA pseudouridylation is selected from cancer, growth retardation, developmental delay, facial dysmorphism, Alzheimer's disease, diabetes, and major depressive disorder. In some embodiments, the subject is a human. In some embodiments, the methods further comprise administering to the subject: (i) a gRNA complementary to the target RNA, or (ii) a crRNA complementary to the target RNA and a tracrRNA. In some embodiments, the methods further comprise administering a PAMmer to the subject.

In some aspects, provided herein is a kit comprising, consisting of, or consisting essentially of one or more of: a fusion protein, polynucleotide, vector, viral particle, and/or cell as described herein; and optionally instructions for use. In some embodiments, the kit further comprises, consists of, or consists essentially of one or more nucleic acids selected from: (i) a gRNA; (ii) a crRNA and a tracrRNA; (iii) a PAMmer; and (iv) a vector for expressing the nucleic acid of (i), (ii), and/or (iii).

In some aspects, provided herein is a non-human transgenic animal comprising, consisting of, or consisting essentially of a fusion protein or viral vector as described herein.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

The practice of the present technology will employ, unless otherwise indicated, conventional techniques of tissue culture, immunology, molecular biology, microbiology, cell biology, and recombinant DNA, which are within the skill of the art.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

### Definitions

As used in the description of the invention and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11 or 12, sequentially numbered, are disclosed in the prior art. Non-limiting exemplary serotypes useful in the methods disclosed herein include any of the 11 or 12 serotypes, e.g., AAV2, AAV5, and AAV8, or variant serotypes, e.g. AAV-DJ. The AAV structural particle is composed of 60 protein molecules made up of VP1, VP2 and VP3. Each particle contains approximately 5 VP1 proteins, 5 VP2 proteins and 50 VP3 proteins ordered into an icosahedral structure.

Also as used herein, “and/or” refers to and encompasses any and all possible combinations of one or more of the associated listed items, as well as the lack of combinations when interpreted in the alternative (“or”).

The term “guide nucleotide sequence-programmable RNA binding protein” refers to a CRISPR-associated, RNA-guided endonuclease such as Streptococcus pyogenes Cas9 (spCas9) and orthologs and biological equivalents thereof. Biological equivalents of Cas9 include but are not limited to Type VI CRISPR systems, such as Cas13a, C2c2, and Cas13b, which target RNA rather than DNA. A guide nucleotide sequence-programmable RNA binding protein may refer to an endonuclease that causes breaks or nicks in RNA as well as other variations such as dead Cas9 or dCas9, which lack endonuclease activity. A guide nucleotide sequence-programmable RNA binding protein may also refer to a “split” protein in which the protein is split into two halves (e.g., C-Cas9 and N-Cas9) and fused with two intein moieties. See, e.g., U.S. Pat. No. 9,074,199 B1; Zetsche et al. (2015) Nat Biotechnol. 33(2):139-42; Wright et al. (2015) PNAS 112(10) 2984-89.

In particular embodiments, the guide nucleotide sequence-programmable RNA binding protein is modified to eliminate endonuclease activity (“nuclease dead”). For example, both RuvC and HNH nuclease domains can be rendered inactive by point mutations (e.g., D10A and H840A in SpCas9), resulting in a nuclease dead Cas9 (dCas9) molecule that cannot cleave target DNA. The dCas9 molecule retains the ability to bind to target RNA based on the gRNA targeting sequence.

Further nonlimiting examples of orthologs and biological equivalents Cas9 are provided in the table below:

| Name | Protein Sequence |
|---|---|
| S. pyogenes Cas9 | MDKKYSIGLDIGTNSVGWAVITDEYKVPSKKFKVLGNTDRHSIK |
| | KNLIGALLFDSGETAEATRLKRTARRRYTRRKNRICYLQEIFSNE |
| | MAKVDDSFFHRLEESFLVEEDKKHERHPIFGNIVDEVAYHEKYPT |
| | IYHLRKKLVDSTDKADLRLIYLALAHMIKFRGHFLIEGDLNPDNS |
| | DVDKLFIQLVQTYNQLFEENPINASGVDAKAILSARLSKSRRLENL |
| | IAQLPGEKKNGLFGNLIALSLGLTPNFKSNFDLAEDAKLQLSKDT |
| | YDDDLDNLLAQIGDQYADLFLAAKNLSDAILLSDILRVNTEITKA |
| | PLSASMIKRYDEHHQDLTLLKALVRQQLPEKYKEIFFDQSKNGYA |
| | GYIDGGASQEEFYKFIKPILEKMDGTEELLVKLNREDLLRKQRTF |
| | DNGSIPHQIHLGELHAILRRQEDFYPFLKDNREKIEKILTFRIPYYV |
| | GPLARGNSRFAWMTRKSEETITPWNFEEVVDKGASAQSFIERMT |
| | NFDKNLPNEKVLPKHSLLYEYFTVYNELTKVKYVTEGMRKPAFL |
| | SGEQKKAIVDLLFKTNRKVTVKQLKEDYFKKIECFDSVEISGVED |
| | RFNASLGTYHDLLKIIKDKDFLDNEENEDILEDIVLTLTLFEDREMI |
| | EERLKTYAHLFDDKVMKQLKRRRYTGWGRLSRKLINGIRDKQSG |
| | KTILDFLKSDGFANRNFMQLIHDDSLTFKEDIQKAQVSGQGDSLH |
| | EHIANLAGSPAIKKGILQTVKVVDELVKVMGRHKPENIVIEMARE |
| | NQTTQKGQKNSRERMKRIEEGIKELGSQILKEHPVENTQLQNEKL |
| | YLYYLQNGRDMYVDQELDINRLSDYDVDHIVPQSFLKDDSIDNK |
| | VLTRSDKNRGKSDNVPSEEVVKKMKNYWRQLLNAKLITQRKFD |
| | NLTKAERGGLSELDKAGFIKRQLVETRQITKHVAQILDSRMNTKY |
| | DENDKLIREVKVITLKSKLVSDFRKDFQFYKVREINNYHHAHDA |
| | YLNAVVGTALIKKYPKLESEFVYGDYKVYDVRKMIAKSEQEIGK |
| | ATAKYFFYSNIMNFFKTEITLANGEIRKRPLIETNGETGEIVWDKG |
| | RDFATVRKVLSMPQVNIVKKTEVQTGGFSKESILPKRNSDKLIAR |
| | KKDWDPKKYGGFDSPTVAYSVLVVAKVEKGKSKKLKSVKELLG |
| | ITIMERSSFEKNPIDFLEAKGYKEVKKDLIIKLPKYSLFELENGRKR |
| | MLASAGELQKGNELALPSKYVNFLYLASHYEKLKGSPEDNEQKQ |
| | LFVEQHKHYLDEIIEQISEFSKRVILADANLDKVLSAYNKHRDKPI |
| | REQAENIIHLFTLTNLGAPAAFKYFDTTIDRKRYTSTKEVLDATLI |
| | HQSITGLYETRIDLSQLGGD* |
| Staphylococcus | MKRNYILGLDIGITSVGYGIIDYETRDVIDAGVRLFKEANVENNE |
| aureus Cas9 | GRRSKRGARRLKRRRRHRIQRVKKLLFDYNLLTDHSELSGINPYE |
| | ARVKGLSQKLSEEEFSAALLHLAKRRGVHNVNEVEEDTGNELST |
| | KEQISRNSKALEEKYVAELQLERLKKDGEVRGSINRFKTSDYVKE |
| | AKQLLKVQKAYHQLDQSFIDTYIDLLETRRTYYEGPGEGSPFGW |
| | KDIKEWYEMLMGHCTYFPEELRSVKYAYNADLYNALNDLNNLV |
| | ITRDENEKLEYYEKFQIIENVFKQKKKPTLKQIAKEILVNEEDIKG |
| | YRVTSTGKPEFTNLKVYHDIKDITARKEIIENAELLDQIAKILTIYQ |
| | SSEDIQEELTNLNSELTQEEIEQISNLKGYTGTHNLSLKAINLILDE |
| | LWHTNDNQIAIFNRLKLVPKKVDLSQQKEIPTTLVDDFILSPVVKR |
| | SFIQSIKVINAIIKKYGLPNDIIIELAREKNSKDAQKMINEMQKRNR |
| | QTNERIEEIIRTTGKENAKYLIEKIKLHDMQEGKCLYSLEAIPLEDL |
| | LNNPFNYEVDHIIPRSVSFDNSFNNKVLVKQEENSKKGNRTPFQY |
| | LSSSDSKISYETFKKHILNLAKGKGRISKTKKEYLLEERDINRFSVQ |
| | KDFINRNLVDTRYATRGLMNLLRSYFRVNNLDVKVKSINGGFTS |
| | FLRRKWKFKKERNKGYKHHAEDALIIANADFIFKEWKKLDKAK |
| | KVMENQMFEEKQAESMPEIETEQEYKEIFITPHQIKHIKDFKDYK |
| | YSHRVDKKPNRELINDTLYSTRKDDKGNTLIVNNLNGLYDKDND |
| | KLKKLINKSPEKLLMYHHDPQTYQKLKLIMEQYGDEKNPLYKYY |
| | EETGNYLTKYSKKDNGPVIKKIKYYGNKLNAHLDITDDYPNSRN |
| | KVVKLSLKPYRFDVYLDNGVYKFVTVKNLDVIKKENYYEVNSK |
| | CYEEAKKLKKISNQAEFIASFYNNDLIKINGELYRVIGVNNDLLNR |
| | IEVNMIDITYREYLENMNDKRPPRIIKTIASKTQSIKKYSTDILGNL |
| | YEVKSKKHPQIIKKG* |
| S. thermophilus | MSDLVLGLDIGIGSVGVGILNKVTGEIIHKNSRIFPAAQAENNLVR |
| CRISPR 1 Cas9 | RTNRQGRRLARRKKHRRVRLNRLFEESGLITDFTKISINLNPYQLR |
| | VKGLTDELSNEELFIALKNMVKHRGISYLDDASDDGNSSVGDYA |
| | QIVKENSKQLETKTPGQIQLERYQTYGQLRGDFTVEKDGKKHRLI |
| | NVFPTSAYRSEALRILQTQQEFNPQITDEFINRYLEILTGKRKYYH |
| | GPGNEKSRTDYGRYRTSGETLDNIFGILIGKCTFYPDEFRAAKASY |
| | TAQEFNLLNDLNNLTVPTETKKLSKEQKNQIINYVKNEKAMGPA |
| | KLFKYIAKLLSCDVADIKGYRIDKSGKAEIHTFEAYRKMKTLETL |
| | DIEQMDRETLDKLAYVLTLNTEREGIQEALEHEFADGSFSQKQVD |
| | ELVQFRKANSSIFGKGWHNFSVKLMMELIPELYETSEEQMTILTR |
| | LGKQKTTSSSNKTKYIDEKLLTEEIYNPVVAKSVRQAIKIVNAAIK |
| | EYGDFDNIVIEMARETNEDDEKKAIQKIQKANKDEKDAAMLK |
| | AANQYNGKAELPHSVFHGHKQLATKIRLWHQQGERCLYTGKTIS |
| | IHDLINNSNQFEVDHILPLSITFDDSLANKVLVYATANQEKGQRTP |
| | YQALDSMDDAWSFRELKAFVRESKTLSNKKKEYLLTEEDISKFD |
| | VRKKFIERNLVDTRYASRVVLNALQEHFRAHKIDTKVSVVRGQF |
| | TSQLRRHWGIEKTRDTYHHHAVDALIIAASSQLNLWKKQKNTLV |
| | SYSEDQLLDIETGELISDDEYKESVFKAPYQHFVDTLKSKEFEDSI |
| | LFSYQVDSKFNRKISDATIYATRQAKVGKDKADETYVLGKIKDIY |
| | TQDGYDAFMKIYKKDKSKFLMYRHDPQTFEKVIEPILENYPNKQI |
| | NDKGKEVPCNPFLKYKEEHGYIRKYSKKGNGPEIKSLKYYDSKL |
| | GNHIDITPKDSNNKVVLQSVSPWRADVYFNKTTGKYEILGLKYA |
| | DLQFDKGTGTYKISQEKYNDIKKKEGVDSDSEFKFTLYKNDLLLV |
| | KDTETKEQQLFRFLSRTMPKQKHYVELKPYDKQKFEGGEALIKV |
| | LGNVANSGQCKKGLGKSNISIYKVRTDVLGNQHIIKNEGDKPKLD |
| | F* |
| N. meningitidis Cas9 | MAAFKPNPINYILGLDIGIASVGWAMVEIDEDENPICLIDLGVRVF |
| | ERAEVPKTGDSLAMARRLARSVRRLTRRRAHRLLRARRLLKREG |
| | VLQAADFDENGLIKSLPNTPWQLRAAALDRKLTPLEWSAVLLHLI |
| | KHRGYLSQRKNEGETADKELGALLKGVADNAHALQTGDFRTPA |
| | ELALNKFEKESGHIRNQRGDYSHTFSRKDLQAELILLFEKQKEFG |
| | NPHVSGGLKEGIETLLMTQRPALSGDAVQKMLGHCTFEPAEPKA |
| | AKNTYTAERFIWLTKLNNLRILEQGSERPLTDTERATLMDEPYRK |
| | SKLTYAQARKLLGLEDTAFFKGLRYGKDNAEASTLMEMKAYHA |
| | ISRALEKEGLKDKKSPLNLSPELQDEIGTAFSLFKTDEDITGRLKD |
| | RIQPEILEALLKHISFDKFVQISLKALRRIVPLMEQGKRYDEACAEI |
| | YGDHYGKKNTEEKIYLPPIPADEIRNPVVLRALSQARKVINGVVR |
| | RYGSPARIHIETAREVGKSFKDRKEIEKRQEENRKDREKAAAKFR |
| | EYFPNFVGEPKSKDILKLRLYEQQHGKCLYSGKEINLGRLNEKGY |
| | VEIDHALPFSRTWDDSFNNKVLVLGSENQNKGNQTPYEYFNGKD |
| | NSREWQEFKARVETSRFPRSKKQRILLQKFDEDGFKERNLNDTRY |
| | VNRFLCQFVADRMRLTGKGKKRVFASNGQITNLLRGFWGLRKV |
| | RAENDRHHALDAVVVACSTVAMQQKITRFVRYKEMNAFDGKTI |
| | DKETGEVLHQKTHFPQPWEFFAQEVMIRVFGKPDGKPEFEEADT |
| | PEKLRTLLAEKLSSRPEAVHEYVTPLFVSRAPNRKMSGQGHMET |
| | VKSAKRLDEGVSVLRVPLTQLKLKDLEKMVNREREPKLYEALKA |
| | RLEAHKDDPAKAFAEPFYKYDKAGNRTQQVKAVRVEQVQKTG |
| | VWVRNHNGIADNATMVRVDVFEKGDKYYLVPIYSWQVAKGILP |
| | DRAVVQGKDEEDWQLIDDSFNFKFSLHPNDLVEVITKKARMFGY |
| | FASCHRGTGNINIRIHDLDHKIGKNGILEGIGVKTALSFQKYQIDEL |
| | GKEIRPCRLKKRPPVR* |
| Parvibaculum | MERIFGFDIGTTSIGFSVIDYSSTQSAGNIQRLGVRIFPEARDPDGTP |
| lavamentivorans | LNQQRRQKRMMRRQLRRRRIRRKALNETLHEAGFLPAYGSADW |
| Cas9 | PVVMADEPYELRRRGLEEGLSAYEFGRAIYHLAQHRHFKGRELE |
| | ESDTPDPDVDDEKEAANERAATLKALKNEQTTLGAWLARRPPSD |
| | RKRGIHAHRNVVAEEFERLWEVQSKFHPALKSEEMRARISDTIFA |
| | QRPVFWRKNTLGECRFMPGEPLCPKGSWLSQQRRMLEKLNNLAI |
| | AGGNARPLDAEERDAILSKLQQQASMSWPGVRSALKALYKQRG |
| | EPGAEKSLKFNLELGGESKLLGNALEAKLADMFGPDWPAHPRKQ |
| | EIRHAVHERLWAADYGETPDKKRVIILSEKDRKAHREAAANSFV |
| | ADFGITGEQAAQLQALKLPTGWEPYSIPALNLFLAELEKGERFGA |
| | LVNGPDWEGWRRTNFPHRNQPTGEILDKLPSPASKEERERISQLR |
| | NPTVVRTQNELRKVVNNLIGLYGKPDRIRIEVGRDVGKSKREREE |
| | IQSGIRRNEKQRKKATEDLIKNGIANPSRDDVEKWILWKEGQERC |
| | PYTGDQIGFNALFREGRYEVEHIWPRSRSFDNSPRNKTLCRKDVN |
| | IEKGNRMPFEAFGHDEDRWSAIQIRLQGMVSAKGGTGMSPGKVK |
| | RFLAKTMPEDFAARQLNDTRYAAKQILAQLKRLWPDMGPEAPV |
| | KVEAVTGQVTAQLRKLWTLNNILADDGEKTRADHRHHAIDALT |
| | VACTHPGMTNKLSRYWQLRDDPRAEKPALTPPWDTIRADAEKA |
| | VSEIVVSHRVRKKVSGPLHKETTYGDTGTDIKTKSGTYRQFVTRK |
| | KIESLSKGELDEIRDPRIKEIVAAHVAGRGGDPKKAFPPYPCVSPG |
| | GPEIRKVRLTSKQQLNLMAQTGNGYADLGSNHHIAIYRLPDGKA |
| | DFEIVSLFDASRRLAQRNPIVQRTRADGASFVMSLAAGEAIMIPEG |
| | SKKGIWIVQGVWASGQVVLERDTDADHSTTTRPMPNPILKDDAK |
| | KVSIDPIGRVRPSND* |
| Corynebacter | MKYHVGIDVGTFSVGLAAIEVDDAGMPIKTLSLVSHIHDSGLDPD |
| diphtheria Cas9 | EIKSAVTRLASSGIARRTRRLYRRKRRRLQQLDKFIQRQGWPVIEL |
| | EDYSDPLYPWKVRAELAASYIADEKERGEKLSVALRHIARHRGW |
| | RNPYAKVSSLYLPDGPSDAFKAIREEIKRASGQPVPETATVGQMV |
| | TLCELGTLKLRGEGGVLSARLQQSDYAREIQEICRMQEIGQELYR |
| | KIIDVVFAAESPKGSASSRVGKDPLQPGKNRALKASDAFQRYRIA |
| | ALIGNLRVRVDGEKRILSVEEKNLVFDHLVNLTPKKEPEWVTIAEI |
| | LGIDRGQLIGTATMTDDGERAGARPPTHDTNRSIVNSRIAPLVDW |
| | WKTASALEQHAMVKALSNAEVDDFDSPEGAKVQAFFADLDDDV |
| | HAKLDSLHLPVGRAAYSEDTLVRLTRRMLSDGVDLYTARLQEFG |
| | IEPSWTPPTPRIGEPVGNPAVDRVLKTVSRWLESATKTWGAPERV |
| | IIEHVREGFVTEKRAREMDGDMRRRAARNAKLFQEMQEKLNVQ |
| | GKPSRADLWRYQSVQRQNCQCAYCGSPITFSNSEMDHIVPRAGQ |
| | GSTNTRENLVAVCHRCNQSKGNTPFAIWAKNTSIEGVSVKEAVE |
| | RTRHWVTDTGMRSTDFKKFTKAVVERFQRATMDEEIDARSMES |
| | VAWMANELRSRVAQHFASHGTTVRVYRGSLTAEARRASGISGK |
| | LKFFDGVGKSRLDRRHHAIDAAVIAFTSDYVAETLAVRSNLKQS |
| | QAHRQEAPQWREFTGKDAEHRAAWRVWCQKMEKLSALLTEDL |
| | RDDRVVVMSNVRLRLGNGSAHKETIGKLSKVKLSSQLSVSDIDK |
| | ASSEALWCALTREPGFDPKEGLPANPERHIRVNGTHVYAGDNIGL |
| | FPVSAGSIALRGGYAELGSSFHHARVYKITSGKKPAFAMLRVYTI |
| | DLLPYRNQDLFSVELKPQTMSMRQAEKKLRDALATGNAEYLGW |
| | LVVDDELVVDTSKIATDQVKAVEAELGTIRRWRVDGFFSPSKLRL |
| | RPLQMSKEGIKKESAPELSKIIDRPGWLPAVNKLFSDGNVTVVRR |
| | DSLGRVRLESTAHLPVTWKVQ* |
| Streptococcus | MTNGKILGLDIGIASVGVGIIEAKTGKVVHANSRLFSAANAENNA |
| pasteurtanus Cas9 | ERRGFRGSRRLNRRKKHRVKRVRDLFEKYGIVTDFRNLNLNPYE |
| | LRVKGLTEQLKNEELFAALRTISKRRGISYLDDAEDDSTGSTDYA |
| | KSIDENRRLLKNKTPGQIQLERLEKYGQLRGNFTVYDENGEAHRL |
| | INVFSTSDYEKEARKILETQADYNKKITAEFIDDYVEILTQKRKYY |
| | HGPGNEKSRTDYGRFRTDGTTLENIFGILIGKCNFYPDEYRASKAS |
| | YTAQEYNFLNDLNNLKVSTETGKLSTEQKESLVEFAKNTATLGP |
| | AKLLKEIAKILDCKVDEIKGYREDDKGKPDLHTFEPYRKLKFNLE |
| | SINIDDLSREVIDKLADILTLNTEREGIEDAIKRNLPNQFTEEQISEII |
| | KVRKSQSTAFNKGWHSFSAKLMNELIPELYATSDEQMTILTRLEK |
| | FKVNKKSSKNTKTIDEKEVTDEIYNPVVAKSVRQTIKIINAAVKK |
| | YGDFDKIVIEMPRDKNADDEKKFIDKRNKENKKEKDDALKRAA |
| | YLYNSSDKLPDEVFHGNKQLETKIRLWYQQGERCLYSGKPISIQE |
| | LVHNSNNFEIDHILPLSLSFDDSLANKVLVYAWTNQEKGQKTPYQ |
| | VIDSMDAAWSFREMKDYVLKQKGLGKKKRDYLLTTENIDKIEV |
| | KKKFIERNLVDTRYASRVVLNSLQSALRELGKDTKVSVVRGQFT |
| | SQLRRKWKIDKSRETYHHHAVDALIIAASSQLKLWEKQDNPMFV |
| | DYGKNQVVDKQTGEILSVSDDEYKELVFQPPYQGFVNTISSKGFE |
| | DEILFSYQVDSKYNRKVSDATIYSTRKAKIGKDKKEETYVLGKIK |
| | DIYSQNGFDTFIKKYNKDKTQFLMYQKDSLTWENVIEVILRDYPT |
| | TKKSEDGKNDVKCNPFEEYRRENGLICKYSKKGKGTPIKSLKYY |
| | DKKLGNCIDITPEESRNKVILQSINPWRADVYFNPETLKYELMGL |
| | KYSDLSFEKGTGNYHISQEKYDAIKEKEGIGKKSEFKFTLYRNDLI |
| | LIKDIASGEQEIYRFLSRTMPNVNHYVELKPYDKEKFDNVQELVE |
| | ALGEADKVGRCIKGLNKPNISIYKVRTDVLGNKYFVKKKGDKPK |
| | LDFKNNKK* |
| Neisseria cinerea | MAAFKPNPMNYILGLDIGIASVGWAIVEIDEEENPIRLIDLGVRVF |
| Cas9 | ERAEVPKTGDSLAAARRLARSVRRLTRRRAHRLLRARRLLKREG |
| | VLQAADFDENGLIKSLPNTPWQLRAAALDRKLTPLEWSAVLLHLI |
| | KHRGYLSQRKNEGETADKELGALLKGVADNTHALQTGDFRTPA |
| | ELALNKFEKESGHIRNQRGDYSHTFNRKDLQAELNLLFEKQKEFG |
| | NPHVSDGLKEGIETLLMTQRPALSGDAVQKMLGHCTFEPTEPKA |
| | AKNTYTAERFVWLTKLNNLRILEQGSERPLTDTERATLMDEPYR |
| | KSKLTYAQARKLLDLDDTAFFKGLRYGKDNAEASTLMEMKAYH |
| | AISRALEKEGLKDKKSPLNLSPELQDEIGTAFSLFKTDEDITGRLK |
| | DRVQPEILEALLKHISFDKFVQISLKALRRIVPLMEQGNRYDEACT |
| | EIYGDHYGKKNTEEKIYLPPIPADEIRNPVVLRALSQARKVINGVV |
| | RRYGSPARIHIETAREVGKSFKDRKEIEKRQEENRKDREKSAAKF |
| | REYFPNFVGEPKSKDILKLRLYEQQHGKCLYSGKEINLGRLNEKG |
| | YVEIDHALPFSRTWDDSFNNKVLALGSENQNKGNQTPYEYFNGK |
| | DNSREWQEFKARVETSRFPRSKKQRILLQKFDEDGFKERNLNDTR |
| | YINRFLCQFVADHMLLTGKGKRRVFASNGQITNLLRGFWGLRKV |
| | RAENDRHHALDAVVVACSTIAMQQKITRFVRYKEMNAFDGKTID |
| | KETGEVLHQKAHFPQPWEFFAQEVMIRVFGKPDGKPEFEEADTP |
| | EKLRTLLAEKLSSRPEAVHKYVTPLFISRAPNRKMSGQGHMETV |
| | KSAKRLDEGISVLRVPLTQLKLKDLEKMVNREREPKLYEALKAR |
| | LEAHKDDPAKAFAEPFYKYDKAGNRTQQVKAVRVEQVQKTGV |
| | WVHNHNGIADNATIVRVDVFEKGGKYYLVPIYSWQVAKGILPDR |
| | AVVQGKDEEDWTVMDDSFEFKFVLYANDLIKLTAKKNEFLGYF |
| | VSLNRATGAIDIRTHDTDSTKGKNGIFQSVGVKTALSFQKYQIDE |
| | LGKEIRPCRLKKRPPVR* |
| Campylobacter lari | MRILGFDIGINSIGWAFVENDELKDCGVRIFTKAENPKNKESLALP |
| Cas9 | RRNARSSRRRLKRRKARLIAIKRILAKELKLNYKDYVAADGELPK |
| | AYEGSLASVYELRYKALTQNLETKDLARVILHIAKHRGYMNKNE |
| | KKSNDAKKGKILSALKNNALKLENYQSVGEYFYKEFFQKYKKNT |
| | KNFIKIRNTKDNYNNCVLSSDLEKELKLILEKQKEFGYNYSEDFIN |
| | EILKVAFFQRPLKDFSHLVGACTFFEEEKRACKNSYSAWEFVALT |
| | KIINEIKSLEKISGEIVPTQTINEVLNLILDKGSITYKKFRSCINLHESI |
| | SFKSLKYDKENAENAKLIDFRKLVEFKKALGVHSLSRQELDQIST |
| | HITLIKDNVKLKTVLEKYNLSNEQINNLLEIEFNDYINLSFKALGM |
| | ILPLMREGKRYDEACEIANLKPKTVDEKKDFLPAFCDSIFAHELSN |
| | PVVNRAISEYRKVLNALLKKYGKVHKIHLELARDVGLSKKAREK |
| | IEKEQKENQAVNAWALKECENIGLKASAKNILKLKLWKEQKEICI |
| | YSGNKISIEHLKDEKALEVDHIYPYSRSFDDSFINKVLVFTKENQE |
| | KLNKTPFEAFGKNIEKWSKIQTLAQNLPYKKKNKILDENFKDKQ |
| | QEDFISRNLNDTRYIATLIAKYTKEYLNFLLLSENENANLKSGEKG |
| | SKIHVQTISGMLTSVLRHTWGFDKKDRNNHLHHALDAIIVAYSTN |
| | SIIKAFSDFRKNQELLKARFYAKELTSDNYKHQVKFFEPFKSFREK |
| | ILSKIDEIFVSKPPRKRARRALHKDTFHSENKIIDKCSYNSKEGLQI |
| | ALSCGRVRKIGTKYVENDTIVRVDIFKKQNKFYAIPIYAMDFALGI |
| | LPNKIVITGKDKNNNPKQWQTIDESYEFCFSLYKNDLILLQKKNM |
| | QEPEFAYYNDFSISTSSICVEKHDNKFENLTSNQKLLFSNAKEGSV |
| | KVESLGIQNLKVFEKYIITPLGDKIKADFQPRENISLKTSKKYGLR* |
| T. denticola Cas9 | MKKEIKDYFLGLDVGTGSVGWAVTDTDYKLLKANRKDLWGMR |
| | CFETAETAEVRRLHRGARRRIERRKKRIKLLQELFSQEIAKTDEGF |
| | FQRMKESPFYAEDKTILQENTLFNDKDFADKTYHKAYPTINHLIK |
| | AWIENKVKPDPRLLYLACHNIIKKRGHFLFEGDFDSENQFDTSIQA |
| | LFEYLREDMEVDIDADSQKVKEILKDSSLKNSEKQSRLNKILGLK |
| | PSDKQKKAITNLISGNKINFADLYDNPDLKDAEKNSISFSKDDFDA |
| | LSDDLASILGDSFELLLKAKAVYNCSVLSKVIGDEQYLSFAKVKI |
| | YEKHKTDLTKLKNVIKKHFPKDYKKVFGYNKNEKNNNYSGYV |
| | GVCKTKSKKLIINNSVNQEDFYKFLKTILSAKSEIKEVNDILTEIET |
| | GTFLPKQISKSNAEIPYQLRKMELEKILSNAEKHFSFLKQKDEKGL |
| | SHSEKIIMLLTFKIPYYIGPINDNHKKFFPDRCWVVKKEKSPSGKT |
| | TPWNFFDHIDKEKTAEAFITSRTNFCTYLVGESVLPKSSLLYSEYT |
| | VLNEINNLQIIIDGKNICDIKLKQKIYEDLFKKYKKITQKQISTFIKH |
| | EGICNKTDEVIILGIDKECTSSLKSYIELKNIFGKQVDEISTKNMLE |
| | EIIRWATIYDEGEGKTILKTKIKAEYGKYCSDEQIKKILNLKFSGW |
| | GRLSRKFLETVTSEMPGFSEPVNIITAMRETQNNLMELLSSEFTFT |
| | ENIKKINSGFEDAEKQFSYDGLVKPLFLSPSVKKMLWQTLKLVKE |
| | ISHITQAPPKKIFIEMAKGAELEPARTKTRLKILQDLYNNCKNDAD |
| | AFSSEIKDLSGKIENEDNLRLRSDKLYLYYTQLGKCMYCGKPIEIG |
| | HVFDTSNYDIDHIYPQSKIKDDSISNRVLVCSSCNKNKEDKYPLKS |
| | EIQSKQRGFWNFLQRNNFISLEKLNRLTRATPISDDETAKFIARQL |
| | VETRQATKVAAKVLEKMFPETKIVYSKAETVSMFRNKFDIVKCR |
| | EINDFHHAHDAYLNIVVGNVYNTKFTNNPWNFIKEKRDNPKIAD |
| | TYNYYKVFDYDVKRNNITAWEKGKTIITVKDMLKRNTPIYTRQA |
| | ACKKGELFNQTIMKKGLGQHPLKKEGPFSNISKYGGYNKVSAAY |
| | YTLIEYEEKGNKIRSLETIPLYLVKDIQKDQDVLKSYLTDLLGKKE |
| | FKILVPKIKINSLLKINGFPCHITGKTNDSFLLRPAVQFCCSNNEVL |
| | YFKKIIRFSEIRSQREKIGKTISPYEDLSFRSYIKENLWKKTKNDEIG |
| | EKEFYDLLQKKNLEIYDMLLTKHKDTIYKKRPNSATIDILVKGKE |
| | KFKSLIIENQFEVILEILKLFSATRNVSDLQHIGGSKYSGVAKIGNK |
| | ISSLDNCILIYQSITGIFEKRIDLLKV* |
| S. mutans Cas9 | MKKPYSIGLDIGTNSVGWAVVTDDYKVPAKKMKVLGNTDKSHI |
| | EKNLLGALLFDSGNTAEDRRLKRTARRRYTRRRNRILYLQEIFSE |
| | EMGKVDDSFFHRLEDSFLVTEDKRGERHPIFGNLEEEVKYHENFP |
| | TIYHLRQYLADNPEKVDLRLVYLALAHIIKFRGHFLIEGKFDTRN |
| | NDVQRLFQEFLAVYDNTFENSSLQEQNVQVEEILTDKISKSAKKD |
| | RVLKLFPNEKSNGRFAEFLKLIVGNQADFKKHFELEEKAPLQFSK |
| | DTYEEELEVLLAQIGDNYAELFLSAKKLYDSILLSGILTVTDVGTK |
| | APLSASMIQRYNEHQMDLAQLKQFIRQKLSDKYNEVFSDVSKDG |
| | YAGYIDGKTNQEAFYKYLKGLLNKIEGSGYFLDKIEREDFLRKQR |
| | TFDNGSIPHQIHLQEMRAIIRRQAEFYPFLADNQDRIEKLLTFRIPY |
| | YVGPLARGKSDFAWLSRKSADKITPWNFDEIVDKESSAEAFINRM |
| | TNYDLYLPNQKVLPKHSLLYEKFTVYNELTKVKYKTEQGKTAFF |
| | DANMKQEIFDGVFKVYRKVTKDKLMDFLEKEFDEFRIVDLTGLD |
| | KENKVFNASYGTYHDLCKILDKDFLDNSKNEKILEDIVLTLTLFE |
| | DREMIRKRLENYSDLLTKEQVKKLERRHYTGWGRLSAELIHGIR |
| | NKESRKTILDYLIDDGNSNRNFMQLINDDALSFKEEIAKAQVIGET |
| | DNLNQVVSDIAGSPAIKKGILQSLKIVDELVKIMGHQPENIVVEM |
| | ARENQFTNQGRRNSQQRLKGLTDSIKEFGSQILKEHPVENSQLQN |
| | DRLFLYYLQNGRDMYTGEELDIDYLSQYDIDHIIPQAFIKDNSIDN |
| | RVLTSSKENRGKSDDVPSKDVVRKMKSYWSKLLSAKLITQRKFD |
| | NLTKAERGGLTDDDKAGFIKRQLVETRQITKHVARILDERFNTET |
| | DENNKKIRQVKIVTLKSNLVSNFRKEFELYKVREINDYHHAHDA |
| | YLNAVIGKALLGVYPQLEPEFVYGDYPHFHGHKENKATAKKFFY |
| | SNIMNFFKKDDVRTDKNGEIIWKKDEHISNIKKVLSYPQVNIVKK |
| | VEEQTGGFSKESILPKGNSDKLIPRKTKKFYWDTKKYGGFDSPIV |
| | AYSILVIADIEKGKSKKLKTVKALVGVTIMEKMTFERDPVAFLER |
| | KGYRNVQEENIIKLPKYSLFKLENGRKRLLASARELQKGNEIVLP |
| | NHLGTLLYHAKNIHKVDEPKHLDYVDKHKDEFKELLDVVSNFSK |
| | KYTLAEGNLEKIKELYAQNNGEDLKELASSFINLLTFTAIGAPATF |
| | KFFDKNIDRKRYTSTTEILNATLIHQSITGLYETRIDLNKLGGD |
| S. thermophilus | MTKPYSIGLDIGTNSVGWAVTTDNYKVPSKKMKVLGNTSKKYIK |
| CRISPR 3 Cas9 | KNLLGVLLFDSGITAEGRRLKRTARRRYTRRRNRILYLQEIFSTEM |
| | ATLDDAFFQRLDDSFLVPDDKRDSKYPIFGNLVEEKAYHDEFPTI |
| | YHLRKYLADSTKKADLRLVYLALAHMIKYRGHFLIEGEFNSKNN |
| | DIQKNFQDFLDTYNAIFESDLSLENSKQLEEIVKDKISKLEKKDRIL |
| | KLFPGEKNSGIFSEFLKLIVGNQADFRKCFNLDEKASLHFSKESYD |
| | EDLETLLGYIGDDYSDVFLKAKKLYDAILLSGFLTVTDNETEAPL |
| | SSAMIKRYNEHKEDLALLKEYIRNISLKTYNEVFKDDTKNGYAG |
| | YIDGKTNQEDFYVYLKKLLAEFEGADYFLEKIDREDFLRKQRTFD |
| | NGSIPYQIHLQEMRAILDKQAKFYPFLAKNKERIEKILTFRIPYYV |
| | GPLARGNSDFAWSIRKRNEKITPWNFEDVIDKESSAEAFINRMTSF |
| | DLYLPEEKVLPKHSLLYETFNVYNELTKVRFIAESMRDYQFLDSK |
| | QKKDIVRLYFKDKRKVTDKDIIEYLHAIYGYDGIELKGIEKQFNSS |
| | LSTYHDLLNIINDKEFLDDSSNEAIIEEIIHTLTIFEDREMIKQRLSKF |
| | ENIFDKSVLKKLSRRHYTGWGKLSAKLINGIRDEKSGNTILDYLID |
| | DGISNRNFMQLIHDDALSFKKKIQKAQIIGDEDKGNIKEVVKSLPG |
| | SPAIKKGILQSIKIVDELVKVMGGRKPESIVVEMARENQYTNQGK |
| | SNSQQRLKRLEKSLKELGSKILKENIPAKLSKIDNNALQNDRLYL |
| | YYLQNGKDMYTGDDLDIDRLSNYDIDHIIPQAFLKDNSIDNKVLV |
| | SSASNRGKSDDVPSLEVVKKRKTFWYQLLKSKLISQRKFDNLTK |
| | AERGGLSPEDKAGFIQRQLVETRQITKHVARLLDEKFNNKKDEN |
| | NRAVRTVKIITLKSTLVSQFRKDFELYKVREINDFHHAHDAYLNA |
| | VVASALLKKYPKLEPEFVYGDYPKYNSFRERKSATEKVYFYSNI |
| | MNIFKKSISLADGRVIERPLIEVNEETGESVWNKESDLATVRRVLS |
| | YPQVNVVKKVEEQNHGLDRGKPKGLFNANLSSKPKPNSNENLV |
| | GAKEYLDPKKYGGYAGISNSFTVLVKGTIEKGAKKKITNVLEFQG |
| | ISILDRINYRKDKLNFLLEKGYKDIELIIELPKYSLFELSDGSRRML |
| | ASILSTNNKRGEIHKGNQIFLSQKFVKLLYHAKRISNTINENHRKY |
| | VENHKKEFEELFYYILEFNENYVGAKKNGKLLNSAFQSWQNHSI |
| | DELCSSFIGPTGSERKGLFELTSRGSAADFEFLGVKIPRYRDYTPSS |
| | LLKDATLIHQSVTGLYETRIDLAKLGEG |
| C. jejuni Cas9 | MARILAFDIGISSIGWAFSENDELKDCGVRIFTKVENPKTGESLAL |
| | PRRLARSARKRLARRKARLNHLKHLIANEFKLNYEDYQSFDESL |
| | AKAYKGSLISPYELRFRALNELLSKQDFARVILHIAKRRGYDDIKN |
| | SDDKEKGAILKAIKQNEEKLANYQSVGEYLYKEYFQKFKENSKE |
| | FTNVRNKKESYERCIAQSFLKDELKLIFKKQREFGFSFSKKFEEEV |
| | LSVAFYKRALKDFSHLVGNCSFFTDEKRAPKNSPLAFMFVALTRII |
| | NLLNNLKNTEGILYTKDDLNALLNEVLKNGTLTYKQTKKLLGLS |
| | DDYEFKGEKGTYFIEFKKYKEFIKALGEHNLSQDDLNEIAKDITLI |
| | KDEIKLKKALAKYDLNQNQIDSLSKLEFKDHLNISFKALKLVTPL |
| | MLEGKKYDEACNELNLKVAINEDKKDFLPAFNETYYKDEVTNPV |
| | VLRAIKEYRKVLNALLKKYGKVHKINIELAREVGKNHSQRAKIE |
| | KEQNENYKAKKDAELECEKLGLKINSKNILKLRLFKEQKEFCAYS |
| | GEKIKISDLQDEKMLEIDHIYPYSRSFDDSYMNKVLVFTKQNQEK |
| | LNQTPFEAFGNDSAKWQKIEVLAKNLPTKKQKRILDKNYKDKEQ |
| | KNFKDRNLNDTRYIARLVLNYTKDYLDFLPLSDDENTKLNDTQK |
| | GSKVHVEAKSGMLTSALRHTWGFSAKDRNNHLHHAIDAVIIAYA |
| | NNSIVKAFSDFKKEQESNSAELYAKKISELDYKNKRKFFEPFSGFR |
| | QKVLDKIDEIFVSKPERKKPSGALHEETFRKEEEFYQSYGGKEGV |
| | LKALELGKIRKVNGKIVKNGDMFRVDIFKHKKTNKFYAVPIYTM |
| | DFALKVLPNKAVARSKKGEIKDWILMDENYEFCFSLYKDSLILIQ |
| | TKDMQEPEFVYYNAFTSSTVSLIVSKHDNKFETLSKNQKILFKNA |
| | NEKEVIAKSIGIQNLKVFEKYIVSALGEVTKAEFRQREDFKK |
| P. multocida Cas9 | MQTTNLSYILGLDLGIASVGWAVVEINENEDPIGLIDVGVRIFERA |
| | EVPKTGESLALSRRLARSTRRLIRRRAHRLLLAKRFLKREGILSTID |
| | LEKGLPNQAWELRVAGLERRLSAIEWGAVLLHLIKHRGYLSKRK |
| | NESQTNNKELGALLSGVAQNHQLLQSDDYRTPAELALKKFAKEE |
| | GHIRNQRGAYTHTFNRLDLLAELNLLFAQQHQFGNPHCKEHIQQ |
| | YMTELLMWQKPALSGEAILKMLGKCTHEKNEFKAAKHTYSAER |
| | FVWLTKLNNLRILEDGAERALNEEERQLLINHPYEKSKLTYAQVR |
| | KLLGLSEQAIFKHLRYSKENAESATFMELKAWHAIRKALENQGL |
| | KDTWQDLAKKPDLLDEIGTAFSLYKTDEDIQQYLTNKVPNSVINA |
| | LLVSLNFDKFIELSLKSLRKILPLMEQGKRYDQACREIYGHHYGE |
| | ANQKTSQLLPAIPAQEIRNPVVLRTLSQARKVINAIIRQYGSPARV |
| | HIETGRELGKSFKERREIQKQQEDNRTKRESAVQKFKELFSDFSSE |
| | PKSKDILKFRLYEQQHGKCLYSGKEINIHRLNEKGYVEIDHALPFS |
| | RTWDDSFNNKVLVLASENQNKGNQTPYEWLQGKINSERWKNFV |
| | ALVLGSQCSAAKKQRLLTQVIDDNKFIDRNLNDTRYIARFLSNYI |
| | QENLLLVGKNKKNVFTPNGQITALLRSRWGLIKARENNNRHHAL |
| | DAIVVACATPSMQQKITRFIRFKEVHPYKIENRYEMVDQESGEIIS |
| | PHFPEPWAYFRQEVNIRVFDNHPDTVLKEMLPDRPQANHQFVQP |
| | LFVSRAPTRKMSGQGHMETIKSAKRLAEGISVLRIPLTQLKPNLLE |
| | NMVNKEREPALYAGLKARLAEFNQDPAKAFATPFYKQGGQQVK |
| | AIRVEQVQKSGVLVRENNGVADNASIVRTDVFIKNNKFFLVPIYT |
| | WQVAKGILPNKAIVAHKNEDEWEEMDEGAKFKFSLFPNDLVELK |
| | TKKEYFFGYYIGLDRATGNISLKEHDGEISKGKDGVYRVGVKLA |
| | LSFEKYQVDELGKNRQICRPQQRQPVR |
| F. novicida Cas9 | MNFKILPIAIDLGVKNTGVFSAFYQKGTSLERLDNKNGKVYELSK |
| | DSYTLLMNNRTARRHQRRGIDRKQLVKRLFKLIWTEQLNLEWD |
| | KDTQQAISFLFNRRGFSFITDGYSPEYLNIVPEQVKAILMDIFDDY |
| | NGEDDLDSYLKLATEQESKISEIYNKLMQKILEFKLMKLCTDIKD |
| | DKVSTKTLKEITSYEFELLADYLANYSESLKTQKFSYTDKQGNLK |
| | ELSYYFIHDKYNIQEFLKRHATINDRILDTLLTDDLDIWNFNFEKF |
| | DFDKNEEKLQNQEDKDHIQAHLHHFVFAVNKIKSEMASGGRHRS |
| | QYFQEITNVLDENNHQEGYLKNFCENLHNKKYSNLSVKNLVNLI |
| | GNLSNLELKPLRKYFNDKIHAKADHWDEQKFTETYCHWILGEW |
| | RVGVKDQDKKDGAKYSYKDLCNELKQKVTKAGLVDFLLELDPC |
| | RTIPPYLDNNNRKPPKCQSLILNPKFLDNQYPNWQQYLQELKKLQ |
| | SIQNYLDSFETDLKVLKSSKDQPYFVEYKSSNQQIASGQRDYKDL |
| | DARILQFIFDRVKASDELLLNEIYFQAKKLKQKASSELEKLESSKK |
| | LDEVIANSQLSQILKSQHTNGIFEQGTFLHLVCKYYKQRQRARDS |
| | RLYIMPEYRYDKKLHKYNNTGRFDDDNQLLTYCNHKPRQKRYQ |
| | LLNDLAGVLQVSPNFLKDKIGSDDDLFISKWLVEHIRGFKKACED |
| | SLKIQKDNRGLLNHKINIARNTKGKCEKEIFNLICKIEGSEDKKGN |
| | YKHGLAYELGVLLFGEPNEASKPEFDRKIKKFNSIYSFAQIQQIAF |
| | AERKGNANTCAVCSADNAHRMQQIKITEPVEDNKDKIILSAKAQ |
| | RLPAIPTRIVDGAVKKMATILAKNIVDDNWQNIKQVLSAKHQLHI |
| | PIITESNAFEFEPALADVKGKSLKDRRKKALERISPENIFKDKNNRI |
| | KEFAKGISAYSGANLTDGDFDGAKEELDHIIPRSHKKYGTLNDEA |
| | NLICVTRGDNKNKGNRIFCLRDLADNYKLKQFETTDDLEIEKKIA |
| | DTIWDANKKDFKFGNYRSFINLTPQEQKAFRHALFLADENPIKQA |
| | VIRAINNRNRTFVNGTQRYFAEVLANNIYLRAKKENLNTDKISFD |
| | YFGIPTIGNGRGIAEIRQLYEKVDSDIQAYAKGDKPQASYSHLIDA |
| | MLAFCIAADEHRNDGSIGLEIDKNYSLYPLDKNTGEVFTKDIFSQI |
| | KITDNEFSDKKLVRKKAIEGFNTHRQMTRDGIYAENYLPILIHKEL |
| | NEVRKGYTWKNSEEIKIFKGKKYDIQQLNNLVYCLKFVDKPISIDI |
| | QISTLEELRNILTTNNIAATAEYYYINLKTQKLHEYYIENYNTALG |
| | YKKYSKEMEFLRSLAYRSERVKIKSIDDVKQVLDKDSNFIIGKITL |
| | PFKKEWQRLYREWQNTTIKDDYEFLKSFFNVKSITKLHKKVRKD |
| | FSLPISTNEGKFLVKRKTWDNNFIYQILNDSDSRADGTKPFIPAFDI |
| | SKNEIVEAIIDSFTSKNIFWLPKNIELQKVDNKNIFAIDTSKWFEVE |
| | TPSDLRDIGIATIQYKIDNNSRPKVRVKLDYVIDDDSKINYFMNHS |
| | LLKSRYPDKVLEILKQSTIIEFESSGFNKTIKEMLGMKLAGIYNETS |
| | NN |
| Lactobacillus | MKVNNYHIGLDIGTSSIGWVAIGKDGKPLRVKGKTAIGARLFQEG |
| buchneri Cas9 | NPAADRRMFRTTRRRLSRRKWRLKLLEEIFDPYITPVDSTFFARL |
| | KQSNLSPKDSRKEFKGSMLFPDLTDMQYHKNYPTIYHLRHALMT |
| | QDKKFDIRMVYLAIHHIVKYRGNFLNSTPVDSFKASKVDFVDQF |
| | KKLNELYAAINPEESFKINLANSEDIGHQFLDPSIRKFDKKKQIPKI |
| | VPVMMNDKVTDRLNGKIASEIIHAILGYKAKLDVVLQCTPVDSK |
| | PWALKFDDEDIDAKLEKILPEMDENQQSIVAILQNLYSQVTLNQI |
| | VPNGMSLSESMIEKYNDHHDHLKLYKKLIDQLADPKKKAVLKK |
| | AYSQYVGDDGKVIEQAEFWSSVKKNLDDSELSKQIMDLIDAEKF |
| | MPKQRTSQNGVIPHQLHQRELDEIIEHQSKYYPWLVEINPNKHDL |
| | HLAKYKIEQLVAFRVPYYVGPMITPKDQAESAETVFSWMERKGT |
| | ETGQITPWNFDEKVDRKASANRFIKRMTTKDTYLIGEDVLPDESL |
| | LYEKFKVLNELNMVRVNGKLLKVADKQAIFQDLFENYKHVSVK |
| | KLQNYIKAKTGLPSDPEISGLSDPEHFNNSLGTYNDFKKLFGSKV |
| | DEPDLQDDFEKIVEWSTVFEDKKILREKLNEITWLSDQQKDVLES |
| | SRYQGWGRLSKKLLTGIVNDQGERIIDKLWNTNKNFMQIQSDDD |
| | FAKRIHEANADQMQAVDVEDVLADAYTSPQNKKAIRQVVKVVD |
| | DIQKAMGGVAPKYISIEFTRSEDRNPRRTISRQRQLENTLKDTAKS |
| | LAKSINPELLSELDNAAKSKKGLTDRLYLYFTQLGKDIYTGEPINI |
| | DELNKYDIDHILPQAFIKDNSLDNRVLVLTAVNNGKSDNVPLRMF |
| | GAKMGHFWKQLAEAGLISKRKLKNLQTDPDTISKYAMHGFIRRQ |
| | LVETSQVIKLVANILGDKYRNDDTKIIEITARMNHQMRDEFGFIK |
| | NREINDYHHAFDAYLTAFLGRYLYHRYIKLRPYFVYGDFKKFRE |
| | DKVTMRNFNFLHDLTDDTQEKIADAETGEVIWDRENSIQQLKDV |
| | YHYKFMLISHEVYTLRGAMFNQTVYPASDAGKRKLIPVKADRPV |
| | NVYGGYSGSADAYMAIVRIHNKKGDKYRVVGVPMRALDRLDA |
| | AKNVSDADFDRALKDVLAPQLTKTKKSRKTGEITQVIEDFEIVLG |
| | KVMYRQLMIDGDKKFMLGSSTYQYNAKQLVLSDQSVKTLASKG |
| | RLDPLQESMDYNNVYTEILDKVNQYFSLYDMNKFRHKLNLGFSK |
| | FISFPNHNVLDGNTKVSSGKREILQEILNGLHANPTFGNLKDVGIT |
| | TPFGQLQQPNGILLSDETKIRYQSPTGLFERTVSLKDL |
| Listeria innocua | MKKPYTIGLDIGTNSVGWAVLTDQYDLVKRKMKIAGDSEKKQIK |
| Cas9 | KNFWGVRLFDEGQTAADRRMARTARRRIERRRNRISYLQGIFAE |
| | EMSKTDANFFCRLSDSFYVDNEKRNSRHPFFATIEEEVEYHKNYP |
| | TIYHLREELVNSSEKADLRLVYLALAHIIKYRGNFLIEGALDTQNT |
| | SVDGIYKQFIQTYNQVFASGIEDGSLKKLEDNKDVAKILVEKVTR |
| | KEKLERILKLYPGEKSAGMFAQFISLIVGSKGNFQKPFDLIEKSDIE |
| | CAKDSYEEDLESLLALIGDEYAELFVAAKNAYSAVVLSSIITVAET |
| | ETNAKLSASMIERFDTHEEDLGELKAFIKLHLPKHYEEIFSNTEKH |
| | GYAGYIDGKTKQADFYKYMKMTLENIEGADYFIAKIEKENFLRK |
| | QRTFDNGAIPHQLHLEELEAILHQQAKYYPFLKENYDKIKSLVTF |
| | RIPYFVGPLANGQSEFAWLTRKADGEIRPWNIEEKVDFGKSAVDF |
| | IEKMTNKDTYLPKENVLPKHSLCYQKYLVYNELTKVRYINDQGK |
| | TSYFSGQEKEQIFNDLFKQKRKVKKKDLELFLRNMSHVESPTIEG |
| | LEDSFNSSYSTYHDLLKVGIKQEILDNPVNTEMLENIVKILTVFED |
| | KRMIKEQLQQFSDVLDGVVLKKLERRHYTGWGRLSAKLLMGIR |
| | DKQSHLTILDYLMNDDGLNRNLMQLINDSNLSFKSIIEKEQVTTA |
| | DKDIQSIVADLAGSPAIKKGILQSLKIVDELVSVMGYPPQTIVVEM |
| | ARENQTTGKGKNNSRPRYKSLEKAIKEFGSQILKEHPTDNQELRN |
| | NRLYLYYLQNGKDMYTGQDLDIHNLSNYDIDHIVPQSFITDNSID |
| | NLVLTSSAGNREKGDDVPPLEIVRKRKVFWEKLYQGNLMSKRKF |
| | DYLTKAERGGLTEADKARFIHRQLVETRQITKNVANILHQRFNYE |
| | KDDHGNTMKQVRIVTLKSALVSQFRKQFQLYKVRDVNDYHHAH |
| | DAYLNGVVANTLLKVYPQLEPEFVYGDYHQFDWFKANKATAK |
| | KQFYTNIMLFFAQKDRIIDENGEILWDKKYLDTVKKVMSYRQMN |
| | IVKKTEIQKGEFSKATIKPKGNSSKLIPRKTNWDPMKYGGLDSPN |
| | MAYAVVIEYAKGKNKLVFEKKIIRVTIMERKAFEKDEKAFLEEQ |
| | GYRQPKVLAKLPKYTLYECEEGRRRMLASANEAQKGNQQVLPN |
| | HLVTLLHHAANCEVSDGKSLDYIESNREMFAELLAHVSEFAKRY |
| | TLAEANLNKINQLFEQNKEGDIKAIAQSFVDLMAFNAMGAPASF |
| | KFFETTIERKRYNNLKELLNSTIIYQSITGLYESRKRLDD |
| L. pneumophiha | MESSQILSPIGIDLGGKFTGVCLSHLEAFAELPNHANTKYSVILIDH |
| Cas9 | NNFQLSQAQRRATRHRVRNKKRNQFVKRVALQLFQHILSRDLNA |
| | KEETALCHYLNNRGYTYVDTDLDEYIKDETTINLLKELLPSESEH |
| | NFIDWFLQKMQSSEFRKILVSKVEEKKDDKELKNAVKNIKNFITG |
| | FEKNSVEGHRHRKVYFENIKSDITKDNQLDSIKKKIPSVCLSNLLG |
| | HLSNLQWKNLHRYLAKNPKQFDEQTFGNEFLRMLKNFRHLKGS |
| | QESLAVRNLIQQLEQSQDYISILEKTPPEITIPPYEARTNTGMEKDQ |
| | SLLLNPEKLNNLYPNWRNLIPGIIDAHPFLEKDLEHTKLRDRKRIIS |
| | PSKQDEKRDSYILQRYLDLNKKIDKFKIKKQLSFLGQGKQLPANLI |
| | ETQKEMETHFNSSLVSVLIQIASAYNKEREDAAQGIWFDNAFSLC |
| | ELSNINPPRKQKILPLLVGAILSEDFINNKDKWAKFKIFWNTHKIG |
| | RTSLKSKCKEIEEARKNSGNAFKIDYEEALNHPEHSNNKALIKIIQ |
| | TIPDIIQAIQSHLGHNDSQALIYHNPFSLSQLYTILETKRDGFHKNC |
| | VAVTCENYWRSQKTEIDPEISYASRLPADSVRPFDGVLARMMQR |
| | LAYEIAMAKWEQIKHIPDNSSLLIPIYLEQNRFEFEESFKKIKGSSS |
| | DKTLEQAIEKQNIQWEEKFQRIINASMNICPYKGASIGGQGEIDHI |
| | YPRSLSKKHFGVIFNSEVNLIYCSSQGNREKKEEHYLLEHLSPLYL |
| | KHQFGTDNVSDIKNFISQNVANIKKYISFHLLTPEQQKAARHALFL |
| | DYDDEAFKTITKFLMSQQKARVNGTQKFLGKQIMEFLSTLADSK |
| | QLQLEFSIKQITAEEVHDHRELLSKQEPKLVKSRQQSFPSHAIDAT |
| | LTMSIGLKEFPQFSQELDNSWFINHLMPDEVHLNPVRSKEKYNKP |
| | NISSTPLFKDSLYAERFIPVWVKGETFAIGFSEKDLFEIKPSNKEKL |
| | FTLLKTYSTKNPGESLQELQAKSKAKWLYFPINKTLALEFLHHYF |
| | HKEIVTPDDTTVCHFINSLRYYTKKESITVKILKEPMPVLSVKFESS |
| | KKNVLGSFKHTIALPATKDWERLFNHPNFLALKANPAPNPKEFNE |
| | FIRKYFLSDNNPNSDIPNNGHNIKPQKHKAVRKVFSLPVIPGNAGT |
| | MMRIRRKDNKGQPLYQLQTIDDTPSMGIQINEDRLVKQEVLMDA |
| | YKTRNLSTIDGINNSEGQAYATFDNWLTLPVSTFKPEIIKLEMKPH |
| | SKTRRYIRITQSLADFIKTIDEALMIKPSDSIDDPLNMPNEIVCKNK |
| | LFGNELKPRDGKMKIVSTGKIVTYEFESDSTPQWIQTLYVTQLKK |
| | QP |
| N. lactamica Cas9 | MAAFKPNPMNYILGLDIGIASVGWAMVEVDEEENPIRLIDLGVRV |
| | FERAEVPKTGDSLAMARRLARSVRRLTRRRAHRLLRARRLLKRE |
| | GVLQDADFDENGLVKSLPNTPWQLRAAALDRKLTCLEWSAVLL |
| | HLVKHRGYLSQRKNEGETADKELGALLKGVADNAHALQTGDFR |
| | TPAELALNKFEKESGHIRNQRGDYSHTFSRKDLQAELNLLFEKQK |
| | EFGNPHVSDGLKEDIETLLMAQRPALSGDAVQKMLGHCTFEPAE |
| | PKAAKNTYTAERFIWLTKLNNLRILEQGSERPLTDTERATLMDEP |
| | YRKSKLTYAQARKLLGLEDTAFFKGLRYGKDNAEASTLMEMKA |
| | YHAISRALEKEGLKDKKSPLNLSTELQDEIGTAFSLFKTDKDITGR |
| | LKDRVQPEILEALLKHISFDKFVQISLKALRRIVPLMEQGKRYDEA |
| | CAEIYGDHYCKKNAEEKIYLPPIPADEIRNPVVLRALSQARKVINC |
| | VVRRYGSPARIHIETAREVGKSFKDRKEIEKRQEENRKDREKAAA |
| | KFREYFPNFVGEPKSKDILKLRLYEQQHGKCLYSGKEINLVRLNE |
| | KGYVEIDHALPFSRTWDDSFNNKVLVLGSENQNKGNQTPYEYFN |
| | GKDNSREWQEFKARVETSRFPRSKKQRILLQKFDEEGFKERNLN |
| | DTRYVNRFLCQFVADHILLTGKGKRRVFASNGQITNLLRGFWGL |
| | RKVRTENDRHHALDAVVVACSTVAMQQKITRFVRYKEMNAFDG |
| | KTIDKETGEVLHQKAHFPQPWEFFAQEVMIRVFGKPDGKPEFEEA |
| | DTPEKLRTLLAEKLSSRPEAVHEYVTPLFVSRAPNRKMSGQGHM |
| | ETVKSAKRLDEGISVLRVPLTQLKLKGLEKMVNREREPKLYDAL |
| | KAQLETHKDDPAKAFAEPFYKYDKAGSRTQQVKAVRIEQVQKT |
| | GVWVRNHNGIADNATMVRVDVFEKGGKYYLVPIYSWQVAKGIL |
| | PDRAVVAFKDEEDWTVMDDSFEFRFVLYANDLIKLTAKKNEFLG |
| | YFVSLNRATGAIDIRTHDTDSTKGKNGIFQSVGVKTALSFQKNQI |
| | DELGKEIRPCRLKKRPPVR |
| N. meningitides | MAAFKPNPINYILGLDIGIASVGWAMVEIDEDENPICLIDLGVRVF |
| Cas9 | ERAEVPKTGDSLAMARRLARSVRRLTRRRAHRLLRARRLLKREG |
| | VLQAADFDENGLIKSLPNTPWQLRAAALDRKLTPLEWSAVLLHLI |
| | KHRGYLSQRKNEGETADKELGALLKGVADNAHALQTGDFRTPA |
| | ELALNKFEKESGHIRNQRGDYSHTFSRKDLQAELILLFEKQKEFG |
| | NPHVSGGLKEGIETLLMTQRPALSGDAVQKMLGHCTFEPAEPKA |
| | AKNTYTAERFIWLTKLNNLRILEQGSERPLTDTERATLMDEPYRK |
| | SKLTYAQARKLLGLEDTAFFKGLRYGKDNAEASTLMEMKAYHA |
| | ISRALEKEGLKDKKSPLNLSPELQDEIGTAFSLFKTDEDITGRLKD |
| | RIQPEILEALLKHISFDKFVQISLKALRRIVPLMEQGKRYDEACAEI |
| | YGDHYGKKNTEEKIYLPPIPADEIRNPVVLRALSQARKVINGVVR |
| | RYGSPARIHIETAREVGKSFKDRKEIEKRQEENRKDREKAAAKFR |
| | EYFPNFVGEPKSKDILKLRLYEQQHGKCLYSGKEINLGRLNEKGY |
| | VEIDHALPFSRTWDDSFNNKVLVLGSENQNKGNQTPYEYFNGKD |
| | NSREWQEFKARVETSRFPRSKKQRILLQKFDEDGFKERNLNDTRY |
| | VNRFLCQFVADRMRLTGKGKKRVFASNGQITNLLRGFWGLRKV |
| | RAENDRHHALDAVVVACSTVAMQQKITRFVRYKEMNAFDGKTI |
| | DKETGEVLHQKTHFPQPWEFFAQEVMIRVFGKPDGKPEFEEADT |
| | PEKLRTLLAEKLSSRPEAVHEYVTPLFVSRAPNRKMSGQGHMET |
| | VKSAKRLDEGVSVLRVPLTQLKLKDLEKMVNREREPKLYEALKA |
| | RLEAHKDDPAKAFAEPFYKYDKAGNRTQQVKAVRVEQVQKTG |
| | VWVRNHNGIADNATMVRVDVFEKGDKYYLVPIYSWQVAKGILP |
| | DRAVVQGKDEEDWQLIDDSFNFKFSLHPNDLVEVITKKARMFGY |
| | FASCHRGTGNINIRIHDLDHKIGKNGILEGIGVKTALSFQKYQIDEL |
| | GKEIRPCRLKKRPPVR |
| B. longum Cas9 | MLSRQLLGASHLARPVSYSYNVQDNDVHCSYGERCFMRGKRYR |
| | IGIDVGLNSVGLAAVEVSDENSPVRLLNAQSVIHDGGVDPQKNKE |
| | AITRKNMSGVARRTRRMRRRKRERLHKLDMLLGKFGYPVIEPES |
| | LDKPFEEWHVRAELATRYIEDDELRRESISIALRHMARHRGWRNP |
| | YRQVDSLISDNPYSKQYGELKEKAKAYNDDATAAEEESTPAQLV |
| | VAMLDAGYAEAPRLRWRTGSKKPDAEGYLPVRLMQEDNANEL |
| | KQIFRVQRVPADEWKPLFRSVFYAVSPKGSAEQRVGQDPLAPEQ |
| | ARALKASLAFQEYRIANVITNLRIKDASAELRKLTVDEKQSIYDQ |
| | LVSPSSEDITWSDLCDFLGFKRSQLKGVGSLTEDGEERISSRPPRLT |
| | SVQRIYESDNKIRKPLVAWWKSASDNEHEAMIRLLSNTVDIDKV |
| | REDVAYASAIEFIDGLDDDALTKLDSVDLPSGRAAYSVETLQKLT |
| | RQMLTTDDDLHEARKTLFNVTDSWRPPADPIGEPLGNPSVDRVL |
| | KNVNRYLMNCQQRWGNPVSVNIEHVRSSFSSVAFARKDKREYE |
| | KNNEKRSIFRSSLSEQLRADEQMEKVRESDLRRLEAIQRQNGQCL |
| | YCGRTITFRTCEMDHIVPRKGVGSTNTRTNFAAVCAECNRMKSN |
| | TPFAIWARSEDAQTRGVSLAEAKKRVTMFTFNPKSYAPREVKAF |
| | KQAVIARLQQTEDDAAIDNRSIESVAWMADELHRRIDWYFNAKQ |
| | YVNSASIDDAEAETMKTTVSVFQGRVTASARRAAGIEGKIHFIGQ |
| | QSKTRLDRRHHAVDASVIAMMNTAAAQTLMERESLRESQRLIGL |
| | MPGERSWKEYPYEGTSRYESFHLWLDNMDVLLELLNDALDNDR |
| | IAVMQSQRYVLGNSIAHDATIHPLEKVPLGSAMSADLIRRASTPA |
| | LWCALTRLPDYDEKEGLPEDSHREIRVHDTRYSADDEMGFFASQ |
| | AAQIAVQEGSADIGSAIHHARVYRCWKTNAKGVRKYFYGMIRVF |
| | QTDLLRACHDDLFTVPLPPQSISMRYGEPRVVQALQSGNAQYLG |
| | SLVVGDEIEMDFSSLDVDGQIGEYLQFFSQFSGGNLAWKHWVVD |
| | GFFNQTQLRIRPRYLAAEGLAKAFSDDVVPDGVQKIVTKQGWLP |
| | PVNTASKTAVRIVRRNAFGEPRLSSAHHMPCSWQWRHE |
| A. muciniphila Cas9 | MSRSLTFSFDIGYASIGWAVIASASHDDADPSVCGCGTVLFPKDD |
| | CQAFKRREYRRLRRNIRSRRVRIERIGRLLVQAQIITPEMKETSGH |
| | PAPFYLASEALKGHRTLAPIELWHVLRWYAHNRGYDNNASWSN |
| | SLSEDGGNGEDTERVKHAQDLMDKHGTATMAETICRELKLEEG |
| | KADAPMEVSTPAYKNLNTAFPRLIVEKEVRRILELSAPLIPGLTAEI |
| | IELIAQHHPLTTEQRGVLLQHGIKLARRYRGSLLFGQLIPRFDNRII |
| | SRCPVTWAQVYEAELKKGNSEQSARERAEKLSKVPTANCPEFYE |
| | YRMARILCNIRADGEPLSAEIRRELMNQARQEGKLTKASLEKAIS |
| | SRLGKETETNVSNYFTLHPDSEEALYLNPAVEVLQRSGIGQILSPS |
| | VYRIAANRLRRGKSVTPNYLLNLLKSRGESGEALEKKIEKESKKK |
| | EADYADTPLKPKYATGRAPYARTVLKKVVEEILDGEDPTRPARG |
| | EAHPDGELKAHDGCLYCLLDTDSSVNQHQKERRLDTMTNNHLV |
| | RHRMLILDRLLKDLIQDFADGQKDRISRVCVEVGKELTTFSAMDS |
| | KKIQRELTLRQKSHTDAVNRLKRKLPGKALSANLIRKCRIAMDM |
| | NWTCPFTGATYGDHELENLELEHIVPHSFRQSNALSSLVLTWPGV |
| | NRMKGQRTGYDFVEQEQENPVPDKPNLHICSLNNYRELVEKLDD |
| | KKGHEDDRRRKKKRKALLMVRGLSHKHQSQNHEAMKEIGMTE |
| | GMMTQSSHLMKLACKSIKTSLPDAHIDMIPGAVTAEVRKAWDVF |
| | GVFKELCPEAADPDSGKILKENLRSLTHLHHALDACVLGLIPYIIP |
| | AHHNGLLRRVLAMRRIPEKLIPQVRPVANQRHYVLNDDGRMML |
| | RDLSASLKENIREQLMEQRVIQHVPADMGGALLKETMQRVLSVD |
| | GSGEDAMVSLSKKKDGKKEKNQVKASKLVGVFPEGPSKLKALK |
| | AAIEIDGNYGVALDPKPVVIRHIKVFKRIMALKEQNGGKPVRILK |
| | KGMLIHLTSSKDPKHAGVWRIESIQDSKGGVKLDLQRAHCAVPK |
| | NKTHECNWREVDLISLLKKYQMKRYPTSYTGTPR |
| O. laneus Cas9 | METTLGIDLGTNSIGLALVDQEEHQILYSGVRIFPEGINKDTIGLGE |
| | KEESRNATRRAKRQMRRQYFRKKLRKAKLLELLIAYDMCPLKPE |
| | DVRRWKNWDKQQKSTVRQFPDTPAFREWLKQNPYELRKQAVT |
| | EDVTRPELGRILYQMIQRRGFLSSRKGKEEGKIFTGKDRMVGIDE |
| | TRKNLQKQTLGAYLYDIAPKNGEKYRFRTERVRARYTLRDMYIR |
| | EFEIIWQRQAGHLGLAHEQATRKKNIFLEGSATNVRNSKLITHLQ |
| | AKYGRGHVLIEDTRITVTFQLPLKEVLGGKIEIEEEQLKFKSNESV |
| | LFWQRPLRSQKSLLSKCVFEGRNFYDPVHQKWIIAGPTPAPLSHP |
| | EFEEFRAYQFINNIIYGKNEHLTAIQREAVFELMCTESKDFNFEKIP |
| | KHLKLFEKFNFDDTTKVPACTTISQLRKLFPHPVWEEKREEIWHC |
| | FYFYDDNTLLFEKLQKDYALQTNDLEKIKKIRLSESYGNVSLKAI |
| | RRINPYLKKGYAYSTAVLLGGIRNSFGKRFEYFKEYEPEIEKAVC |
| | RILKEKNAEGEVIRKIKDYLVHNRFGFAKNDRAFQKLYHHSQAIT |
| | TQAQKERLPETGNLRNPIVQQGLNELRRTVNKLLATCREKYGPSF |
| | KFDHIHVEMGRELRSSKTEREKQSRQIRENEKKNEAAKVKLAEY |
| | GLKAYRDNIQKYLLYKEIEEKGGTVCCPYTGKTLNISHTLGSDNS |
| | VQIEHIIPYSISLDDSLANKTLCDATFNREKGELTPYDFYQKDPSPE |
| | KWGASSWEEIEDRAFRLLPYAKAQRFIRRKPQESNEFISRQLNDT |
| | RYISKKAVEYLSAICSDVKAFPGQLTAELRHLWGLNNILQSAPDIT |
| | FPLPVSATENHREYYVITNEQNEVIRLFPKQGETPRTEKGELLLTG |
| | EVERKVFRCKGMQEFQTDVSDGKYWRRIKLSSSVTWSPLFAPKPI |
| | SADGQIVLKGRIEKGVFVCNQLKQKLKTGLPDGSYWISLPVISQT |
| | FKEGESVNNSKLTSQQVQLFGRVREGIFRCHNYQCPASGADGNF |
| | WCTLDTDTAQPAFTPIKNAPPGVGGGQIILTGDVDDKGIFHADDD |
| | LHYELPASLPKGKYYGIFTVESCDPTLIPIELSAPKTSKGENLIEGNI |
| | WVDEHTGEVRFDPKKNREDQRHHAIDAIVIALSSQSLFQRLSTYN |
| | ARRENKKRGLDSTEHFPSPWPGFAQDVRQSVVPLLVSYKQNPKT |
| | LCKISKTLYKDGKKIHSCGNAVRGQLHKETVYGQRTAPGATEKS |
| | YHIRKDIRELKTSKHIGKVVDITIRQMLLKHLQENYHIDITQEFNIP |
| | SNAFFKEGVYRIFLPNKHGEPVPIKKIRMKEELGNAERLKDNINQ |
| | YVNPRNNHHVMIYQDADGNLKEEIVSFWSVIERQNQGQPIYQLP |
| | REGRNIVSILQINDTFLIGLKEEEPEVYRNDLSTLSKHLYRVQKLS |
| | GMYYTFRHHLASTLNNEREEFRIQSLEAWKRANPVKVQIDEIGRI |
| | TFLNGPLC |

The term “cell” as used herein may refer to either a prokaryotic or eukaryotic cell, optionally obtained from a subject or a commercially available source.

As used herein, the term “CRISPR” refers to Clustered Regularly Interspaced Short Palindromic Repeats (CRISPR). CRISPR may also refer to a technique or system of sequence-specific genetic manipulation relying on the CRISPR pathway. A CRISPR recombinant expression system can be programmed to cleave a target polynucleotide using a CRISPR endonuclease and a guideRNA or a combination of a crRNA and a tracrRNA. A CRISPR system can be used to cause double stranded or single stranded breaks in a target polynucleotide such as DNA or RNA. A CRISPR system can also be used to recruit proteins or label a target polynucleotide. In some aspects, CRISPR-mediated gene editing utilizes the pathways of nonhomologous end-joining (NHEJ) or homologous recombination to perform the edits. These applications of CRISPR technology are known and widely practiced in the art. See, e.g., U.S. Pat. No. 8,697,359 and Hsu et al. (2014) Cell 156(6): 1262-1278.

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure.

The term “encode” as it is applied to nucleic acid sequences refers to a polynucleotide which is said to “encode” a polypeptide, an mRNA, or an effector RNA if, in its native state or when manipulated by methods well known to those skilled in the art, can be transcribed and/or translated to produce the effector RNA, the mRNA, or an mRNA that can for the polypeptide and/or a fragment thereof. The antisense strand is the complement of such a nucleic acid, and the encoding sequence can be deduced therefrom.

As used herein, the term “expression” or “gene expression” refers to the process by which polynucleotides are transcribed into mRNA and/or the process by which the transcribed mRNA is subsequently translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, the term “functional” may be used to modify any molecule, biological, or cellular material to intend that it accomplishes a particular, specified effect.

The term “gRNA” or “guide RNA” as used herein refers to the guide RNA sequences used to target specific genes for correction employing the CRISPR technique. Techniques of designing gRNAs and donor therapeutic polynucleotides for target specificity are well known in the art. For example, Doench, J., et al. Nature biotechnology 2014; 32(12):1262-7, Mohr, S. et al. (2016) FEBS Journal 283: 3232-38, and Graham, D., et al. Genome Biol. 2015; 16: 260, each incorporated herein in their entirety. gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some embodiments, a gRNA is synthetic (Kelley, M. et al. (2016) J of Biotechnology 233 (2016) 74-83, incorporated by reference herein in its entirety). In some embodiments, a gRNA is engineered to have one or more modifications that improve specificity, binding, or other features of the gRNA. In some embodiments, a gRNA is an enhanced gRNA (“esgRNA”) (Chen B, et al. Cell. 2013; 155:1479-1491. doi: 10.1016/j.cell.2013.12.001, incorporated by reference herein in its entirety).

The term “intein” refers to a class of protein that is able to excise itself and join the remaining portion(s) of the protein via protein splicing. A “split intein” comes from two genes. A non-limiting example of a “split-intein” are the C-intein and N-intein sequences originally derived from N. punctiforme.

The term “isolated” as used herein refers to molecules or biologicals or cellular materials being substantially free from other materials.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “ortholog” is used in reference of another gene or protein and intends a homolog of said gene or protein that evolved from the same ancestral source. Orthologs may or may not retain the same function as the gene or protein to which they are orthologous. Non-limiting examples of Cas9 orthologs include S. aureus Cas9 (“spCas9”), S. thermophiles Cas9, L. pneumophilia Cas9, N. lactamica Cas9, N. meningitides Cas9, B. longum Cas9, A. muciniphila Cas9, and O. laneus Cas9.

The term “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. Non-limiting exemplary promoters include CMV, CBA, CAG, Cbh, EF-1a, PGK, UBC, GUSB, UCOE, hAAT, TBG, Desmin, MCK, C5-12, NSE, Synapsin, PDGF, MecP2, CaMKII, mGluR2, NFL, NFH, nβ2, PPE, ENK, EAAT2, GFAP, MBP, and U6 promoters. An “enhancer” is a region of DNA that can be bound by activating proteins to increase the likelihood or frequency of transcription. Non-limiting exemplary enhancers and posttranscriptional regulatory elements include the CMV enhancer and WPRE.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, the term “recombinant expression system” refers to a genetic construct for the expression of certain genetic material formed by recombination.

As used herein, the term “RNA pseudouridylation” refers to an RNA molecule comprising at least one pseudouridine or the process of modifying an RNA molecule to incorporate at least one pseudouridine. Pseudouridine (Ψ) is an abundant posttranscriptional modification in noncoding RNAs. Pseudouridine differs from uridine in at least two important ways: First, the canonical C—N glycosidic bond is changed to a more inert C—C bond. Second, there is an extra hydrogen bond donor at the N1 of the pseudouridine base. These distinctions cause efficient base stacking and water coordination of pseudouridine, thereby increasing the rigidity of the phosohodiester backbone and thermodynamic stability of the Ψ-A base pair compared to U-A base pair. Due to these properties, pseudouridines are often clustered in important regions of rRNAs (ribosomal RNAs), snRNAs (small nuclear RNAs), and tRNAs (transfer RNAs), contributing to RNA function.

As used herein, the term “RNA pseudouridylation modification protein” or “RPMP” refers to a polypeptide capable of modulating RNA pseudouridylation of a target RNA. In some embodiments, the RPMP is a pseudouridine synthase (PUS). In a cell, PUSs recognize a substrate RNA and catalyze the isomerization of uridine to pseudouridine (“RNA-independent pseudouridylation”). In other embodiments, the RPMP is a box H/ACA ribonucleoprotein (RNP) (“RNA-dependent pseudouridylation”). In some embodiments, a box H/ACA RNP comprises a unique RNA (box H/ACA RNA) and four common core proteins (Cbf5/NAP57/Dyskerin, Nhp2/L7Ae, Nop10, and Garl). In some embodiments, a box H/ACA RNP comprises one, two, three, or all four common core proteins (Cbf5/NAP57/Dyskerin, Nhp2/L7Ae, Nop10, and Garl). If present, the RNA component can serve as a guide that base pairs with the substrate RNA and directs the enzyme (Cbf5) to carry out the pseudouridylation reaction at a specific site. Additional mechanisms of RNA pseudouridylation and RPMPs are described in De Zoysa, M. et al. Enzymes. 2017; 41:151-167, incorporated herein by reference in its entirety. In particular embodiments described herein, the RPMP is all or part of H/ACA ribonucleoprotein complex subunit 4 (DKC1), tRNA pseudouridine synthase A (PUS1), tRNA pseudouridylate synthase 3 (PUS3), pseudouridylate synthase 7 (PUS7), pseudouridylate synthase 7 like (PUSL), and a biological equivalent of each thereof.

As used herein, the term “subject” is intended to mean any eukaryotic organism such as a plant or an animal. In some embodiments, the subject may be a mammal; in further embodiments, the subject may be a bovine, equine, feline, murine, porcine, canine, human, or rat.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

As used herein, the term “vector” intends a recombinant vector that retains the ability to infect and transduce non-dividing and/or slowly-dividing cells and integrate into the target cell's genome. The vector may be derived from or based on a wild-type virus. Aspects of this disclosure relate to an adeno-associated virus vector, an adenovirus vector, and a lentivirus vector.

As used herein, the term “XTEN linker” intends a polypeptide comprising six amino acids repeats (Gly, Ala, Pro, Glu, Ser, Thr). In some embodiments, fusion of an XTEN linker to a protein reduces the rate of clearance and degradation of the fusion protein. In some embodiments, the XTEN linker is unstructured.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement. In some embodiments, a biological equivalent retains the

Applicants have provided herein the polypeptide and/or polynucleotide sequences for use in gene and protein transfer and expression techniques described below. It should be understood, although not always explicitly stated that the sequences provided herein can be used to provide the expression product as well as substantially identical sequences that produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

### Modes of Carrying Out the Disclosure

Natural eukaryotic noncoding box H/ACA guide RNAs direct site-specific pseudouridylation by PUS family proteins on spliceosomal small nuclear RNA and ribosomal RNA (rRNA), and assume a functional hairpin-hinge-hairpin-tail conformation, with a conserved box ‘H’ (5′-ANANNA-3′) in the hinge region and a box ‘ACA’ (5-ACA-3) in the tail 3′ end region. Each hairpin contains a single-stranded internal loop termed the pseudouridylation pocket, consisting of two discontinuous tracts of guide sequences (g1 and g1′, and g2 and g2′) that provide pseudouridylation-site specificity through base-pairing interactions with substrate RNA.

Current systems used to directly pseudouridylate RNA rely on recruitment of endogenous pseudouridylation machinery by exogenously expressed guide RNAs, are not proven to be effective in mammalian systems. The present disclosure utilizes the ability of Cas proteins to bind with picomolar affinity to guide RNA scaffolds/direct repeat hairpins and dual guide architecture to increase both target affinity and specificity, and direct RNA pseudouridylation with higher efficiency and specificity, leading to fewer off-target editing events.

Accordingly, described herein are compositions, kits, systems, and methods useful to programmable RNA pseudouridylation at single-nucleotide resolution using RNA-targeting CRISPR/Cas. In some embodiments, the compositions, kits, systems, and methods also comprise engineered single guide RNA (esgRNA) with extensions either upstream or downstream of the Cas interacting scaffold that mimic the entire hairpin-hinge-hairpin-tail conformation and contain guide pocket tracts that specify the pseudouridylation target.

This approach, termed ‘Cas-directed RNA pseudouridylation’, provides a means to reversibly alter genetic information in a temporal manner, unlike traditional CRISPR/Cas9 driven genomic engineering which relies on permanently altering DNA sequence.

Fusion Proteins

In some aspects, provided herein are fusion proteins comprising, consisting of, or consisting essentially of: (i) a guide nucleotide sequence-programmable RNA binding protein; and (ii) a RNA pseudouridylation modification protein (RPMP) or a biological equivalent thereof. In some embodiments, the RPMP is a pseudouridine synthase (PUS). In other embodiments, the RPMP is a box H/ACA ribonucleoprotein (RNP). In some embodiments, a box H/ACA RNP comprises a unique RNA (box H/ACA RNA) and four common core proteins (Cbf5/NAP57/Dyskerin, Nhp2/L7Ae, Nop10, and Garl). In other embodiments, a box H/ACA RNP comprises one, two, three, or all four common core proteins (Cbf5/NAP57/Dyskerin, Nhp2/L7Ae, Nop10, and Garl). In particular embodiments, the RPMP is all or part of H/ACA ribonucleoprotein complex subunit 4 (DKC1), tRNA pseudouridine synthase A (PUS1), tRNA pseudouridylate synthase 3 (PUS3), pseudouridylate synthase 7 (PUS7), pseudouridylate synthase 7 like (PUSL), and a biological equivalent of each thereof.

In some embodiments, the guide nucleotide sequence-programmable RNA binding protein is all or part of a protein selected from: Cas9, modified Cas9, Cas13a, Cas13b, CasRX/Cas13d, and a biological equivalent of each thereof. In some embodiments, the guide nucleotide sequence-programmable RNA binding protein is all or part of a protein selected from: Steptococcus pyogenes Cas9 (spCas9), Staphilococcus aureus Cas9 (saCas9), Francisella novicida Cas9 (FnCas9), Neisseria meningitidis Cas9 (nmCas9), Streptococcus thermophilus CRISPR 1 Cas9 (St1Cas9), Streptococcus thermophilus CRISPR 3 Cas9 (St3Cas9), and Brevibacillus laterosporus Cas9 (BlatCas9). In some embodiments, the guide nucleotide sequence-programmable RNA binding protein is modified to be nuclease inactive.

In some embodiments, the fusion protein further comprises, consists of, or consists essentially of a linker. In some embodiments, the linker is a peptide linker. In some embodiments, the peptide linker comprises one or more repeats of the tri-peptide GGS. In some embodiments, the linker is an XTEN linker. In other embodiments, the linker is a non-peptide linker. In some embodiments, the non-peptide linker comprises polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker. In some embodiments, the components of the fusion protein are fused via intein-mediated fusion.

In some embodiments, the fusion protein comprises, consists of, or consists essentially of the structure NH₂-[RPMP]-[linker]-[guide nucleotide sequence-programmable RNA binding protein]-COOH. In other embodiments, the fusion protein comprises, consists of, or consists essentially of the structure NH₂-[guide nucleotide sequence-programmable RNA binding protein]-[linker]-[RPMP]-COOH.

In some embodiments, the guide nucleotide sequence-programmable RNA binding protein is bound to a guide RNA (gRNA), a crisprRNA (crRNA), and/or a trans-activating crRNA (tracrRNA). In some embodiments, the gRNA is synthetic. In some embodiments, the gRNA is an esgRNA.

In some embodiments, the RPMP protein is encoded by a polynucleotide having a sequence comprising, consisting of, or consisting essentially of all or part of a sequence selected from NM_001142463, NM_001288747, NM_001363, NM_001002019, NM_001002020, NM_025215, NM_031307, NM_001271985, NM_019042, NM_001318164, NM_001318163, NM_001098614, NM_001098615, NM_001271826, NM_031292, a sequence listed in the Additional Sequences section herein, and a biological equivalent of each thereof.

Polynucleotides and Vectors

In some aspects, provided herein are polynucleotides encoding a fusion protein comprising, consisting of, or consisting essentially of: (i) a guide nucleotide sequence-programmable RNA binding protein; and (ii) an RPMP protein. In some embodiments, the polynucleotides further comprise a nucleic acid sequence encoding a linker peptide.

In some embodiments, provided herein are polynucleotides encoding a guide RNA or a crRNA comprising, consisting of, or consisting essentially of a sequence complementary to a target RNA. In some embodiments, the target RNA is an mRNA. In some embodiments, the target RNA comprises a premature stop codon. In some embodiments, the target RNA is susceptible to nonsense mediated decay. In some embodiments, the gRNA or the crRNA comprises, consists of, or consists essentially of a nucleotide sequence complementary to a target RNA with a mismatch at a uridine residue. In some embodiments, the gRNA or the crRNA comprises a nucleotide sequence that mimics a hairpin-hinge-hairpin-tail conformation. In some embodiments, the gRNA contains a guide pocket tract that specifies a pseudouridylation target.

In some embodiments, the gRNA or crRNA comprises a region of complementarity to the target RNA comprising about 15-30 nucleotides, about 15-40 nucleotides, about 15-50 nucleotides, about 15-60 nucleotides, about 15-70 nucleotides, about 15-80 nucleotides, about 15-90 nucleotides, about 15-100 nucleotides, about 50-150 nucleotides, about 50-200 nucleotides, about 100-300 nucleotides, about 100-500 nucleotides, about 100-1000 nucleotides, about 20-40 nucleotides, about 21-100 nucleotides, about 25-100 nucleotides, about 30-100 nucleotides, about 40-200 nucleotides, or about 25-50 nucleotides in length.

In some aspects, provided herein are vectors comprising, consisting of, or consisting essentially of a polynucleotide encoding a fusion protein comprising, consisting of, or consisting essentially of: (i) a guide nucleotide sequence-programmable RNA binding protein; and (ii) an RPMP protein. In some embodiments, the polynucleotides further comprise a nucleic acid sequence encoding a linker peptide.

In some embodiments, the vector is an adenoviral vector, an adeno-associated viral vector, or a lentiviral vector. In some embodiments, the vector further comprises one or more expression control elements operably linked to the polynucleotide. In some embodiments, the vector further comprises one or more selectable markers.

In some embodiments, the vector further comprises, consists of, or consists essentially of a polynucleotide encoding either (i) a gRNA, or (ii) a crRNA and a tracrRNA. In some embodiments, the gRNA or the crRNA comprises a nucleotide sequence complementary to a target RNA.

Cells

In other aspects, provided herein are cells comprising, consisting of, or consisting essentially of one or more vectors comprising, consisting of, or consisting essentially of a polynucleotide encoding a fusion protein comprising, consisting of, or consisting essentially of: (i) a guide nucleotide sequence-programmable RNA binding protein; and (ii) an RPMP protein. In some embodiments, the polynucleotides further comprise a nucleic acid sequence encoding a linker peptide.

In some aspects, provided herein are cells comprising, consisting of, or consisting essentially of a fusion protein comprising, consisting of, or consisting essentially of: (i) a guide nucleotide sequence-programmable RNA binding protein; and (ii) an RPMP protein.

In some embodiments, the cell is a eukaryotic cell. In other embodiments, the cell is a prokaryotic cell. In some embodiments, the cell is a mammalian cell. In some embodiments, the cell is a bovine, murine, feline, equine, porcine, canine, simian, or human cell. In particular embodiments, the cell is a human cell. In some embodiments, the cell is isolated from a subject.

RNA-Targeted CRISPR Systems

In some aspects, provided herein are systems for modulation of RNA methylation, the systems comprising, consisting of, or consisting essentially of: (i) fusion protein comprising, consisting of, or consisting essentially of: (a) a guide nucleotide sequence-programmable RNA binding protein; and (b) an RPMP protein; and either (ii) a gRNA or (iii) a crRNA and a tracrRNA, wherein the gRNA or the crRNA comprises a sequence complementary to a target mRNA. In some embodiments, the complementary sequence is a spacer sequence. In some embodiments, the gRNA is synthetic. In some embodiments, the gRNA is an esgRNA.

In some aspects, provided herein are systems for upregulating or increasing translation of a target mRNA, the systems comprising, consisting of, or consisting essentially of: (i) fusion protein comprising, consisting of, or consisting essentially of: (a) a guide nucleotide sequence-programmable RNA binding protein; and (b) an RPMP protein; and either (ii) a gRNA or (iii) a crRNA and a tracrRNA, wherein the gRNA or the crRNA comprises a sequence complementary to a target mRNA. In some embodiments, the complementary sequence is a spacer sequence. In some embodiments, the gRNA is synthetic. In some embodiments, the gRNA is an esgRNA.

In some aspects, provided herein are systems for downregulating or decreasing translation of a target mRNA, the systems comprising, consisting of, or consisting essentially of: (i) fusion protein comprising, consisting of, or consisting essentially of: (a) a guide nucleotide sequence-programmable RNA binding protein; and (b) an RPMP protein; and either (ii) a gRNA or (iii) a crRNA and a tracrRNA, wherein the gRNA or the crRNA comprises a sequence complementary to a target mRNA. In some embodiments, the complementary sequence is a spacer sequence. In some embodiments, the gRNA is synthetic. In some embodiments, the gRNA is an esgRNA.

In some embodiments, increasing or upregulating translation refers to an increase in the amount of peptide translated from the target mRNA as compared to a control. In some embodiments, the control comprises a level of peptide translated from the target mRNA in the absence of the fusion protein. In some embodiments, the control comprises the level of the peptide translated from the target mRNA prior to addition of the fusion protein. In some embodiments, translation is increased about 1.1 fold, about 1.2 fold, about 1.3 fold, about 1.4 fold, about 1.5 fold, about 1.6 fold, about 1.7 fold, about 1.8 fold, about 1.9 fold, about 2 fold, about 2.5 fold, about 3 fold, about 4 fold, about 5 fold, about 6 fold, about 7 fold, about 8 fold, about 9 fold, about 10 fold, about 20 fold, about 50 fold, about 100 fold, about 1000 fold, or about 10,000 fold relative to the control.

In some embodiments, decreasing or downregulating translation refers to an decrease in the amount of peptide translated from the target mRNA as compared to a control. In some embodiments, the control comprises a level of peptide translated from the target mRNA in the absence of the fusion protein. In some embodiments, the control comprises the level of the peptide translated from the target mRNA prior to addition of the fusion protein. In some embodiments, translation is decreased about 1.1 fold, about 1.2 fold, about 1.3 fold, about 1.4 fold, about 1.5 fold, about 1.6 fold, about 1.7 fold, about 1.8 fold, about 1.9 fold, about 2 fold, about 2.5 fold, about 3 fold, about 4 fold, about 5 fold, about 6 fold, about 7 fold, about 8 fold, about 9 fold, about 10 fold, about 20 fold, about 50 fold, about 100 fold, about 1000 fold, or about 10,000 fold relative to the control.

The amount of peptide translated can be determined by any method known in the art. Non-limiting examples of suitable methods of detection include Western blots, ELISAs, mass spectrometry, immunohistochemistry, immunofluorescence, and use of a reporter gene such as a fluorescence reporter gene.

In some embodiments of the systems described herein, the target mRNA comprises a PAM sequence. In other embodiments, the target mRNA does not comprise a PAM sequence. In some embodiments, the system comprises a PAMmer oligonucleotide. In other embodiments, the system does not comprise a PAMmer oligonucleotide. In some embodiments, aberrant pseudouridylation of the target mRNA is associated with a disease or condition.

In some embodiments of the systems, the target RNA is an mRNA. In some embodiments, the target RNA comprises a premature stop codon. In some embodiments, the target RNA is susceptible to nonsense mediated decay. In some embodiments, the gRNA or the crRNA comprises, consists of, or consists essentially of a nucleotide sequence complementary to a target RNA with a mismatch at a uridine residue. In some embodiments, the gRNA or the crRNA comprises a nucleotide sequence that mimics a hairpin-hinge-hairpin-tail conformation. In some embodiments, the gRNA contains a guide pocket tract that specifies a pseudouridylation target.

In some embodiments, the gRNA or crRNA comprises a region of complementarity to the target RNA comprising about 15-30 nucleotides, about 15-40 nucleotides, about 15-50 nucleotides, about 15-60 nucleotides, about 15-70 nucleotides, about 15-80 nucleotides, about 15-90 nucleotides, about 15-100 nucleotides, about 50-150 nucleotides, about 50-200 nucleotides, about 100-300 nucleotides, about 100-500 nucleotides, about 100-1000 nucleotides, about 20-40 nucleotides, about 21-100 nucleotides, about 25-100 nucleotides, about 30-100 nucleotides, about 40-200 nucleotides, or about 25-50 nucleotides in length.

Methods

In some aspects, provided herein are methods for modulating RNA pseudouridylation of a target RNA, the methods comprising contacting the target mRNA with a fusion protein according to any of the embodiments described herein, wherein the guide nucleotide sequence-programmable RNA binding protein binds a gRNA or a crRNA that hybridizes to a region of the target RNA. In some embodiments, the gRNA is synthetic. In some embodiments, the gRNA is an esgRNA.

In some aspects, provided herein are methods for treating, preventing, and/or blocking nonsense-mediated RNA decay of a target mRNA, the methods comprising contacting a target mRNA with a fusion protein comprising, consisting of, or consisting essentially of: (i) a guide nucleotide sequence-programmable RNA binding protein; and (ii) a RNA pseudouridylation modification protein (RPMP), or an equivalent thereof, wherein the guide nucleotide sequence-programmable RNA binding protein binds a gRNA or a crRNA that hybridizes to a region of the target RNA. In some embodiments, the target mRNA comprises a PAM sequence or complement thereof. In some embodiments, the target mRNA does not comprise a PAM sequence or complement thereof. In some embodiments, the target mRNA is in a cell. In some embodiments, the cell is a eukaryotic cell. In some embodiments, the cell is a prokaryotic cell. In some embodiments, the eukaryotic cell is a mammalian cell, optionally a bovine, murine, feline, equine, porcine, canine, simian, or human cell. In some embodiments, the cell is in a subject. In some embodiments, the gRNA is synthetic. In some embodiments, the gRNA is an esgRNA.

In some aspects, provided herein are methods for treating a disease or condition associated with RNA pseudouridylation of a target RNA in a subject in need thereof, the methods comprising administering a fusion protein, polynucleotide, vector, viral particle, and/or cell as described herein to the subject, thereby treating the disease or condition associated with RNA pseudouridylation. In some embodiments, the disease or condition associated with RNA pseudouridylation is a disease or condition associated with a premature termination codon and/or nonsense-mediated decay, optionally wherein the disease or condition is selected from the group of Hurler syndrome, cystic fibrosis, Duchenne muscular dystrophy, β-thalassemia, cancer, recessive spinal muscular atrophy, and polycystic kidney disease. In some embodiments, the subject is a human. In some embodiments, the methods further comprise administering to the subject: (i) a gRNA complementary to the target RNA, or (ii) a crRNA complementary to the target RNA and a tracrRNA. In some embodiments, the methods further comprise administering a PAMmer to the subject. In some embodiments, the gRNA is synthetic. In some embodiments, the gRNA is an esgRNA.

In some aspects, provided herein are methods for post-transcriptionally increasing or upregulating gene expression, the methods comprising, consisting of, or consisting essentially of contacting a target mRNA with a fusion protein comprising, consisting of, or consisting essentially of: (a) a guide nucleotide sequence-programmable RNA binding protein; and (b) an RPMP protein, wherein the guide nucleotide sequence-programmable RNA binding protein binds a gRNA or a crRNA that hybridizes to a region of the target RNA. In some embodiments, the gRNA is synthetic. In some embodiments, the gRNA is an esgRNA.

In some embodiments, increasing or upregulating gene expression refers to an increase in the amount of peptide translated from the target mRNA as compared to a control. In some embodiments, the control comprises a level of peptide translated from the target mRNA in the absence of the fusion protein. In some embodiments, the control comprises the level of the peptide translated from the target mRNA prior to addition of the fusion protein. In some embodiments, translation is increased about 1.1 fold, about 1.2 fold, about 1.3 fold, about 1.4 fold, about 1.5 fold, about 1.6 fold, about 1.7 fold, about 1.8 fold, about 1.9 fold, about 2 fold, about 2.5 fold, about 3 fold, about 4 fold, about 5 fold, about 6 fold, about 7 fold, about 8 fold, about 9 fold, about 10 fold, about 20 fold, about 50 fold, about 100 fold, about 1000 fold, or about 10,000 fold relative to the control.

In some aspects, provided herein are methods for post-transcriptionally decreasing or downregulating gene expression, the methods comprising, consisting of, or consisting essentially of contacting a target mRNA with a fusion protein comprising, consisting of, or consisting essentially of: (a) a guide nucleotide sequence-programmable RNA binding protein; and (b) an RPMP protein, wherein the guide nucleotide sequence-programmable RNA binding protein binds a gRNA or a crRNA that hybridizes to a region of the target RNA. In some embodiments, the gRNA is synthetic. In some embodiments, the gRNA is an esgRNA.

In some embodiments, decreasing or downregulating gene expression refers to an decrease in the amount of peptide translated from the target mRNA as compared to a control. In some embodiments, the control comprises a level of peptide translated from the target mRNA in the absence of the fusion protein. In some embodiments, the control comprises the level of the peptide translated from the target mRNA prior to addition of the fusion protein. In some embodiments, translation is decreased about 1.1 fold, about 1.2 fold, about 1.3 fold, about 1.4 fold, about 1.5 fold, about 1.6 fold, about 1.7 fold, about 1.8 fold, about 1.9 fold, about 2 fold, about 2.5 fold, about 3 fold, about 4 fold, about 5 fold, about 6 fold, about 7 fold, about 8 fold, about 9 fold, about 10 fold, about 20 fold, about 50 fold, about 100 fold, about 1000 fold, or about 10,000 fold relative to the control. In some embodiments, the gRNA is synthetic. In some embodiments, the gRNA is an esgRNA.

The amount of peptide translated can be determined by any method known in the art. Non-limiting examples of suitable methods of detection include Western blots, ELISAs, mass spectrometry, immunohistochemistry, immunofluorescence, and use of a reporter gene such as a fluorescence reporter gene.

In some embodiments of the methods described herein, the target mRNA comprises a PAM sequence. In other embodiments, the target mRNA does not comprise a PAM sequence. In some embodiments, the method further comprises providing a PAMmer oligonucleotide. In other embodiments, the method does not comprise providing a PAMmer oligonucleotide. In some embodiments, the target mRNA is in a cell. In some embodiments, the cell is a eukaryotic cell. In some embodiments, the cell is a prokaryotic cell. In some embodiments, the cell is a mammalian cell. In some embodiments, the cell is a bovine, murine, feline, equine, porcine, canine, simian, or human cell. In some embodiments, the cell is a plant cell. In some embodiments, the cell is in a subject.

In some aspects, also provided herein are methods for treating a disease or condition in a subject in need thereof, the methods comprising, consisting of, or consisting essentially of administering a fusion protein comprising, consisting of, or consisting essentially of: (a) a guide nucleotide sequence-programmable RNA binding protein; and (b) an RPMP protein, a polynucleotide encoding the fusion protein, a vector comprising the polynucleotide encoding the fusion protein, or viral particle comprising the vector to the subject, thereby decreasing or downregulating translation of a target mRNA in the subject. In some embodiments, aberrant pseudouridylation of the target mRNA is involved in the etiology of a disease or condition in the subject.

In some embodiments of the methods described herein, the subject is a plant or an animal. In some embodiments, the subject is a mammal. In some embodiments, the mammal is a bovine, equine, porcine, canine, feline, simian, murine or human. In some embodiments, the subject is a human.

In some embodiments of the methods described herein, the subject is further administered (i) a gRNA complementary to the target mRNA, or (ii) a crRNA complementary to the target mRNA and a tracrRNA. In some embodiments, the complementary sequence is a spacer sequence. In some embodiments, the gRNA is synthetic. In some embodiments, the gRNA is an esgRNA.

Viral Particles

In some aspects, provided herein are viral particles comprising, consisting of, or consisting essentially of a vector comprising, consisting of, or consisting essentially of a polynucleotide encoding a fusion protein comprising, consisting of, or consisting essentially of: (i) a guide nucleotide sequence-programmable RNA binding protein; and (ii) an RPMP protein. In some embodiments, the polynucleotides further comprise a nucleic acid sequence encoding a linker peptide.

In general methods of packaging genetic material such as RNA or DNA into one or more vectors is well known in the art. For example, the genetic material may be packaged using a packaging vector and cell lines and introduced via traditional recombinant methods.

In some embodiments, the packaging vector may include, but is not limited to retroviral vector, lentiviral vector, adenoviral vector, and adeno-associated viral vector. The packaging vector contains elements and sequences that facilitate the delivery of genetic materials into cells. For example, the retroviral constructs are packaging plasmids comprising at least one retroviral helper DNA sequence derived from a replication-incompetent retroviral genome encoding in trans all virion proteins required to package a replication incompetent retroviral vector, and for producing virion proteins capable of packaging the replication-incompetent retroviral vector at high titer, without the production of replication-competent helper virus. The retroviral DNA sequence lacks the region encoding the native enhancer and/or promoter of the viral 5′ LTR of the virus, and lacks both the psi function sequence responsible for packaging helper genome and the 3′ LTR, but encodes a foreign polyadenylation site, for example the SV40 polyadenylation site, and a foreign enhancer and/or promoter which directs efficient transcription in a cell type where virus production is desired. The retrovirus is a leukemia virus such as a Moloney Murine Leukemia Virus (MMLV), the Human Immunodeficiency Virus (HIV), or the Gibbon Ape Leukemia virus (GALV). The foreign enhancer and promoter may be the human cytomegalovirus (HCMV) immediate early (IE) enhancer and promoter, the enhancer and promoter (U3 region) of the Moloney Murine Sarcoma Virus (MMSV), the U3 region of Rous Sarcoma Virus (RSV), the U3 region of Spleen Focus Forming Virus (SFFV), or the HCMV IE enhancer joined to the native Moloney Murine Leukemia Virus (MMLV) promoter.

The retroviral packaging plasmid may consist of two retroviral helper DNA sequences encoded by plasmid based expression vectors, for example where a first helper sequence contains a cDNA encoding the gag and pol proteins of ecotropic MMLV or GALV and a second helper sequence contains a cDNA encoding the env protein. The Env gene, which determines the host range, may be derived from the genes encoding xenotropic, amphotropic, ecotropic, polytropic (mink focus forming) or 10A1 murine leukemia virus env proteins, or the Gibbon Ape Leukemia Virus (GALV env protein, the Human Immunodeficiency Virus env (gp160) protein, the Vesicular Stomatitus Virus (VSV) G protein, the Human T cell leukemia (HTLV) type I and II env gene products, chimeric envelope gene derived from combinations of one or more of the aforementioned env genes or chimeric envelope genes encoding the cytoplasmic and transmembrane of the aforementioned env gene products and a monoclonal antibody directed against a specific surface molecule on a desired target cell. Similar vector based systems may employ other vectors such as sleeping beauty vectors or transposon elements.

The resulting packaged expression systems may then be introduced via an appropriate route of administration, discussed in detail with respect to the method aspects disclosed herein.

Compositions

Also provided by this invention is a composition comprising any one or more of the fusion proteins and a carrier. In some embodiments, the carrier is a pharmaceutically acceptable carrier. In some embodiments, the composition is a pharmaceutical composition comprising one or more fusion proteins and a pharmaceutically acceptable carrier. In some embodiments, the composition or pharmaceutical composition further comprises one or more gRNAs, crRNAs, and/or tracrRNAs.

Briefly, pharmaceutical compositions of the present invention may comprise an fusion proteins or a polynucleotide encoding said fusion protein, optionally comprised in an AAV, which is optionally also immune orthogonal, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for oral, intravenous, topical, enteral, and/or parenteral administration. In certain embodiments, the compositions of the present disclosure are formulated for intravenous administration.

Kits

[In some aspects, provided herein are kits comprising, consisting of, or consisting essentially of one or more fusion proteins, polynucleotides encoding a fusion protein, vectors comprising the polynucleotide, or viral particles comprising the vector, wherein the fusion protein comprises, consists of, or consists essentially of: (a) a guide nucleotide sequence-programmable RNA binding protein; and (b) an RPMP protein. In some embodiments, the kits further comprise, consist of, or consist essentially of instructions for use.

[In some embodiments of the kits described herein, the kits further comprise, consist of, or consist essentially of one or more nucleic acids selected from: (i) a gRNA; (ii) a crRNA and a tracrRNA; (iii) a PAMmer oligonucleotide; and (iv) a vector for expressing the nucleic acid of (i), (ii), or (iii). In some embodiments, the gRNA is synthetic. In some embodiments, the gRNA is an esgRNA.

In some embodiments, the kits further comprise, consist of, or consist essentially of one or more reagents for carrying out a method of the disclosure. Non-limiting examples of such reagents comprise viral packaging cells, viral vectors, vector backbones, gRNAs, transfection reagents, transduction reagents, viral particles, and PCR primers.

### Example

A Cas-directed pseudouridylation system was designed that (1) recognizes and edits a reporter mRNA construct in libing cells at a base-specific level, and (2) effectively reverses premature termination codon (PTC) mediated silencing of expression from reporter transcripts in cell culture.

The minimal Cas-directed pseudouridylation system of this example is composed of a nuclease-dead Cas (e.g. dCas9, dCas13) protein fused to the catalytic domain of the human DKC1 protein modules, a single guide RNA (sgRNA) driven by a U6 polymerase III promoter, and an optional inclusion of an antisense synthetic oligonucleotide composed alternating 2′OMe RNA and DNA bases (PAMmer). These are delivered to the nuclei of mammalian cells with transfection reagents that form a complex to bind and edit mRNA after forming an RCas9-RNA recognition complex. This allows for selective RNA modification in which targeted uridine residues are isomerized to pseudouridine to be differentially recognized by the cellular machinery.

The catalytically active pseudourydilation domain consists of wildtype human DKC1, PUS1 or PUS7. These domains are fused to a semi-flexible XTEN peptide linker at its C or N-terminus, which is then fused to dCas9 at its C or N-terminus. To control for RNA-recognition independent background editing, fusion constructs lacking the dCas moiety have also been generated (PX).

The sgRNA construct has been modified with a region of homology capable of near-perfect RNA-RNA base pairing over desired site of editing. The homology region contains a mismatch at the targeted uridine, forcing an mispairing and the generation of a ‘pseudo-dsRNA’ substrate on the target transcript. This generates a means of programmable RNA substrate recognition as well as simultaneous base-specific pseudouridylation. Furthermore, these modified sgRNA constructs have been cloned into a vector also containing an mCherry construct driven by a separate Ef1a pol II promoter. This allows sorting of cells transfected with the sgRNA using flow-cytometry and/or enrichment of cells with targeted RNA modification.

### EQUIVALENTS

It should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

1. Xiao, M., et al., Functionality and substrate specificity of human box H/ACA guide RNAs. RNA, 2009. 15(1): p. 176-86.
2. Karijolich, J., C. Yi, and Y. T. Yu, Transcriptome-wide dynamics of RNA pseudouridylation. Nat Rev Mol Cell Biol, 2015. 16(10): p. 581-5.
3. Huang, C., G. Wu, and Y. T. Yu, Inducing nonsense suppression by targeted pseudouridylation. Nat Protoc, 2012. 7(4): p. 789-800.
4. Karijolich, J. and Y. T. Yu, Converting nonsense codons into sense codons by targeted pseudouridylation. Nature, 2011. 474(7351): p. 395-8.

ADDITIONAL SEQUENCES
DKC1

| FEATURES | Location/Qualifiers |
|---|---|
| source | 1..2593 |
| | /organism = ″Homo sapiens″ |
| | /mol_type = ″mRNA″ |
| | /db_xref = ″taxon:9606″ |
| | /chromosome = ″X″ |
| | /map = ″Xq28″ |
| gene | 1..2593 |
| | /gene = ″DKC1″ |
| | /gene_synonym = ″CBF5; DKC; DKCX; NAP57; NOLA4; XAP101″ |
| | /note = ″dyskerin pseudouridine synthase 1″ |
| | /db_xref = ″GeneID:1736″ |
| | /db_xref = ″HGNC:HGNC:2890″ |
| | /db_xref = ″MIM:300126″ |
| exon | 1..240 |
| | /gene = ″DKC1″ |
| | /gene_synonym = ″CBF5; DKC; DKCX; NAP57; NOLA4; XAP101″ |
| | /inference = ″alignment:Splign:2.1.0″ |
| CDS | 225..1754 |
| | /gene = ″DKC1″ |
| | /gene_synonym = ″CBF5; DKC; DKCX; NAP57; NOLA4; XAP101″ |
| | /note = ″isoform 2 is encoded by transcript variant 2; |
| | H/ACA |
| | ribonucleoprotein complex subunit 4; nucleolar protein |
| | family A member 4; snoRNP protein DKC1; nopp140- |
| | associated |
| | protein of 57 kDa; CBF5 homolog; dyskeratosis |
| | congenita 1, |
| | dyskerin; nucleolar protein NAP57; H/ACA |
| | ribonucleoprotein |
| | complex subunit DKC1″ |
| | /codon_start = 1 |
| | /product = ″H/ACA ribonucleoprotein complex subunit DKC1 |
| | isoform 2″ |
| | /protein_id = ″NP_001135935.1″ |
| | /db_xref = ″GeneID:1736″ |
| | /db_xref = ″HGNC:HGNC:2890″ |
| | /db_xref = ″MIM:300126″ |

/translation = ″MADAEVIILPKKHKKKKERKSLPEEDVAEIQHAEEFLIKPESKV
AKLDTSQWPLLLKNFDKLNVRTTHYTPLACGSNPLKREIGDYIRTGFINLDKPSNPSS
HEVVAWIRRILRVEKTGHSGTLDPKVTGCLIVCIERATRLVKSQQSAGKEYVGIVRLH
NAIEGGTQLSRALETLTGALFQRPPLIAAVKRQLRVRTIYESKMIEYDPERRLGIFWV
SCEAGTYIRTLCVHLGLLLGVGGQMQELRRVRSGVMSEKDHMVTMHDVLDAQWLYDNH
KDESYLRRVVYPLEKLLTSHKRLVMKDSAVNAICYGAKIMLPGVLRYEDGIEVNQEIV
VITTKGEAICMAIALMTTAVISTCDHGIVAKIKRVIMERDTYPRKWGLGPKASQKKLM
IKQGLLDKHGKPTDSTPATWKQDESAKKEVVAEVVKAPQVVAEAAKTAKRKRESESES
DETPPAAPQLIKKEKKKSKKDKKAKAGLESGAEPGDGDSDTTKKKKKKKKAKEVELVS
E″

| | |
|---|---|
| misc_feature | 228..287 |
| | /gene = ″DKC1″ |
| | /gene_synonym = ″CBF5; DKC; DKCX; NAP57; NOLA4; XAP101″ |
| | /experiment = ″experimental evidence, no additional |
| | details |
| | recorded″ |
| | /note = ″propagated from UniProtKB/Swiss-Prot |
| | (O60832.3); |
| | Region: Nucleolar localization″ |
| misc_feature | 228..230 |
| | /gene = ″DKC1″ |
| | /gene_synonym = ″CBF5; DKC; DKCX; NAP57; NOLA4; XAP101″ |
| | /experiment = ″experimental evidence, no additional |
| | details |
| | recorded″ |
| | /note = ″N-acetylalanine. {ECO:0000244\|PubMed:19413330, |
| | ECO:0000244\|PubMed:22223895, ECO:0000269\|Ref.8}; |
| | propagated from UniProtKB/Swiss-Prot(O60832.3); |
| | acetylation site″ |
| misc_feature | 285..287 |
| | /gene = ″DKC1″ |
| | /gene_synonym = ″CBF5; DKC; DKCX; NAP57; NOLA4; XAP101″ |
| | /experiment = ″experimental evidence, no additional |
| | details |
| | recorded″ |
| | /note = ″Phosphoserine. {ECO:0000244\|PubMed:17081983, |
| | ECO:0000244\|PubMed:18669648, |
| | ECO:0000244\|PubMed:18691976, |
| | ECO:0000244\|PubMed:19690332, |
| | ECO:0000244\|PubMed:20068231, |
| | ECO:0000244\|PubMed:21406692}; propagated from |
| | UniProtKB/Swiss-Prot (O60832.3); phosphorylation site″ |
| misc_feature | 1383..1385 |
| | /gene = ″DKC1″ |
| | /gene_synonym = ″CBF5; DKC; DKCX; NAP57; NOLA4; XAP101″ |
| | /experiment = ″experimental evidence, no additional |
| | details |
| | recorded″ |
| | /note = ″Phosphoserine. {ECO:0000244\|PubMed:23186163}; |
| | propagated from UniProtKB/Swiss-Prot (O60832.3); |
| | phosphorylation site″ |
| misc_feature | 1545..1751 |
| | /gene = ″DKC1″ |
| | /gene_synonym = ″CBF5; DKC; DKCX; NAP57; NOLA4; XAP101″ |
| | /experiment = ″experimental evidence, no additional |
| | details |
| | recorded″ |
| | /note = ″propagated from UniProtKB/Swiss-Prot |
| | (O60832.3); |
| | Region: Nuclear and nucleolar localization″ |
| misc_feature | 1560..1562 |
| | /gene = ″DKC1″ |
| | /gene_synonym = ″CBF5; DKC; DKCX; NAP57; NOLA4; XAP101″ |
| | /experiment = ″experimental evidence, no additional |
| | details |
| | recorded″ |
| | /note = ″Phosphoserine. {ECO:0000244\|PubMed:20068231, |
| | ECO:0000244\|PubMed:21406692, |
| | ECO:0000244\|PubMed:23186163}; |
| | propagated from UniProtKB/Swiss-Prot (O60832.3); |
| | phosphorylation site″ |
| misc_feature | 1566..1568 |
| | /gene = ″DKC1″ |
| | /gene_synonym = ″CBF5; DKC; DKCX; NAP57; NOLA4; XAP101″ |
| | /experiment = ″experimental evidence, no additional |
| | details |
| | recorded″ |
| | /note = ″Phosphoserine. {ECO:0000244\|PubMed:20068231, |
| | ECO:0000244\|PubMed:21406692, |
| | ECO:0000244\|PubMed:23186163}; |
| | propagated from UniProtKB/Swiss-Prot (O60832.3); |
| | phosphorylation site″ |
| misc_feature | 1572..1574 |
| | /gene = ″DKC1″ |
| | /gene_synonym = ″CBF5; DKC; DKCX; NAP57; NOLA4; XAP101″ |
| | /experiment = ″experimental evidence, no additional |
| | details |
| | recorded″ |
| | /note = ″Phosphoserine. {ECO:0000244\|PubMed:21406692}; |
| | propagated from UniProtKB/Swiss-Prot (O60832.3); |
| | phosphorylation site″ |
| misc_feature | 1581..1583 |
| | /gene = ″DKC1″ |
| | /gene_synonym = ″CBF5; DKC; DKCX; NAP57; NOLA4; XAP101″ |
| | /experiment = ″experimental evidence, no additional |
| | details |
| | recorded″ |
| | /note = ″Phosphothreonine. |
| | {ECO:0000250\|UniProtKB:Q9ESX5}; |
| | propagated from UniProtKB/Swiss-Prot (O60832.3); |
| | phosphorylation site″ |
| misc_feature | 1662..1664 |
| | /gene = ″DKC1″ |
| | /gene_synonym = ″CBF5; DKC; DKCX; NAP57; NOLA4; XAP101″ |
| | /experiment = ″experimental evidence, no additional |
| | details |
| | recorded″ |
| | /note = ″Phosphoserine. {ECO:0000244\|PubMed:18669648, |
| | ECO:0000244\|PubMed:19690332, |
| | ECO:0000244\|PubMed:20068231, |
| | ECO:0000244\|PubMed:21406692, |
| | ECO:0000244\|PubMed:23186163}; |
| | propagated from UniProtKB/Swiss-Prot (O60832.3); |
| | phosphorylation site″ |
| misc_feature | 1689..1691 |
| | /gene = ″DKC1″ |
| | /gene_synonym = ″CBF5; DKC; DKCX; NAP57; NOLA4; XAP101″ |
| | /experiment = ″experimental evidence, no additional |
| | details |
| | recorded″ |
| | /note = ″Phosphoserine. (ECO:0000244\|PubMed:16964243, |
| | ECO:0000244\|PubMed:18669648, |
| | ECO:0000244\|PubMed:19690332, |
| | ECO:0000244\|PubMed:20068231, |
| | ECO:0000244\|PubMed:21406692, |
| | ECO:0000244\|PubMed:23186163, |
| | ECO:0000244\|PubMed:24275569}; |
| | propagated from UniProtKB/Swiss-Prot (O60832.3); |
| | phosphorylation site″ |
| misc_feature | 1746..1748 |
| | /gene = ″DKC1″ |
| | /gene_synonym = ″CBF5; DKC; DKCX; NAP57; NOLA4; XAP101″ |
| | /experiment = ″experimental evidence, no additional |
| | details |
| | recorded″ |
| | /note = ″Phosphoserine. {ECO:0000244\|PubMed:17081983, |
| | ECO:0000244\|PubMed:19369195, |
| | ECO:0000244\|PubMed:20068231, |
| | ECO:0000244\|PubMed:21406692, |
| | ECO:0000244\|PubMed:23186163}; |
| | propagated from UniProtKB/Swiss-Prot (O60832.3); |
| | phosphorylation site″ |
| exon | 241..308 |
| | /gene = ″DKC1″ |
| | /gene_synonym = ″CBF5; DKC; DKCX; NAP57; NOLA4; XAP101″ |
| | /inference = ″alignment:Splign:2.1.0″ |
| STS | 290..662 |
| | /gene = ″DKC1″ |
| | /gene_synonym = ″CBF5; DKC; DKCX; NAP57; NOLA4; XAP101″ |
| | /standard_name = ″stSG604276″ |
| | /db_xref = ″UniSTS:447593″ |
| exon | 309..395 |
| | /gene = ″DKC1″ |
| | /gene_synonym = ″CBF5; DKC; DKCX; NAP57; NOLA4; XAP101″ |
| | /inference = ″alignment:Splign:2.1.0″ |
| exon | 396..487 |
| | /gene = ″DKC1″ |
| | /gene_synonym = ″CBF5; DKC; DKCX; NAP57; NOLA4; XAP101″ |
| | /inference = ″alignment:Splign:2.1.0″ |
| exon | 488..672 |
| | /gene = ″DKC1″ |
| | /gene_synonym = ″CBF5; DKC; DKCX; NAP57; NOLA4; XAP101″ |
| | /inference = ″alignment:Splign:2.1.0″ |
| exon | 673..737 |
| | /gene = ″DKC1″ |
| | /gene_synonym = ″CBF5; DKC; DKCX; NAP57; NOLA4; XAP101″ |
| | /inference = ″alignment:Splign:2.1.0″ |
| exon | 738..864 |
| | /gene = ″DKC1″ |
| | /gene_synonym = ″CBF5; DKC; DKCX; NAP57; NOLA4; XAP101″ |
| | /inference = ″alignment:Splign:2.1.0″ |
| exon | 865..995 |
| | /gene = ″DKC1″ |
| | /gene_synonym = ″CBF5; DKC; DKCX; NAP57; NOLA4; XAP101″ |
| | /inference = ″alignment:Splign:2.1.0″ |
| exon | 996..1139 |
| | /gene = ″DKC1″ |
| | /gene_synonym = ″CBF5; DKC; DKCX; NAP57; NOLA4; XAP101″ |
| | /inference = ″alignment:Splign:2.1.0″ |
| exon | 1140..1260 |
| | /gene = ″DKC1″ |
| | /gene_synonym = ″CBF5; DKC; DKCX; NAP57; NOLA4; XAP101″ |
| | /inference = ″alignment:Splign:2.1.0″ |
| exon | 1261..1379 |
| | /gene = ″DKC1″ |
| | /gene_synonym = ″CBF5; DKC; DKCX; NAP57; NOLA4; XAP101″ |
| | /inference = ″alignment:Splign:2.1.0″ |
| exon | 1380..1468 |
| | /gene = ″DKC1″ |
| | /gene_synonym = ″CBF5; DKC; DKCX; NAP57; NOLA4; XAP101″ |
| | /inference = ″alignment:Splign:2.1.0″ |
| exon | 1469..1547 |
| | /gene = ″DKC1″ |
| | /gene_synonym = ″CBF5; DKC; DKCX; NAP57; NOLA4; XAP101″ |
| | /inference = ″alignment:Splign:2.1.0″ |
| exon | 1548..1685 |
| | /gene = ″DKC1″ |
| | /gene_synonym = ″CBF5; DKC; DKCX; NAP57; NOLA4; XAP101″ |
| | /inference = ″alignment:Splign:2.1.0″ |
| STS | 1685..1941 |
| | /gene = ″DKC1″ |
| | /gene_synonym = ″CBF5; DKC; DKCX; NAP57; NOLA4; XAP101″ |
| | /standard_name = ″REN90635″ |
| | /db_xref = ″UniSTS:415433″ |
| exon | 1686..2576 |
| | /gene = ″DKC1″ |
| | /gene_synonym = ″CBF5; DKC; DKCX; NAP57; NOLA4; XAP101″ |
| | /inference = ″alignment:Splign:2.1.0″ |
| STS | 1761..2288 |
| | /gene = ″DKC1″ |
| | /gene_synonym = ″CBF5; DKC; DKCX; NAP57; NOLA4; XAP101″ |
| | /standard_name = ″ECD13062″ |
| | /db_xref = ″UniSTS:294093″ |
| STS | 1939..2165 |
| | /gene = ″DKC1″ |
| | /gene_synonym = ″CBF5; DKC; DKCX; NAP57; NOLA4; XAP101″ |
| | /standard_name = ″REN90636″ |
| | /db_xref = ″UniSTS:415434″ |
| STS | 2138..2390 |
| | /gene = ″DKC1″ |
| | /gene_synonym = ″CBF5; DKC; DKCX; NAP57; NOLA4; XAP101″ |
| | /standard_name = ″REN90637″ |
| | /db_xref = ″UniSTS:415435″ |
| STS | 2268..2555 |
| | /gene = ″DKC1″ |
| | /gene_synonym = ″CBF5; DKC; DKCX; NAP57; NOLA4; XAP101″ |
| | /standard_name = ″A004F19″ |
| | /db_xref = ″UniSTS:4842″ |
| STS | 2326..2498 |
| | /gene = ″DKC1″ |
| | /gene_synonym = ″CBF5; DKC; DKCX; NAP57; NOLA4; XAP101″ |
| | /standard_name = ″IB1223″ |
| | /db_xref = ″UniSTS:64040″ |
| regulatory | 2536..2541 |
| | /regulatory_class = ″polyA_signal_sequence″ |
| | /gene = ″DKC1″ |
| | /gene_synonym = ″CBF5; DKC; DKCX; NAP57; NOLA4; XAP101″ |
| polyA_site | 2576 |
| | /gene = ″DKC1″ |
| | /gene_synonym = ″CBF5; DKC; DKCX; NAP57; NOLA4; XAP101″ |

ORIGIN

| | |
|---|---|
| 1 | gtactggccg agccagcaaa tcgcattgcg cagacgacca gcgggcgcct cggattccgc |
| 61 | ccccgggatg gccccgcctc ctcccgcccc gcggcaaggc acgcacaggg cagtgcgcgg |
| 121 | gtgggtgggt cctagcagcg cggcctgacg ggaccaaggc ggcgggagtc tgcggtcgtt |
| 181 | ccctcggctg tggaccgggc ggcacgcacg cggtgcaggg taacatggcg gatgcggaag |
| 241 | taattatttt gccaaagaaa cataagaaga aaaaggagcg gaagtcattg ccagaagaag |
| 301 | atgtagccga aatacaacac gctgaagaat ttcttatcaa acctgaatcc aaagttgcta |
| 361 | agttggacac gtctcagtgg ccccttttgc taaagaattt tgataagctg aatgtaagga |
| 421 | caacacacta tacacctctt gcatgtggtt caaatcctct gaagagagag attggggact |
| 481 | atatcaggac aggtttcatt aatcttgaca agccctctaa cccctcttcc catgaggtgg |
| 541 | tagcctggat tcgacggata cttcgggtgg agaagacagg gcacagtggt actctggatc |
| 601 | ccaaggtgac tggttgttta atcgtgtgca tagaacgagc cactcgcttg gtgaagtcac |
| 661 | aacagagtgc aggcaaagag tatgtgggga ttgtccggct gcacaatgct attgaagggg |
| 721 | ggacccagct ttctagggcc ctagaaactc tgacaggtgc cttattccag cgacccccac |
| 781 | ttattgctgc agtaaagagg cagctccgag tgaggaccat ctacgagagc aaaatgattg |
| 841 | aatacgatcc tgaaagaaga ttaggaatct tttgggtgag ttgtgaggct ggcacctaca |
| 901 | ttcggacatt atgtgtgcac cttggtttgt tattgggagt tggtggtcag atgcaggagc |
| 961 | ttcggagggt tcgttctgga gtcatgagtg aaaaggacca catggtgaca atgcatgatg |
| 1021 | tgcttgatgc tcagtggctg tatgataacc acaaggatga gagttacctg cggcgagttg |
| 1081 | tttacccttt ggaaaagctg ttgacatctc ataaacggct ggttatgaaa gacagtgcag |
| 1141 | taaatgccat ctgctatggg gccaagatta tgcttccagg tgttcttcga tatgaggacg |
| 1201 | gcattgaggt caatcaggag attgtggtta tcaccaccaa aggagaagca atctgcatgg |
| 1261 | ctattgcatt aatgaccaca gcggtcatct ctacctgcga ccatggtata gtagccaaga |
| 1321 | tcaagagagt gatcatggag agagacactt accctcggaa gtggggttta ggtccaaagg |
| 1381 | caagtcagaa gaagctgatg atcaagcagg gccttctgga caagcatggg aagcccacag |
| 1441 | acagcacacc tgccacctgg aagcaggatg agtctgccaa aaaagaggtg gttgctgaag |
| 1501 | tggtaaaagc cccgcaggta gttgccgaag cagcaaaaac tgcgaagcgg aagcgagaga |
| 1561 | gtgagagtga aagtgacgag actcctccag cagctcctca gttgatcaag aaggaaaaga |
| 1621 | agaagagtaa gaaggacaag aaggccaaag ctggtctgga gagcggggcc gagcctggag |
| 1681 | atggggacag tgataccacc aagaagaaga agaagaagaa gaaagcaaaa gaggtagaat |
| 1741 | tggtttctga gtagtgaagg ccacttgaag ctggaggaga aactaaagcc ttattgagaa |
| 1801 | aacatgttat agatcctttt gttgctgaga gagtggaaca taggtcctag acagggtgaa |
| 1861 | gagttctggc acattttagc tgctactttg agacctcggt gatgttacct ggtgtggtca |
| 1921 | tcccatcttg tcctgtttta aggatatggg tggtgaaaga tgaaagaggc agagtttatc |
| 1981 | ccaatgactt ctctgtttga gttgggaagc ctcaccttca gacccagtaa ctgtccgcag |
| 2041 | ctgtctgcta gtggttgtct taacatcgta gtcctagttt gcatttttta aatcccctct |
| 2101 | gtttaaaagg tttgtaaaac aaaaacaaaa aactaagtct gctcagtgaa atgctgtaga |
| 2161 | accctaaata agtggtagaa gagtgtcact gaattttgtc tctgaattca gtataactga |
| 2221 | gttttgtcca tgctggtgtc tgggttatag gcctgatggg cctggtagtt ttccatcttg |
| 2281 | ttctggccta gaggtcagtc ctttgcactt cctcaaagct tgtgtacagt gctcacctaa |
| 2341 | atccatctga ctacttgttc ctgtgccctc ttgttttagg cctcgtttac ttttaaaaaa |
| 2401 | tgaaattgtt cattgctggg agaagaatgt tgtaattttt acttattaaa gtcaacttgt |
| 2461 | taagtttttt atgtattcct gttgggtttt cttgttgatc tcatgctagc agagcaaaaa |
| 2521 | ttgtaaaata ttttgattaa aaatctaggg acctttatgt cctatttgaa atgtgaaaaa |
| 2581 | aaaaaaaaaa aaa |

PUS1

| FEATURES | Location/Qualifiers |
|---|---|
| source | 1..1637 |
| | /organism = ″Homo sapiens″ |
| | /mol_type = ″mRNA″ |
| | /db_xref = ″taxon:9506″ |
| | /chromosome = ″12″ |
| | /map = ″12q24.33″ |
| gene | 1..1637 |
| | /gene = ″PUS1″ |
| | /gene_synonym = ″MLASA1″ |
| | /note = ″pseudouridylate synthase 1″ |
| | /db_xref = ″GeneID:80324″ |
| | /db_xref = ″HGNC:HGNC:15508″ |
| | /db_xref = ″MIM:608109″ |
| exon | 1..152 |
| | /gene = ″PUS1″ |
| | /gene_synonym = ″MLASA1″ |
| | /inference = ″alignment:Splign:2.1.0″ |
| misc_feature | 130..132 |
| | /gene = ″PUS1″ |
| | /gene_synonym = ″MLASA1″ |
| | /note = ″upstream in-frame stop codon″ |
| exon | 153..381 |
| | /gene = ″PUS1″ |
| | /gene_synonym = ″MLASA1″ |
| | /inference = ″alignment:Splign:2.1.0″ |
| CDS | 163..1362 |
| | /gene = ″PUS1″ |
| | /gene_synonym = ″MLASAl″ |
| | /EC_number = ″5.4.99.12″ |
| | /note = ″isoform 2 is encoded by transcript variant 2; |
| | tRNA |
| | uridine isomerase I; tRNA pseudouridine synthase A, |
| | mitochondrial; mitochondrial tRNA pseudouridine |
| | synthase |
| | A; tRNA pseudouridylate synthase I; tRNA |
| | pseudouridine(38-40) synthase″ |
| | /codon_start = 1 |
| | /product = ″tRNA pseudouridine synthase A isoform 2″ |
| | /protein_id = ″NP_001002019.1″ |
| | /db_xref = ″CCDS:CCDS319213.1″ |
| | /db_xref = ″GeneID:80324″ |
| | /db_xref = ″HGNC:HGNC:15508″ |
| | /db_xref = ″MIM:608109″ |

/transiation = ″MAGNAEPPPAGAACPQDRRSCSGRAGGDRVWEDGEHPAKKLKSG
GDEERREKPPKRKIVLLMAYSGKGYHGMQRNVGSSQFKTIEDDLVSALVRSGCIPENH
GEDMRKMSFQRCARTDKGVSAAGQVVSLKVWLIDDILEKINSHLPSHIRILGLKRVTG
GFNSKNRCDARTYCYLLPTFAFAHKDRDVQDETYRLSAETLQQVNRLLACYKGTHNFH
NFTSQKGPQDPSACRYILEMYCEEPFVREGLEFAVIRVKGQSFMMHQIRKMVGLVVAI
VKGYAPESVLERSWGTEKVDVPKAPGLGLVLERVHFEKYNQRFGNDGLHEPLDWAQEE
GKVAAFKEEHIYPTIIGTERDERSMAQWLSTLPIHNFSATALTAGGTGAKVPSPLEGS
EGDGDTD″

| | |
|---|---|
| exon | 382..519 |
| | /gene = ″PUS1″ |
| | /gene_synonym = ″MLASA1″ |
| | /Inference = ″alignment:Splign:2.1.0″ |
| exon | 520..622 |
| | /gene = ″PUS1″ |
| | /gene_synonym = ″MLASA1″ |
| | /Inference = ″alignment:Splign:2.1.0″ |
| exon | 623..1314 |
| | /gene = ″PUS1″ |
| | /gene synonym = ″MLASA1″ |
| | /Inference = ″alignment:Splign:2.1.0″ |
| exon | 1315..1637 |
| | /gene = ″PUS1″ |
| | /gene_synonym = ″MLASA1″ |
| | /Inference = ″alignment:Splign:2.1.0″ |
| STS | 1352..1510 |
| | /gene = ″PUS1″ |
| | /gene_synonym = ″MLASA1″ |
| | /standard_name = ″RH44488″ |
| | /db_xref = ″UnISTS:7173″ |
| regulatory | 1606..1611 |
| | /regulatory_class = ″polyA_signal_sequence″ |
| | /gene = ″PUS1″ |
| | /gene_synonym = ″MLASA1″ |
| polyA_site | 1635 |
| | /gene = ″PUS1″ |
| | /gene_synonym = ″MLASA1″ |

ORIGIN

| | |
|---|---|
| 1 | cccacgtggt ccggctccgg ctcagtcagc cgcgtcgcga atggggcagg agcgagcctc |
| 61 | tctggtcccg acgcgggtgg cccgggtctc ctcgactcct gaggaaagcc caccgggcgg |
| 121 | ggcgggaggt gaagaggctg gggaagtcag agctcgccgc gcatggccgg gaacgcggag |
| 181 | ccgccgcccg ccggagccgc atgcccccag gaccggaggt cctgcagcgg ccgggccggg |
| 241 | ggcgaccgcg tctgggagga cggagaacat ccggcgaaga agctcaagag cggtggcgac |
| 301 | gaggagcggc gcgagaagcc gcccaagcgg aagatcgtgc tgctcatggc ctattcgggc |
| 361 | aagggctacc acggcatgca gaggaatgtc gggtcctcac aattcaaaac aattgaagat |
| 421 | gacttggtgt ccgccctcgt ccggtcaggc tgtattcctg aaaatcatgg tgaggacatg |
| 481 | aggaaaatgt ccttccagcg ctgcgcccgg acagacaagg gtgtgtccgc agccggccag |
| 541 | gtggtatccc tgaaggtgtg gctgattgac gacattctag aaaagatcaa cagccacctt |
| 601 | ccctctcaca ttcggattct gggactgaag cgggtcacgg gcgggtttaa ctccaagaac |
| 661 | agatgtgatg ccaggaccta ttgctacctg ctgcccacgt ttgcctttgc gcacaaggac |
| 721 | cgggacgttc aggatgagac ctaccgcctg agcgccgaga cgctgcagca ggtcaacagg |
| 781 | ctcctggcct gctacaaggg cacgcacaac ttccacaatt tcacctcgca gaaggggccg |
| 841 | caggatccca gtgcctgccg ctacatcctg gagatgtact gcgaggaacc ctttgtgcgg |
| 901 | gagggcctgg agtttgcggt gatcagggtg aagggccaga gcttcatgat gcatcagatc |
| 961 | cggaagatgg tcggcctggt ggtggccatt gtgaagggtt atgcccctga gagcgtgctg |
| 1021 | gagcgcagct ggggcacaga gaaggtggac gtgcccaagg cgcccggact cggcctggtc |
| 1081 | ctggagaggg tgcacttcga gaagtacaac cagcgctttg gcaacgatgg gctgcatgag |
| 1141 | ccgctggact gggcgcagga ggaaggaaag gtcgcagcct tcaaggagga gcacatctac |
| 1201 | cccaccatca tcggcaccga gcgggacgaa cgctccatgg cccagtggct gagcaccttg |
| 1261 | cccatccaca acttcagtgc caccgctctc acggcaggtg gcacgggcgc caaggtgccc |
| 1321 | agtcccctgg aaggcagtga aggggacgga gacactgact gaggcgatgg gagctgccca |
| 1381 | ccagagtgcc tctgagcagc tcacagtgtg tgcccagatg tgccacccct gtgggcagca |
| 1441 | agaagctggg atcgctgcag ccatgttttc ccggccatgc cggcgttgta acctcaggac |
| 1501 | cttcccttgt aggaacagcc tttctcgaat ctgttttcag ctcttgcatt gcatagatga |
| 1561 | acctcagcat gtaaagaact atttttttaa agaagtgatt ttcttattaa acaagtacaa |
| 1621 | attttgctta gtcaatc |

PUS3

| FEATURES | Location/Qualifiers |
|---|---|
| source | 1..1862 |
| | /organism = ″Homo sapiens″ |
| | /mol_type = ″mRNA″ |
| | /db_xref = ″taxon:9606″ |
| | /chromosome = ″11″ |
| | /map = ″11q24.2″ |
| gene | 1..1862 |
| | /gene = ″PUS3″ |
| | /gene_synonym = ″2610020J05Rik; FKSG32; MRT55″ |
| | /note = ″pseudouridylate synthase 3″ |
| | /db_xref = ″GeneID:83480″ |
| | /db_xref = ″HGNC:HGNC:25461″ |
| | /db_xref = ″MIM:616283″ |
| exon | 1..52 |
| | /gene = ″PUS3″ |
| | /gene_synonym = ″2610020J05Rik; FKSG32; MRT55″ |
| | /inference = ″alignment:Splign:2.1.0″ |
| exon | 53..476 |
| | /gene = ″PUS3″ |
| | /gene_synonym = ″2610020J05Rik; FKSG32; MRT55″ |
| | /inference = ″alignment:Splign:2.1.0″ |
| CDS | 99..1544 |
| | /gene = ″PUS3″ |
| | /gen_synonym = ″2610020J05Rik; FKSG32; MRT55″ |
| | /EC_number = ″5.4.99.45″ |
| | /note = ″isoform 1 is encoded by transcript variant 1; |
| | tRNA |
| | pseudouridylate synthase 3; tRNA-uridine isomerase 3; |
| | tRNA |
| | pseudouridine synthase 3; tRNA pseudouridine(38/39) |
| | synthase″ |
| | /codon_start = 1 |
| | /product = ″tRNA pseudouridine(38/39) synthase isoform |
| | 1″ |
| | /protein_id = ″NP_112597.3″ |
| | /db_xref = ″CCDS:CCDS8466.1″ |
| | /db_xref = ″GeneID:83480″ |
| | /db_xref = ″HGNC:HGNC:25461″ |
| | /db_xref = ″MIM:616283″ |

/translation = ″MADNDTDRNQTEKLLKRVRELEQEVQRLKKEQAKNKEDSNIREN
aAGAGKTKRAFDFSAHGRRHVALRIAYMGWGYQGFASQENTNNTIEEKLFEALTKTRL
VESRQTSNYHRCGRTDKGVSAFGQVISLDLRSQFPRGRDSEDFNVKEEANAAAEEIRY
THILNRVLPPDIRILAWAPVEPSFSARFSCLERTYRYFFPRADLDIVTMDYAAQKYVG
THDFRNLCKMDVANGVINFQRTILSAQVQLVGQSPGEGRWQEPFQLCQFEVTGQAFLY
HQVRCMMAILFLIGQGMEKPEIIDELLNIEKNPQKPQYSMAVEFPLVLYDCKFENVKW
IYDQEAQEFNITHLQQLWANHAVKTHMLYSMLQGLDTVPVPCGIGPKMDGMTEWGNVK
PSVIKQTSAFVEGVKMRTYKPLMDRPKCQGLESRIQHFVRRGRIEHPHLFHEEETKAK
RDCNDTLEEENTNLETPTKRVCVDTEIKSII″

| | |
|---|---|
| misc_feature | 102..104 |
| | /gene = ″PUS3″ |
| | /gene_synonym = ″2610020J05Rik; FKSG32; MRT55″ |
| | /experiment = ″experimental evidence, no additional |
| | details |
| | recorded″ |
| | /note = ″N-acetylalanine. {ECO:0000244\|PubMed:19413330}; |
| | propagated from UniProtKB/Swiss-Prot (Q9BZE2.3); |
| | acetylation site″ |
| misc_feature | 1464..1466 |
| | /gene = ″PUS3″ |
| | /gene_synonym = ″2610020J05Rik; FKSG32; MRT55″ |
| | /experiment = ″experimental evidence, no additional |
| | details |
| | recorded″ |
| | /note = ″Phosphothreonine. |
| | {ECO:0000244\|PubMed:18669648}; |
| | propagated from UniProtKB/Swiss-Prot (Q9BZE2.3); |
| | phosphorylation site″ |
| misc_feature | 1494..1496 |
| | /gene = ″PUS3″ |
| | /gene_synonym = ″2610020J05Rik; FKSG32; MRT55″ |
| | /experiment = ″experimental evidence, no additional |
| | details |
| | recorded″ |
| | /note = ″Phosphothreonine. |
| | {ECO:0000244\|PubMed:23186163}; |
| | propagated from UniProtKB/Swiss-Prot (Q9BZE2.3); |
| | phosphorylation site″ |
| misc_feature | 1500..1502 |
| | /gene = ″PUS3″ |
| | /gene_synonym = ″2610020J05Rik; FKSG32; MRT55″ |
| | /experiment = ″experimental evidence, no additional |
| | details |
| | recorded″ |
| | /note = ″Phosphothreonine. |
| | {ECO:0000244\|PubMed:18669648}; |
| | propagated from UniProtKB/Swiss-Prot (Q9BZE2.3); |
| | phosphorylation site″ |
| exon | 477..1042 |
| | /gene = ″PUS3″ |
| | /gene_synonym = ″2610020J05Rik; FKSG32; MRT55″ |
| | /inference = ″alignment:Splign:2.1.0″ |
| STS | 732..892 |
| | /gene = ″PUS3″ |
| | /gene_synonym = ″2610020J05Rik; FKSG32; MRT55″ |
| | /standard_name = ″RH47976″ |
| | /db_xref = ″UniSTS:47549″ |
| exon | 1043..1844 |
| | /gene = ″PUS3″ |
| | /gene_synonym = ″2610020J05Rik; FKSG32; MRT55″ |
| | /inference = ″alignment:Splign:2.1.0″ |
| regutatory | 1822..1827 |
| | /regulatory_class = ″polyA_signal_sequence″ |
| | /gene = ″PUS3″ |
| | /gene_synonym = ″2610020J05Rik; FKSG32; MRT55″ |
| polA_site | 1844 |
| | /gene = ″PUS3″ |
| | /gene_synonym = ″2610020J05Rik; FKSG32; MRT55″ |

ORIGIN

| | |
|---|---|
| 1 | gcacagtgac agcttccttt ctcggaaacg cggcgcggcc ggctgccgga aaacagggca |
| 61 | gacctgtatg gttcgtttat tcctggggtt gtcatatcat ggctgataat gacacagaca |
| 121 | gaaaccagac tgagaagctc ctaaaaagag tacgagaact ggagcaagag gtgcaaagac |
| 181 | ttaaaaagga acaggccaaa aataaggagg actcaaacat tagagaaaat tcagcaggag |
| 241 | ctggaaaaac taagcgtgca tttgatttca gtgctcatgg ccgaagacac gtagccctaa |
| 301 | gaatagccta tatgggctgg ggataccagg gctttgctag tcaggaaaac acaaataata |
| 361 | ccattgaaga gaaactgttt gaagctctaa ccaagactcg actagtagaa agcagacaga |
| 421 | catccaacta tcaccgatgt gggagaacag ataaaggagt tagtgccttt ggacaggtga |
| 481 | tctcacttga ccttcgctct cagtttccaa ggggcaggga ttccgaggac tttaatgtaa |
| 541 | aagaggaggc taatgctgct gctgaagaga tccgttatac ccacattctc aatcgggtac |
| 601 | tccctccaga catccgtata ttggcctggg cccctgtaga accaagcttc agtgctaggt |
| 661 | tcagctgcct tgagcggact taccgctatt ttttccctcg tgctgattta gatattgtaa |
| 721 | ccatggatta tgcagctcag aagtatgttg gcacccatga tttcaggaac ttgtgtaaaa |
| 781 | tggatgtagc caacggtgtg attaattttc agaggactat tctatctgct caagtacagc |
| 841 | tagtgggcca gagcccaggt gaggggagat ggcaagaacc tttccagtta tgtcagtttg |
| 901 | aagtgactgg ccaggcattc ctttatcatc aagtccgatg tatgatggct atcctctttc |
| 961 | tgattggcca aggaatggag aagccagaga ttattgatga gctgctgaat atagagaaaa |
| 1021 | atccccaaaa gcctcaatat agtatggctg tagaatttcc tctagtctta tatgactgta |
| 1081 | agtttgaaaa tgtcaagtgg atctatgacc aggaggctca ggagttcaat attacccacc |
| 1141 | tacaacaact gtgggctaat catgctgtca aaactcacat gttgtatagt atgctacaag |
| 1201 | gactggacac tgttccagta ccctgtggaa taggaccaaa gatggatgga atgacagaat |
| 1261 | ggggaaatgt taagccctct gtcataaagc agaccagtgc ctttgtagaa ggagtgaaga |
| 1321 | tgcgcacata taagcccctc atggaccgtc ctaaatgcca aggactggaa tcccggatcc |
| 1381 | agcattttgt acgtagggga cgaattgagc acccacattt attccatgag gaagaaacaa |
| 1441 | aagccaaaag ggactgtaat gacacactag aggaagagaa tactaatttg gagacaccaa |
| 1501 | cgaagagggt ctgtgttgac acagaaatta aaagcatcat ttaaccatag acaatttgcc |
| 1561 | aggatctagg aaccacctaa tggtaggtgg acagaaaagg aaaaaaaaaa aaatttactt |
| 1621 | gcaagtacta ggaattcaga tgatcagctc ttaaaagaaa aaaaaaagca aaaagactaa |
| 1681 | agccctatta aggaagttat tgctttaata agaaatttca aatattctct tatcccggtc |
| 1741 | caaaaggatt aagcgattaa agaacgtaaa atggagatgt atttacatac acctggaaac |
| 1801 | ctgtgccttg tattcaaatt cattaaagcc taatcctgca agtaaaaaaa aaaaaaaaaa |
| 1861 | aa |

PUS7

| FEATURES | Location/Qualifiers |
|---|---|
| source | 1..3316 |
| | /organism = ″Homo sapiens″ |
| | /mol_type = ″mRNA″ |
| | /db_xref = ″taxon:9606″ |
| | /chromosome = ″7″ |
| | /map = ″7q22.3″ |
| gene | 1..3316 |
| | /gene = ″PUS7″ |
| | /note = ″pseudouridylate synthase 7″ |
| | /db_xref = ″GeneID:54517″ |
| | /db_xref = ″HGNC:HGNC:26033″ |
| | /db_xref = ″MIM:616261″ |
| exon | 1..406 |
| | /gene = ″PUS7″ |
| | /inference = ″alignment:Splign:2.1.0″ |
| CDS | 9..2012 |
| | /gene = ″PUS7″ |
| | /EC_number = ″4.2,1.70″ |
| | /note = ″isoform a is encoded by transcript variant 1; |
| | pseudouridylate synthase 7 homolog; pseudouridylate |
| | synthase 7 (putative)″ |
| | /codon_start = 1 |
| | /product = ″pseudouridylate synthase 7 homolog isoform |
| | a″ |
| | /protein_id = ″NP_001305092.1″ |
| | /db_xref = ″GeneID:54517″ |
| | /db_xref = ″HGNC:HGNC:26033″ |
| | /db_xref = ″MIM:616261″ |

/translation = ″MEMTEMTGVSLKRGALVVEDNDSGVPVEETKKQKLSECSLTKGQ
DGLQNDFLSISEDVPRPPDTVSTGKGGKNSEAQLEDEEEEEEDGLSEECEEEESESFA
DMMKHGLTEADVGITKEVSSHQGFSGILKERYSDFVVHEIGKDGRISHLNDLSIPVDE
EDPSEDIFTVLTAEEKQRLEELQLFKNKETSVAIEVIEDTKEKRTIIHQAIKSLFPGL
ETKTEDREGKKYIVAYHAAGKKALAKVRTAADPRKHSWPKSRGSYCHFVLYKENKDTM
DAINVLSKYLRVKPNIFSYMGTKDKRAITVQEIAVLKITAQRLAHLNKCLMNFKLGNF
SYQKNPLKLGELQGNHFTVVLRNITGTDDQVQQAMNSLKEIGFINYYGMQRFGTTAVP
TYQVGRAILQNSWTEVMDLILKPRSGAEKGYLVKCREEWAKTKDPTAALRKLPVKRCV
EGQLLRGLSKYGMKNIVSAFGIIPRNNRLMYIHSYQSYVWNNMVSKRIEDYGLKPVPG
DLVLKGATATYIEEDDVNNYSIHDVVMPLPGFDVIYPKHKIQEAYREMLTADNLDIDN
MRHKIRDYSLSGAYRKIIIRPQNVSWEVVAYDDPKIPLFNTDVDNLEGKTPPVFASEG
KYRALKMDFSLPPSTYATMAIREVLKMDTSIKNQTQLNTTWLR″

| | |
|---|---|
| misc_feature | 9..11 |
| | /gene = ″PUS7″ |
| | /experiment = ″experimental evidence, no additional |
| | details |
| | recorded″ |
| | /note = ″N-acetylmethionine. |
| | {ECO:0000244\|PubMed:19413330, |
| | ECO:0000244\|PubMed:22814378}; propagated from |
| | UniProtKB/Swiss-Prot (Q96PZ0.2); acetylation site″ |
| misc_feature | 36..38 |
| | /gene = ″PUS7″ |
| | /experiment = ″experimental evidence, no additional |
| | details |
| | recorded″ |
| | /note = ″Phosphoserine. {ECO:0000244\|PubMed:23186163}; |
| | propagated from UniProtKB/Swiss-Prot (Q96PZ0.2); |
| | phosphorylation site″ |
| misc_feature | 387..389 |
| | /gene = ″PUS7″ |
| | /experiment = ″experimental evidence, no additional |
| | details |
| | recorded″ |
| | /note = ″Phosphoserine. {ECO:0000244\|PubMed:23186163}; |
| | propagated from UniProtKB/Swiss-Prot (Q96PZ0.2); |
| | phosphorylation site″ |
| misc_feature | 1854..1856 |
| | /gene = ″PUS7″ |
| | /experiment = ″experimental evidence, no additional |
| | details |
| | recorded″ |
| | /note = ″Phosphothreonine. +ECO:0000244\|PubMed:19690332, |
| | ECO:0000244\|PubMed:23186163}; propagated from |
| | UniProtKB/Swiss-Prot (Q96PZ0.2); phosphorylation site″ |
| exon | 407..491 |
| | /gene = ″PUS7″ |
| | /inference = ″alignment:Splign:2.1.0″ |
| exon | 492..593 |
| | /gene = ″PUS7″ |
| | /inference = ″alignment:Splign:2.1.0″ |
| exon | 594..738 |
| | /gene = ″PUS7″ |
| | /inference = ″alignment:Splign:2.1.0″ |
| exon | 739..756 |
| | /gene = ″PUS7″ |
| | /inference = ″alignment:Splign:2.1.0″ |
| exon | 757..868 |
| | /gene = ″PUS7″ |
| | /inference = ″alignment:Splign:2.1.0″ |
| exon | 869..946 |
| | /gene = ″PUS7″ |
| | /inference = ″alignment:Splign:2.1.0″ |
| exon | 947..1075 |
| | /gene = ″PUS7″ |
| | /inference = ″alignment:Splign:2.1.0″ |
| exon | 1076..1201 |
| | /gene = ″PUS7″ |
| | /inference = ″alignment:Splign:2.1.0″ |
| exon | 1202..1263 |
| | /gene = ″PUS7″ |
| | /inference = ″alignment:Splign:2.1.0″ |
| exon | 1264..1424 |
| | /gene = ″PUS7″ |
| | /inference = ″alignment:Splign:2.1.0″ |
| exon | 1425..1551 |
| | /gene = ″PUS7″ |
| | /inference = ″alignment:Splign:2.1.0″ |
| exon | 1552..1653 |
| | /gene = ″PUS7″ |
| | /inference = ″alignment:Splign:2.1.0″ |
| exon | 1654..1783 |
| | /gene = ″PUS7″ |
| | /inference = ″alignment:Splign:2.1.0″ |
| exon | 1784..1875 |
| | /gene = ″PUS7″ |
| | /inference = ″alignment:Splign:2.1.0″ |
| exon | 1876..3301 |
| | /gene = ″PUS7″ |
| | /inference = ″alignment:Splign:2.1.0″ |

ORIGIN

| | |
|---|---|
| 1 | ccttaaagat ggagatgaca gaaatgactg gtgtgtcgct gaaacgtggg gcactggttg |
| 61 | tcgaagataa tgacagtgga gtcccagttg aagagacaaa aaaacagaag ctgtcggaat |
| 121 | gcagtctaac caaaggtcaa gatgggctac agaatgactt tctgtccatc agtgaagacg |
| 181 | tgcctcggcc tcctgacact gtcagtactg ggaaaggtgg aaagaattct gaggctcagt |
| 241 | tggaagatga ggaagaagag gaggaagatg gactttcaga ggagtgcgag gaggaggaat |
| 301 | cagagagttt tgcagacatg atgaagcatg gactcactga ggctgacgta ggcatcacca |
| 361 | agtttgtgag ttctcatcaa gggttctcgg gaatcttaaa agaaagatac tccgacttcg |
| 421 | ttgttcatga aataggaaaa gatggacgga tcagccattt gaatgacttg tccattccag |
| 481 | tggatgagga ggacccttca gaagacatat ttacagtttt gacagctgaa gaaaagcagc |
| 541 | gattggaaga gctccagctg ttcaaaaata aggaaaccag tgttgccatt gaggttatcg |
| 601 | aggacaccaa agagaaaaga accatcatcc atcaggctat caaatctctg tttccaggat |
| 661 | tagagacaaa aacagaggat agggagggga agaaatacat tgtagcctac cacgcagctg |
| 721 | ggaaaaaggc tttggcaaag gtcagaactg cagcagatcc aagaaaacat tcttggccaa |
| 781 | aatctagggg aagttactgc cacttcgtac tatataagga aaacaaagac accatggatg |
| 841 | ctattaatgt actctccaaa tacttaagag tcaagccaaa tatattctcc tacatgggaa |
| 901 | ccaaagataa aagggctata acagttcaag aaattgctgt tctcaaaata actgcacaaa |
| 961 | gacttgccca cctgaataag tgcttgatga actttaagct agggaatttc agctatcaaa |
| 1021 | aaaacccact gaaattggga gagcttcaag gaaaccactt cactgttgtt ctcagaaata |
| 1081 | taacaggaac tgatgaccaa gtacagcaag ctatgaactc tctcaaggag attggattta |
| 1141 | ttaactacta tggaatgcaa agatttggaa ccacagctgt ccctacgtat caggttggaa |
| 1201 | gagctatact acaaaattcc tggacagaag tcatggattt aatattgaaa ccccgctctg |
| 1261 | gagctgaaaa gggctacttg gttaaatgca gagaagaatg ggcaaagacc aaagacccaa |
| 1321 | ctgctgccct cagaaaacta cctgtcaaaa ggtgtgtgga agggcagctg cttcgaggac |
| 1381 | tttcaaaata tggaatgaag aatatagtct ctgcatttgg cataataccc agaaataatc |
| 1441 | gcttaatgta tattcatagc taccaaagct atgtgtggaa taacatggta agcaagagga |
| 1501 | tagaagacta tggactaaaa cctgttccag gggacctcgt tctcaaagga gccacagcca |
| 1561 | cctatattga ggaagatgat gttaataatt actctatcca tgatgtggta atgcccttgc |
| 1621 | ctggtttcga tgttatctac ccaaagcata aaattcaaga agcctacagg gaaatgctca |
| 1681 | cagctgacaa tcttgatatt gacaacatga gacacaaaat tcgagattat tccttgtcag |
| 1741 | gggcctaccg aaagatcatt attcgtcctc agaatgttag ctgggaagtc gttgcatatg |
| 1801 | atgatcccaa aattccactt ttcaacacag atgtggacaa cctagaaggg aagacaccac |
| 1861 | cagtttttgc ttctgaaggc aaatacaggg ctctgaaaat ggatttttct ctaccccctt |
| 1921 | ctacttacgc caccatggcc attcgagaag tgctaaaaat ggataccagt atcaagaacc |
| 1981 | agacgcagct gaatacaacc tggcttcgct gagcagtacc ttgtccacag attagaaaac |
| 2041 | gtacacaagt gtttgcttcc tggctccctg tgcatttttg tcttagttca gactcatata |
| 2101 | tggatttcaa atctttgtaa taaaaattat ttgtattttt aagtttttat tagcttaaag |
| 2161 | aaataatttg caatatttgt acatgtacac aaatcctgag gttcttaatt ttagctcaga |
| 2221 | atataaatta gtcaaaatac acttcaggtg cttaaatcag agtaaaatgt cagctttaca |
| 2281 | ataataaaaa aaggactttg gtttaaagta gcaggtttag gttttgctac attctcaaaa |
| 2341 | gacagcagga gtatttgaca catctgtgat ggagtataca acaatgcatt ttaagagcaa |
| 2401 | atgcaacaaa acaaatctgg actatggata aataatttga gagctgccac ccacaaatat |
| 2461 | aaatacagta ctcatgctga ctgaaataat aagacatcta caaatttata aacaaaaagt |
| 2521 | gattgtcatt atcctgctta tgtactagat tcaggcaagc attatagact ttttggttgc |
| 2581 | ggtggctttt gcatttatat tatcaatgcc ttgcaggaac gttgcattga taggcccatt |
| 2641 | ttattttttt attttttttt tcgagacagg atctcactct gtagcacagg ctggattgca |
| 2701 | gtgcaatcct gcaattctca atcttgcact gcagcctcga cctcccaggc tccagtgact |
| 2761 | ctcccacctc agcctcctaa gtagctggga gtacaggcgc gcaccaccac gcctagctga |
| 2821 | tttttgtatt tttttgtaga gacgggggtt tggccatgtt gccgaggcta actcctggga |
| 2881 | ttacaggcat gagctgtgct ggccgggttt ttttttcttg atgtaaacgt gtacagctgt |
| 2941 | tttattagtt aaggtctaat ttttactcta ggtgcctttt atgttcagaa ctctttccac |
| 3001 | tggactggta tttgctcaaa aataaataat ggtagagaag aaaactataa aaatggacaa |
| 3061 | ggctttcttc tatcagtagc gtttaccctt tgtcaccagt ggctttggta tttccatgtc |
| 3121 | tggcattgca taaacttctc tggtgtgaaa ggataaatat gcctttctaa agttgtatat |
| 3181 | caaaattgta tcaattttta ttttctatga tttctagaaa caaatgtaat aaatattttt |
| 3241 | aaaatctcct ttctactggt tatgtaaata aatcaaataa atatatcaaa atgagtgcag |
| 3301 | aaaaaaaaaa aaaaaa |

1. A fusion protein comprising:
(i) a guide nucleotide sequence-programmable RNA binding protein; and
(ii) an RNA pseudouridylation modification protein (RPMP).

2. The fusion protein of claim 1, wherein the guide nucleotide sequence-programmable RNA binding protein is selected from: Cas9, modified Cas9, Cas13a, Cas13b, CasRX/Cas13d, and a biological equivalent of each thereof.

3. The fusion protein of claim 2, wherein the guide nucleotide sequence-programmable RNA binding protein is selected from: Steptococcus pyogenes Cas9 (spCas9), Staphylococcus aureus Cas9 (saCas9), Francisella novicida Cas9 (FnCas9), Neisseria meningitidis Cas9 (nmCas9), Streptococcus thermophilus 1 Cas9 (St1Cas9), Streptococcus thermophilus 3 Cas9 (St3Cas9), Campylobacter jejuni Cas9 (CjeCas9), and Brevibacillus laterosporus Cas9 (BlatCas9).

4. (canceled)

5. The fusion protein of claim 1, further comprising a linker.

6. The fusion protein of claim 5, wherein the linker is a peptide linker.

7. (canceled)

8. The fusion protein of claim 5, wherein the linker is a non-peptide linker.

9.-11. (canceled)

12. The fusion protein of claim 1, wherein the guide nucleotide sequence-programmable RNA binding protein is bound to a guide RNA (gRNA), a crisprRNA (crRNA), or a trans-activating crRNA (tracrRNA).

13.-14. (canceled)

15. A polynucleotide encoding the fusion protein of claim 1.

16. A vector comprising the polynucleotide of claim 15, optionally wherein the vector is an adenoviral vector, an adeno-associated viral vector, or a lentiviral vector.

17. The vector of claim 16, further comprising an expression control element.

18. The vector of claim 16, further comprising a selectable marker.

19. The vector of claim 16, further comprising a polynucleotide encoding either (i) a gRNA, or (ii) a crRNA and a tracrRNA.

20.-23. (canceled)

24. A viral particle comprising the vector of claim 16.

25. A cell comprising the vector of claim 16.

26.-28. (canceled)

29. A system for modulating RNA pseudouridylation of a target RNA, the system comprising:
(i) a fusion protein comprising: (a) a guide nucleotide sequence-programmable RNA binding protein, and (b) an RNA pseudouridylation modification protein (RPMP); and
(ii) a gRNA; or
(iii) a crRNA and a tracrRNA;
wherein the gRNA or the crRNA comprises a sequence complementary to a target RNA, and optionally the gRNA or the crRNA comprises a mismatch at a uridine residue.

30.-34. (canceled)

35. A method for modulating RNA pseudouridylation of a target RNA, the method comprising contacting the target mRNA with the fusion protein of claim 1, wherein the guide nucleotide sequence-programmable RNA binding protein binds a gRNA or a crRNA that hybridizes to a region of the target RNA.

36. A method for preventing nonsense-mediated mRNA decay, the method comprising contacting a target mRNA with the fusion protein of claim 1, wherein the guide nucleotide sequence-programmable RNA binding protein binds a gRNA or a crRNA that hybridizes to a region of the target RNA.

37.-46. (canceled)

47. A method for treating a disease or condition associated with RNA pseudouridylation of a target RNA in a subject in need thereof, the method comprising administering a fusion protein comprising (i) a guide nucleotide sequence-programmable RNA binding protein, and (ii) an RNA pseudouridylation modification protein (RPMP), a polynucleotide encoding a fusion protein comprising (i) a guide nucleotide sequence-programmable RNA binding protein, and (ii) an RNA pseudouridylation modification protein (RPMP), a vector comprising a polypeptide encoding a fusion protein comprising (i) a guide nucleotide sequence-programmable RNA binding protein, and (ii) an RNA pseudouridylation modification protein (RPMP), a viral particle comprising a vector comprising a polypeptide encoding a fusion protein comprising (i) a guide nucleotide sequence-programmable RNA binding protein, and (ii) an RNA pseudouridylation modification protein (RPMP), or a cell comprising a vector comprising a polypeptide encoding a fusion protein comprising (i) a guide nucleotide sequence-programmable RNA binding protein, and (ii) an RNA pseudouridylation modification protein (RPMP) to the subject, thereby treating the disease or condition associated with RNA pseudouridylation.

48.-54. (canceled)

55. A kit comprising the fusion protein of claim 1 and optionally instructions for use.

56. (canceled)

57. A non-human transgenic animal comprising the fusion protein of claim 1.


SEQUENCE LISTING (31 sequences)

SEQ ID NO: 1 - protein, 1368 aa, Streptococcus pyogenes [sequence omitted]

SEQ ID NO: 2 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 3 - protein, 1121 aa, Streptococcus thermophilus
MSDLVLGLDIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINDKGKEVPCNPFLKYKEEHGYIRKYSKKGNGPEIKSLKYYDSKLGNHIDITPKDSNNKVVLQSVSPWRADVYFNKTTGKYEILGLKYADLQFDKGTGTYKISQEKYNDIKKKEGVDSDSEFKFTLYKNDLLLVKDTETKEQQLFRFLSRTMPKQKHYVELKPYDKQKFEGGEALIKVLGNVANSGQCKKGLGKSNISIYKVRTDVLGNQHIIKNEGDKPKLDF

SEQ ID NO: 4 - protein, 1082 aa, Neisseria meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 5 - protein, 1037 aa, Parvibaculum lavamentivorans
MERIFGFDIGTTSIGFSVIDYSSTQSAGNIQRLGVRIFPEARDPDGTPLNQQRRQKRMMRRQLRRRRIRRKALNETLHEAGFLPAYGSADWPVVMADEPYELRRRGLEEGLSAYEFGRAIYHLAQHRHFKGRELEESDTPDPDVDDEKEAANERAATLKALKNEQTTLGAWLARRPPSDRKRGIHAHRNVVAEEFERLWEVQSKFHPALKSEEMRARISDTIFAQRPVFWRKNTLGECRFMPGEPLCPKGSWLSQQRRMLEKLNNLAIAGGNARPLDAEERDAILSKLQQQASMSWPGVRSALKALYKQRGEPGAEKSLKFNLELGGESKLLGNALEAKLADMFGPDWPAHPRKQEIRHAVHERLWAADYGETPDKKRVIILSEKDRKAHREAAANSFVADFGITGEQAAQLQALKLPTGWEPYSIPALNLFLAELEKGERFGALVNGPDWEGWRRTNFPHRNQPTGEILDKLPSPASKEERERISQLRNPTVVRTQNELRKVVNNLIGLYGKPDRIRIEVGRDVGKSKREREEIQSGIRRNEKQRKKATEDLIKNGIANPSRDDVEKWILWKEGQERCPYTGDQIGFNALFREGRYEVEHIWPRSRSFDNSPRNKTLCRKDVNIEKGNRMPFEAFGHDEDRWSAIQIRLQGMVSAKGGTGMSPGKVKRFLAKTMPEDFAARQLNDTRYAAKQILAQLKRLWPDMGPEAPVKVEAVTGQVTAQLRKLWTLNNILADDGEKTRADHRHHAIDALTVACTHPGMTNKLSRYWQLRDDPRAEKPALTPPWDTIRADAEKAVSEIVVSHRVRKKVSGPLHKETTYGDTGTDIKTKSGTYRQFVTRKKIESLSKGELDEIRDPRIKEIVAAHVAGRGGDPKKAFPPYPCVSPGGPEIRKVRLTSKQQLNLMAQTGNGYADLGSNHHIAIYRLPDGKADFEIVSLFDASRRLAQRNPIVQRTRADGASFVMSLAAGEAIMIPEGSKKGIWIVQGVWASGQVVLERDTDADHSTTTRPMPNPILKDDAKKVSIDPIGRVRPSND

SEQ ID NO: 6 - protein, 1084 aa, Corynebacterium diphtheriae
MKYHVGIDVGTFSVGLAAIEVDDAGMPIKTLSLVSHIHDSGLDPDEIKSAVTRLASSGIARRTRRLYRRKRRRLQQLDKFIQRQGWPVIELEDYSDPLYPWKVRAELAASYIADEKERGEKLSVALRHIARHRGWRNPYAKVSSLYLPDGPSDAFKAIREEIKRASGQPVPETATVGQMVTLCELGTLKLRGEGGVLSARLQQSDYAREIQEICRMQEIGQELYRKIIDVVFAAESPKGSASSRVGKDPLQPGKNRALKASDAFQRYRIAALIGNLRVRVDGEKRILSVEEKNLVFDHLVNLTPKKEPEWVTIAEILGIDRGQLIGTATMTDDGERAGARPPTHDTNRSIVNSRIAPLVDWWKTASALEQHAMVKALSNAEVDDFDSPEGAKVQAFFADLDDDVHAKLDSLHLPVGRAAYSEDTLVRLTRRMLSDGVDLYTARLQEFGIEPSWTPPTPRIGEPVGNPAVDRVLKTVSRWLESATKTWGAPERVIIEHVREGFVTEKRAREMDGDMRRRAARNAKLFQEMQEKLNVQGKPSRADLWRYQSVQRQNCQCAYCGSPITFSNSEMDHIVPRAGQGSTNTRENLVAVCHRCNQSKGNTPFAIWAKNTSIEGVSVKEAVERTRHWVTDTGMRSTDFKKFTKAVVERFQRATMDEEIDARSMESVAWMANELRSRVAQHFASHGTTVRVYRGSLTAEARRASGISGKLKFFDGVGKSRLDRRHHAIDAAVIAFTSDYVAETLAVRSNLKQSQAHRQEAPQWREFTGKDAEHRAAWRVWCQKMEKLSALLTEDLRDDRVVVMSNVRLRLGNGSAHKETIGKLSKVKLSSQLSVSDIDKASSEALWCALTREPGFDPKEGLPANPERHIRVNGTHVYAGDNIGLFPVSAGSIALRGGYAELGSSFHHARVYKITSGKKPAFAMLRVYTIDLLPYRNQDLFSVELKPQTMSMRQAEKKLRDALATGNAEYLGWLVVDDELVVDTSKIATDQVKAVEAELGTIRRWRVDGFFSPSKLRLRPLQMSKEGIKKESAPELSKIIDRPGWLPAVNKLFSDGNVTVVRRDSLGRVRLESTAHLPVTWKVQ

SEQ ID NO: 7 - protein, 1130 aa, Streptococcus pasteurianus
MTNGKILGLDIGIASVGVGIIEAKTGKVVHANSRLFSAANAENNAERRGFRGSRRLNRRKKHRVKRVRDLFEKYGIVTDFRNLNLNPYELRVKGLTEQLKNEELFAALRTISKRRGISYLDDAEDDSTGSTDYAKSIDENRRLLKNKTPGQIQLERLEKYGQLRGNFTVYDENGEAHRLINVFSTSDYEKEARKILETQADYNKKITAEFIDDYVEILTQKRKYYHGPGNEKSRTDYGRFRTDGTTLENIFGILIGKCNFYPDEYRASKASYTAQEYNFLNDLNNLKVSTETGKLSTEQKESLVEFAKNTATLGPAKLLKEIAKILDCKVDEIKGYREDDKGKPDLHTFEPYRKLKFNLESINIDDLSREVIDKLADILTLNTEREGIEDAIKRNLPNQFTEEQISEIIKVRKSQSTAFNKGWHSFSAKLMNELIPELYATSDEQMTILTRLEKFKVNKKSSKNTKTIDEKEVTDEIYNPVVAKSVRQTIKIINAAVKKYGDFDKIVIEMPRDKNADDEKKFIDKRNKENKKEKDDALKRAAYLYNSSDKLPDEVFHGNKQLETKIRLWYQQGERCLYSGKPISIQELVHNSNNFEIDHILPLSLSFDDSLANKVLVYAWTNQEKGQKTPYQVIDSMDAAWSFREMKDYVLKQKGLGKKKRDYLLTTENIDKIEVKKKFIERNLVDTRYASRVVLNSLQSALRELGKDTKVSVVRGQFTSQLRRKWKIDKSRETYHHHAVDALIIAASSQLKLWEKQDNPMFVDYGKNQVVDKQTGEILSVSDDEYKELVFQPPYQGFVNTISSKGFEDEILFSYQVDSKYNRKVSDATIYSTRKAKIGKDKKEETYVLGKIKDIYSQNGFDTFIKKYNKDKTQFLMYQKDSLTWENVIEVILRDYPTTKKSEDGKNDVKCNPFEEYRRENGLICKYSKKGKGTPIKSLKYYDKKLGNCIDITPEESRNKVILQSINPWRADVYFNPETLKYELMGLKYSDLSFEKGTGNYHISQEKYDAIKEKEGIGKKSEFKFTLYRNDLILIKDIASGEQEIYRFLSRTMPNVNHYVELKPYDKEKFDNVQELVEALGEADKVGRCIKGLNKPNISIYKVRTDVLGNKYFVKKKGDKPKLDFKNNKK

SEQ ID NO: 8 - protein, 1082 aa, Neisseria cinerea
MAAFKPNPMNYILGLDIGIASVGWAIVEIDEEENPIRLIDLGVRVFERAEVPKTGDSLAAARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNTHALQTGDFRTPAELALNKFEKESGHIRNQRGDYSHTFNRKDLQAELNLLFEKQKEFGNPHVSDGLKEGIETLLMTQRPALSGDAVQKMLGHCTFEPTEPKAAKNTYTAERFVWLTKLNNLRILEQGSERPLTDTERATLMDEPYRKSKLTYAQARKLLDLDDTAFFKGLRYGKDNAEASTLMEMKAYHAISRALEKEGLKDKKSPLNLSPELQDEIGTAFSLFKTDEDITGRLKDRVQPEILEALLKHISFDKFVQISLKALRRIVPLMEQGNRYDEACTEIYGDHYGKKNTEEKIYLPPIPADEIRNPVVLRALSQARKVINGVVRRYGSPARIHIETAREVGKSFKDRKEIEKRQEENRKDREKSAAKFREYFPNFVGEPKSKDILKLRLYEQQHGKCLYSGKEINLGRLNEKGYVEIDHALPFSRTWDDSFNNKVLALGSENQNKGNQTPYEYFNGKDNSREWQEFKARVETSRFPRSKKQRILLQKFDEDGFKERNLNDTRYINRFLCQFVADHMLLTGKGKRRVFASNGQITNLLRGFWGLRKVRAENDRHHALDAVVVACSTIAMQQKITRFVRYKEMNAFDGKTIDKETGEVLHQKAHFPQPWEFFAQEVMIRVFGKPDGKPEFEEADTPEKLRTLLAEKLSSRPEAVHKYVTPLFISRAPNRKMSGQGHMETVKSAKRLDEGISVLRVPLTQLKLKDLEKMVNREREPKLYEALKARLEAHKDDPAKAFAEPFYKYDKAGNRTQQVKAVRVEQVQKTGVWVHNHNGIADNATIVRVDVFEKGGKYYLVPIYSWQVAKGILPDRAVVQGKDEEDWTVMDDSFEFKFVLYANDLIKLTAKKNEFLGYFVSLNRATGAIDIRTHDTDSTKGKNGIFQSVGVKTALSFQKYQIDELGKEIRPCRLKKRPPVR

SEQ ID NO: 9 - protein, 1003 aa, Campylobacter lari
MRILGFDIGINSIGWAFVENDELKDCGVRIFTKAENPKNKESLALPRRNARSSRRRLKRRKARLIAIKRILAKELKLNYKDYVAADGELPKAYEGSLASVYELRYKALTQNLETKDLARVILHIAKHRGYMNKNEKKSNDAKKGKILSALKNNALKLENYQSVGEYFYKEFFQKYKKNTKNFIKIRNTKDNYNNCVLSSDLEKELKLILEKQKEFGYNYSEDFINEILKVAFFQRPLKDFSHLVGACTFFEEEKRACKNSYSAWEFVALTKIINEIKSLEKISGEIVPTQTINEVLNLILDKGSITYKKFRSCINLHESISFKSLKYDKENAENAKLIDFRKLVEFKKALGVHSLSRQELDQISTHITLIKDNVKLKTVLEKYNLSNEQINNLLEIEFNDYINLSFKALGMILPLMREGKRYDEACEIANLKPKTVDEKKDFLPAFCDSIFAHELSNPVVNRAISEYRKVLNALLKKYGKVHKIHLELARDVGLSKKAREKIEKEQKENQAVNAWALKECENIGLKASAKNILKLKLWKEQKEICIYSGNKISIEHLKDEKALEVDHIYPYSRSFDDSFINKVLVFTKENQEKLNKTPFEAFGKNIEKWSKIQTLAQNLPYKKKNKILDENFKDKQQEDFISRNLNDTRYIATLIAKYTKEYLNFLLLSENENANLKSGEKGSKIHVQTISGMLTSVLRHTWGFDKKDRNNHLHHALDAIIVAYSTNSIIKAFSDFRKNQELLKARFYAKELTSDNYKHQVKFFEPFKSFREKILSKIDEIFVSKPPRKRARRALHKDTFHSENKIIDKCSYNSKEGLQIALSCGRVRKIGTKYVENDTIVRVDIFKKQNKFYAIPIYAMDFALGILPNKIVITGKDKNNNPKQWQTIDESYEFCFSLYKNDLILLQKKNMQEPEFAYYNDFSISTSSICVEKHDNKFENLTSNQKLLFSNAKEGSVKVESLGIQNLKVFEKYIITPLGDKIKADFQPRENISLKTSKKYGLR

SEQ ID NO: 10 - protein, 1395 aa, Treponema denticola
MKKEIKDYFLGLDVGTGSVGWAVTDTDYKLLKANRKDLWGMRCFETAETAEVRRLHRGARRRIERRKKRIKLLQELFSQEIAKTDEGFFQRMKESPFYAEDKTILQENTLFNDKDFADKTYHKAYPTINHLIKAWIENKVKPDPRLLYLACHNIIKKRGHFLFEGDFDSENQFDTSIQALFEYLREDMEVDIDADSQKVKEILKDSSLKNSEKQSRLNKILGLKPSDKQKKAITNLISGNKINFADLYDNPDLKDAEKNSISFSKDDFDALSDDLASILGDSFELLLKAKAVYNCSVLSKVIGDEQYLSFAKVKIYEKHKTDLTKLKNVIKKHFPKDYKKVFGYNKNEKNNNNYSGYVGVCKTKSKKLIINNSVNQEDFYKFLKTILSAKSEIKEVNDILTEIETGTFLPKQISKSNAEIPYQLRKMELEKILSNAEKHFSFLKQKDEKGLSHSEKIIMLLTFKIPYYIGPINDNHKKFFPDRCWVVKKEKSPSGKTTPWNFFDHIDKEKTAEAFITSRTNFCTYLVGESVLPKSSLLYSEYTVLNEINNLQIIIDGKNICDIKLKQKIYEDLFKKYKKITQKQISTFIKHEGICNKTDEVIILGIDKECTSSLKSYIELKNIFGKQVDEISTKNMLEEIIRWATIYDEGEGKTILKTKIKAEYGKYCSDEQIKKILNLKFSGWGRLSRKFLETVTSEMPGFSEPVNIITAMRETQNNLMELLSSEFTFTENIKKINSGFEDAEKQFSYDGLVKPLFLSPSVKKMLWQTLKLVKEISHITQAPPKKIFIEMAKGAELEPARTKTRLKILQDLYNNCKNDADAFSSEIKDLSGKIENEDNLRLRSDKLYLYYTQLGKCMYCGKPIEIGHVFDTSNYDIDHIYPQSKIKDDSISNRVLVCSSCNKNKEDKYPLKSEIQSKQRGFWNFLQRNNFISLEKLNRLTRATPISDDETAKFIARQLVETRQATKVAAKVLEKMFPETKIVYSKAETVSMFRNKFDIVKCREINDFHHAHDAYLNIVVGNVYNTKFTNNPWNFIKEKRDNPKIADTYNYYKVFDYDVKRNNITAWEKGKTIITVKDMLKRNTPIYTRQAACKKGELFNQTIMKKGLGQHPLKKEGPFSNISKYGGYNKVSAAYYTLIEYEEKGNKIRSLETIPLYLVKDIQKDQDVLKSYLTDLLGKKEFKILVPKIKINSLLKINGFPCHITGKTNDSFLLRPAVQFCCSNNEVLYFKKIIRFSEIRSQREKIGKTISPYEDLSFRSYIKENLWKKTKNDEIGEKEFYDLLQKKNLEIYDMLLTKHKDTIYKKRPNSATIDILVKGKEKFKSLIIENQFEVILEILKLFSATRNVSDLQHIGGSKYSGVAKIGNKISSLDNCILIYQSITGIFEKRIDLLKV

SEQ ID NO: 11 - protein, 1345 aa, Streptococcus mutans
MKKPYSIGLDIGTNSVGWAVVTDDYKVPAKKMKVLGNTDKSHIEKNLLGALLFDSGNTAEDRRLKRTARRRYTRRRNRILYLQEIFSEEMGKVDDSFFHRLEDSFLVTEDKRGERHPIFGNLEEEVKYHENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEELEVLLAQIGDNYAELFLSAKKLYDSILLSGILTVTDVGTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKLLTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDHIIPQAFIKDNSIDNRVLTSSKENRGKSDDVPSKDVVRKMKSYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHKENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLNKLGGD

SEQ ID NO: 12 - protein, 1388 aa, Streptococcus thermophilus
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 13 - protein, 984 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 14 - protein, 1056 aa, Pasteurella multocida
MQTTNLSYILGLDLGIASVGWAVVEINENEDPIGLIDVGVRIFERAEVPKTGESLALSRRLARSTRRLIRRRAHRLLLAKRFLKREGILSTIDLEKGLPNQAWELRVAGLERRLSAIEWGAVLLHLIKHRGYLSKRKNESQTNNKELGALLSGVAQNHQLLQSDDYRTPAELALKKFAKEEGHIRNQRGAYTHTFNRLDLLAELNLLFAQQHQFGNPHCKEHIQQYMTELLMWQKPALSGEAILKMLGKCTHEKNEFKAAKHTYSAERFVWLTKLNNLRILEDGAERALNEEERQLLINHPYEKSKLTYAQVRKLLGLSEQAIFKHLRYSKENAESATFMELKAWHAIRKALENQGLKDTWQDLAKKPDLLDEIGTAFSLYKTDEDIQQYLTNKVPNSVINALLVSLNFDKFIELSLKSLRKILPLMEQGKRYDQACREIYGHHYGEANQKTSQLLPAIPAQEIRNPVVLRTLSQARKVINAIIRQYGSPARVHIETGRELGKSFKERREIQKQQEDNRTKRESAVQKFKELFSDFSSEPKSKDILKFRLYEQQHGKCLYSGKEINIHRLNEKGYVEIDHALPFSRTWDDSFNNKVLVLASENQNKGNQTPYEWLQGKINSERWKNFVALVLGSQCSAAKKQRLLTQVIDDNKFIDRNLNDTRYIARFLSNYIQENLLLVGKNKKNVFTPNGQITALLRSRWGLIKARENNNRHHALDAIVVACATPSMQQKITRFIRFKEVHPYKIENRYEMVDQESGEIISPHFPEPWAYFRQEVNIRVFDNHPDTVLKEMLPDRPQANHQFVQPLFVSRAPTRKMSGQGHMETIKSAKRLAEGISVLRIPLTQLKPNLLENMVNKEREPALYAGLKARLAEFNQDPAKAFATPFYKQGGQQVKAIRVEQVQKSGVLVRENNGVADNASIVRTDVFIKNNKFFLVPIYTWQVAKGILPNKAIVAHKNEDEWEEMDEGAKFKFSLFPNDLVELKTKKEYFFGYYIGLDRATGNISLKEHDGEISKGKDGVYRVGVKLALSFEKYQVDELGKNRQICRPQQRQPVR

SEQ ID NO: 15 - protein, 1629 aa, Francisella novicida
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 16 - protein, 1371 aa, Lactobacillus buchneri
MKVNNYHIGLDIGTSSIGWVAIGKDGKPLRVKGKTAIGARLFQEGNPAADRRMFRTTRRRLSRRKWRLKLLEEIFDPYITPVDSTFFARLKQSNLSPKDSRKEFKGSMLFPDLTDMQYHKNYPTIYHLRHALMTQDKKFDIRMVYLAIHHIVKYRGNFLNSTPVDSFKASKVDFVDQFKKLNELYAAINPEESFKINLANSEDIGHQFLDPSIRKFDKKKQIPKIVPVMMNDKVTDRLNGKIASEIIHAILGYKAKLDVVLQCTPVDSKPWALKFDDEDIDAKLEKILPEMDENQQSIVAILQNLYSQVTLNQIVPNGMSLSESMIEKYNDHHDHLKLYKKLIDQLADPKKKAVLKKAYSQYVGDDGKVIEQAEFWSSVKKNLDDSELSKQIMDLIDAEKFMPKQRTSQNGVIPHQLHQRELDEIIEHQSKYYPWLVEINPNKHDLHLAKYKIEQLVAFRVPYYVGPMITPKDQAESAETVFSWMERKGTETGQITPWNFDEKVDRKASANRFIKRMTTKDTYLIGEDVLPDESLLYEKFKVLNELNMVRVNGKLLKVADKQAIFQDLFENYKHVSVKKLQNYIKAKTGLPSDPEISGLSDPEHFNNSLGTYNDFKKLFGSKVDEPDLQDDFEKIVEWSTVFEDKKILREKLNEITWLSDQQKDVLESSRYQGWGRLSKKLLTGIVNDQGERIIDKLWNTNKNFMQIQSDDDFAKRIHEANADQMQAVDVEDVLADAYTSPQNKKAIRQVVKVVDDIQKAMGGVAPKYISIEFTRSEDRNPRRTISRQRQLENTLKDTAKSLAKSINPELLSELDNAAKSKKGLTDRLYLYFTQLGKDIYTGEPINIDELNKYDIDHILPQAFIKDNSLDNRVLVLTAVNNGKSDNVPLRMFGAKMGHFWKQLAEAGLISKRKLKNLQTDPDTISKYAMHGFIRRQLVETSQVIKLVANILGDKYRNDDTKIIEITARMNHQMRDEFGFIKNREINDYHHAFDAYLTAFLGRYLYHRYIKLRPYFVYGDFKKFREDKVTMRNFNFLHDLTDDTQEKIADAETGEVIWDRENSIQQLKDVYHYKFMLISHEVYTLRGAMFNQTVYPASDAGKRKLIPVKADRPVNVYGGYSGSADAYMAIVRIHNKKGDKYRVVGVPMRALDRLDAAKNVSDADFDRALKDVLAPQLTKTKKSRKTGEITQVIEDFEIVLGKVMYRQLMIDGDKKFMLGSSTYQYNAKQLVLSDQSVKTLASKGRLDPLQESMDYNNVYTEILDKVNQYFSLYDMNKFRHKLNLGFSKFISFPNHNVLDGNTKVSSGKREILQEILNGLHANPTFGNLKDVGITTPFGQLQQPNGILLSDETKIRYQSPTGLFERTVSLKDL

SEQ ID NO: 17 - protein, 1334 aa, Listeria innocua
MKKPYTIGLDIGTNSVGWAVLTDQYDLVKRKMKIAGDSEKKQIKKNFWGVRLFDEGQTAADRRMARTARRRIERRRNRISYLQGIFAEEMSKTDANFFCRLSDSFYVDNEKRNSRHPFFATIEEEVEYHKNYPTIYHLREELVNSSEKADLRLVYLALAHIIKYRGNFLIEGALDTQNTSVDGIYKQFIQTYNQVFASGIEDGSLKKLEDNKDVAKILVEKVTRKEKLERILKLYPGEKSAGMFAQFISLIVGSKGNFQKPFDLIEKSDIECAKDSYEEDLESLLALIGDEYAELFVAAKNAYSAVVLSSIITVAETETNAKLSASMIERFDTHEEDLGELKAFIKLHLPKHYEEIFSNTEKHGYAGYIDGKTKQADFYKYMKMTLENIEGADYFIAKIEKENFLRKQRTFDNGAIPHQLHLEELEAILHQQAKYYPFLKENYDKIKSLVTFRIPYFVGPLANGQSEFAWLTRKADGEIRPWNIEEKVDFGKSAVDFIEKMTNKDTYLPKENVLPKHSLCYQKYLVYNELTKVRYINDQGKTSYFSGQEKEQIFNDLFKQKRKVKKKDLELFLRNMSHVESPTIEGLEDSFNSSYSTYHDLLKVGIKQEILDNPVNTEMLENIVKILTVFEDKRMIKEQLQQFSDVLDGVVLKKLERRHYTGWGRLSAKLLMGIRDKQSHLTILDYLMNDDGLNRNLMQLINDSNLSFKSIIEKEQVTTADKDIQSIVADLAGSPAIKKGILQSLKIVDELVSVMGYPPQTIVVEMARENQTTGKGKNNSRPRYKSLEKAIKEFGSQILKEHPTDNQELRNNRLYLYYLQNGKDMYTGQDLDIHNLSNYDIDHIVPQSFITDNSIDNLVLTSSAGNREKGDDVPPLEIVRKRKVFWEKLYQGNLMSKRKFDYLTKAERGGLTEADKARFIHRQLVETRQITKNVANILHQRFNYEKDDHGNTMKQVRIVTLKSALVSQFRKQFQLYKVRDVNDYHHAHDAYLNGVVANTLLKVYPQLEPEFVYGDYHQFDWFKANKATAKKQFYTNIMLFFAQKDRIIDENGEILWDKKYLDTVKKVMSYRQMNIVKKTEIQKGEFSKATIKPKGNSSKLIPRKTNWDPMKYGGLDSPNMAYAVVIEYAKGKNKLVFEKKIIRVTIMERKAFEKDEKAFLEEQGYRQPKVLAKLPKYTLYECEEGRRRMLASANEAQKGNQQVLPNHLVTLLHHAANCEVSDGKSLDYIESNREMFAELLAHVSEFAKRYTLAEANLNKINQLFEQNKEGDIKAIAQSFVDLMAFNAMGAPASFKFFETTIERKRYNNLKELLNSTIIYQSITGLYESRKRLDD

SEQ ID NO: 18 - protein, 1372 aa, Legionella pneumophila
MESSQILSPIGIDLGGKFTGVCLSHLEAFAELPNHANTKYSVILIDHNNFQLSQAQRRATRHRVRNKKRNQFVKRVALQLFQHILSRDLNAKEETALCHYLNNRGYTYVDTDLDEYIKDETTINLLKELLPSESEHNFIDWFLQKMQSSEFRKILVSKVEEKKDDKELKNAVKNIKNFITGFEKNSVEGHRHRKVYFENIKSDITKDNQLDSIKKKIPSVCLSNLLGHLSNLQWKNLHRYLAKNPKQFDEQTFGNEFLRMLKNFRHLKGSQESLAVRNLIQQLEQSQDYISILEKTPPEITIPPYEARTNTGMEKDQSLLLNPEKLNNLYPNWRNLIPGIIDAHPFLEKDLEHTKLRDRKRIISPSKQDEKRDSYILQRYLDLNKKIDKFKIKKQLSFLGQGKQLPANLIETQKEMETHFNSSLVSVLIQIASAYNKEREDAAQGIWFDNAFSLCELSNINPPRKQKILPLLVGAILSEDFINNKDKWAKFKIFWNTHKIGRTSLKSKCKEIEEARKNSGNAFKIDYEEALNHPEHSNNKALIKIIQTIPDIIQAIQSHLGHNDSQALIYHNPFSLSQLYTILETKRDGFHKNCVAVTCENYWRSQKTEIDPEISYASRLPADSVRPFDGVLARMMQRLAYEIAMAKWEQIKHIPDNSSLLIPIYLEQNRFEFEESFKKIKGSSSDKTLEQAIEKQNIQWEEKFQRIINASMNICPYKGASIGGQGEIDHIYPRSLSKKHFGVIFNSEVNLIYCSSQGNREKKEEHYLLEHLSPLYLKHQFGTDNVSDIKNFISQNVANIKKYISFHLLTPEQQKAARHALFLDYDDEAFKTITKFLMSQQKARVNGTQKFLGKQIMEFLSTLADSKQLQLEFSIKQITAEEVHDHRELLSKQEPKLVKSRQQSFPSHAIDATLTMSIGLKEFPQFSQELDNSWFINHLMPDEVHLNPVRSKEKYNKPNISSTPLFKDSLYAERFIPVWVKGETFAIGFSEKDLFEIKPSNKEKLFTLLKTYSTKNPGESLQELQAKSKAKWLYFPINKTLALEFLHHYFHKEIVTPDDTTVCHFINSLRYYTKKESITVKILKEPMPVLSVKFESSKKNVLGSFKHTIALPATKDWERLFNHPNFLALKANPAPNPKEFNEFIRKYFLSDNNPNSDIPNNGHNIKPQKHKAVRKVFSLPVIPGNAGTMMRIRRKDNKGQPLYQLQTIDDTPSMGIQINEDRLVKQEVLMDAYKTRNLSTIDGINNSEGQAYATFDNWLTLPVSTFKPEIIKLEMKPHSKTRRYIRITQSLADFIKTIDEALMIKPSDSIDDPLNMPNEIVCKNKLFGNELKPRDGKMKIVSTGKIVTYEFESDSTPQWIQTLYVTQLKKQP

SEQ ID NO: 19 - protein, 1082 aa, Neisseria lactamica
MAAFKPNPMNYILGLDIGIASVGWAMVEVDEEENPIRLIDLGVRVFERAEVPKTGDSLAMARRLARSVRRLTRRRAHRLLRARRLLKREGVLQDADFDENGLVKSLPNTPWQLRAAALDRKLTCLEWSAVLLHLVKHRGYLSQRKNEGETADKELGALLKGVADNAHALQTGDFRTPAELALNKFEKESGHIRNQRGDYSHTFSRKDLQAELNLLFEKQKEFGNPHVSDGLKEDIETLLMAQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSTELQDEIGTAFSLFKTDKDITGRLKDRVQPEILEALLKHISFDKFVQISLKALRRIVPLMEQGKRYDEACAEIYGDHYCKKNAEEKIYLPPIPADEIRNPVVLRALSQARKVINCVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEEGFKERNLNDTRYVNRFLCQFVADHILLTGKGKRRVFASNGQITNLLRGFWGLRKVRTENDRHHALDAVVVACSTVAMQQKITRFVRYKEMNAFDGKTIDKETGEVLHQKAHFPQPWEFFAQEVMIRVFGKPDGKPEFEEADTPEKLRTLLAEKLSSRPEAVHEYVTPLFVSRAPNRKMSGQGHMETVKSAKRLDEGISVLRVPLTQLKLKGLEKMVNREREPKLYDALKAQLETHKDDPAKAFAEPFYKYDKAGSRTQQVKAVRIEQVQKTGVWVRNHNGIADNATMVRVDVFEKGGKYYLVPIYSWQVAKGILPDRAVVAFKDEEDWTVMDDSFEFRFVLYANDLIKLTAKKNEFLGYFVSLNRATGAIDIRTHDTDSTKGKNGIFQSVGVKTALSFQKNQIDELGKEIRPCRLKKRPPVR

SEQ ID NO: 20 - protein, 1082 aa, Neisseria meningitidis; identical to SEQ ID NO: 4

SEQ ID NO: 21 - protein, 1187 aa, Bifidobacterium longum
MLSRQLLGASHLARPVSYSYNVQDNDVHCSYGERCFMRGKRYRIGIDVGLNSVGLAAVEVSDENSPVRLLNAQSVIHDGGVDPQKNKEAITRKNMSGVARRTRRMRRRKRERLHKLDMLLGKFGYPVIEPESLDKPFEEWHVRAELATRYIEDDELRRESISIALRHMARHRGWRNPYRQVDSLISDNPYSKQYGELKEKAKAYNDDATAAEEESTPAQLVVAMLDAGYAEAPRLRWRTGSKKPDAEGYLPVRLMQEDNANELKQIFRVQRVPADEWKPLFRSVFYAVSPKGSAEQRVGQDPLAPEQARALKASLAFQEYRIANVITNLRIKDASAELRKLTVDEKQSIYDQLVSPSSEDITWSDLCDFLGFKRSQLKGVGSLTEDGEERISSRPPRLTSVQRIYESDNKIRKPLVAWWKSASDNEHEAMIRLLSNTVDIDKVREDVAYASAIEFIDGLDDDALTKLDSVDLPSGRAAYSVETLQKLTRQMLTTDDDLHEARKTLFNVTDSWRPPADPIGEPLGNPSVDRVLKNVNRYLMNCQQRWGNPVSVNIEHVRSSFSSVAFARKDKREYEKNNEKRSIFRSSLSEQLRADEQMEKVRESDLRRLEAIQRQNGQCLYCGRTITFRTCEMDHIVPRKGVGSTNTRTNFAAVCAECNRMKSNTPFAIWARSEDAQTRGVSLAEAKKRVTMFTFNPKSYAPREVKAFKQAVIARLQQTEDDAAIDNRSIESVAWMADELHRRIDWYFNAKQYVNSASIDDAEAETMKTTVSVFQGRVTASARRAAGIEGKIHFIGQQSKTRLDRRHHAVDASVIAMMNTAAAQTLMERESLRESQRLIGLMPGERSWKEYPYEGTSRYESFHLWLDNMDVLLELLNDALDNDRIAVMQSQRYVLGNSIAHDATIHPLEKVPLGSAMSADLIRRASTPALWCALTRLPDYDEKEGLPEDSHREIRVHDTRYSADDEMGFFASQAAQIAVQEGSADIGSAIHHARVYRCWKTNAKGVRKYFYGMIRVFQTDLLRACHDDLFTVPLPPQSISMRYGEPRVVQALQSGNAQYLGSLVVGDEIEMDFSSLDVDGQIGEYLQFFSQFSGGNLAWKHWVVDGFFNQTQLRIRPRYLAAEGLAKAFSDDVVPDGVQKIVTKQGWLPPVNTASKTAVRIVRRNAFGEPRLSSAHHMPCSWQWRHE

SEQ ID NO: 22 - protein, 1101 aa, Francisella novicida
MSRSLTFSFDIGYASIGWAVIASASHDDADPSVCGCGTVLFPKDDCQAFKRREYRRLRRNIRSRRVRIERIGRLLVQAQIITPEMKETSGHPAPFYLASEALKGHRTLAPIELWHVLRWYAHNRGYDNNASWSNSLSEDGGNGEDTERVKHAQDLMDKHGTATMAETICRELKLEEGKADAPMEVSTPAYKNLNTAFPRLIVEKEVRRILELSAPLIPGLTAEIIELIAQHHPLTTEQRGVLLQHGIKLARRYRGSLLFGQLIPRFDNRIISRCPVTWAQVYEAELKKGNSEQSARERAEKLSKVPTANCPEFYEYRMARILCNIRADGEPLSAEIRRELMNQARQEGKLTKASLEKAISSRLGKETETNVSNYFTLHPDSEEALYLNPAVEVLQRSGIGQILSPSVYRIAANRLRRGKSVTPNYLLNLLKSRGESGEALEKKIEKESKKKEADYADTPLKPKYATGRAPYARTVLKKVVEEILDGEDPTRPARGEAHPDGELKAHDGCLYCLLDTDSSVNQHQKERRLDTMTNNHLVRHRMLILDRLLKDLIQDFADGQKDRISRVCVEVGKELTTFSAMDSKKIQRELTLRQKSHTDAVNRLKRKLPGKALSANLIRKCRIAMDMNWTCPFTGATYGDHELENLELEHIVPHSFRQSNALSSLVLTWPGVNRMKGQRTGYDFVEQEQENPVPDKPNLHICSLNNYRELVEKLDDKKGHEDDRRRKKKRKALLMVRGLSHKHQSQNHEAMKEIGMTEGMMTQSSHLMKLACKSIKTSLPDAHIDMIPGAVTAEVRKAWDVFGVFKELCPEAADPDSGKILKENLRSLTHLHHALDACVLGLIPYIIPAHHNGLLRRVLAMRRIPEKLIPQVRPVANQRHYVLNDDGRMMLRDLSASLKENIREQLMEQRVIQHVPADMGGALLKETMQRVLSVDGSGEDAMVSLSKKKDGKKEKNQVKASKLVGVFPEGPSKLKALKAAIEIDGNYGVALDPKPVVIRHIKVFKRIMALKEQNGGKPVRILKKGMLIHLTSSKDPKHAGVWRIESIQDSKGGVKLDLQRAHCAVPKNKTHECNWREVDLISLLKKYQMKRYPTSYTGTPR

SEQ ID NO: 23 - protein, 1498 aa, Odoribacter laneus
METTLGIDLGTNSIGLALVDQEEHQILYSGVRIFPEGINKDTIGLGEKEESRNATRRAKRQMRRQYFRKKLRKAKLLELLIAYDMCPLKPEDVRRWKNWDKQQKSTVRQFPDTPAFREWLKQNPYELRKQAVTEDVTRPELGRILYQMIQRRGFLSSRKGKEEGKIFTGKDRMVGIDETRKNLQKQTLGAYLYDIAPKNGEKYRFRTERVRARYTLRDMYIREFEIIWQRQAGHLGLAHEQATRKKNIFLEGSATNVRNSKLITHLQAKYGRGHVLIEDTRITVTFQLPLKEVLGGKIEIEEEQLKFKSNESVLFWQRPLRSQKSLLSKCVFEGRNFYDPVHQKWIIAGPTPAPLSHPEFEEFRAYQFINNIIYGKNEHLTAIQREAVFELMCTESKDFNFEKIPKHLKLFEKFNFDDTTKVPACTTISQLRKLFPHPVWEEKREEIWHCFYFYDDNTLLFEKLQKDYALQTNDLEKIKKIRLSESYGNVSLKAIRRINPYLKKGYAYSTAVLLGGIRNSFGKRFEYFKEYEPEIEKAVCRILKEKNAEGEVIRKIKDYLVHNRFGFAKNDRAFQKLYHHSQAITTQAQKERLPETGNLRNPIVQQGLNELRRTVNKLLATCREKYGPSFKFDHIHVEMGRELRSSKTEREKQSRQIRENEKKNEAAKVKLAEYGLKAYRDNIQKYLLYKEIEEKGGTVCCPYTGKTLNISHTLGSDNSVQIEHIIPYSISLDDSLANKTLCDATFNREKGELTPYDFYQKDPSPEKWGASSWEEIEDRAFRLLPYAKAQRFIRRKPQESNEFISRQLNDTRYISKKAVEYLSAICSDVKAFPGQLTAELRHLWGLNNILQSAPDITFPLPVSATENHREYYVITNEQNEVIRLFPKQGETPRTEKGELLLTGEVERKVFRCKGMQEFQTDVSDGKYWRRIKLSSSVTWSPLFAPKPISADGQIVLKGRIEKGVFVCNQLKQKLKTGLPDGSYWISLPVISQTFKEGESVNNSKLTSQQVQLFGRVREGIFRCHNYQCPASGADGNFWCTLDTDTAQPAFTPIKNAPPGVGGGQIILTGDVDDKGIFHADDDLHYELPASLPKGKYYGIFTVESCDPTLIPIELSAPKTSKGENLIEGNIWVDEHTGEVRFDPKKNREDQRHHAIDAIVIALSSQSLFQRLSTYNARRENKKRGLDSTEHFPSPWPGFAQDVRQSVVPLLVSYKQNPKTLCKISKTLYKDGKKIHSCGNAVRGQLHKETVYGQRTAPGATEKSYHIRKDIRELKTSKHIGKVVDITIRQMLLKHLQENYHIDITQEFNIPSNAFFKEGVYRIFLPNKHGEPVPIKKIRMKEELGNAERLKDNINQYVNPRNNHHVMIYQDADGNLKEEIVSFWSVIERQNQGQPIYQLPREGRNIVSILQINDTFLIGLKEEEPEVYRNDLSTLSKHLYRVQKLSGMYYTFRHHLASTLNNEREEFRIQSLEAWKRANPVKVQIDEIGRITFLNGPLC

SEQ ID NO: 24 - protein, 509 aa, Homo sapiens
MADAEVIILPKKHKKKKERKSLPEEDVAEIQHAEEFLIKPESKVAKLDTSQWPLLLKNFDKLNVRTTHYTPLACGSNPLKREIGDYIRTGFINLDKPSNPSSHEVVAWIRRILRVEKTGHSGTLDPKVTGCLIVCIERATRLVKSQQSAGKEYVGIVRLHNAIEGGTQLSRALETLTGALFQRPPLIAAVKRQLRVRTIYESKMIEYDPERRLGIFWVSCEAGTYIRTLCVHLGLLLGVGGQMQELRRVRSGVMSEKDHMVTMHDVLDAQWLYDNHKDESYLRRVVYPLEKLLTSHKRLVMKDSAVNAICYGAKIMLPGVLRYEDGIEVNQEIVVITTKGEAICMAIALMTTAVISTCDHGIVAKIKRVIMERDTYPRKWGLGPKASQKKLMIKQGLLDKHGKPTDSTPATWKQDESAKKEVVAEVVKAPQVVAEAAKTAKRKRESESESDETPPAAPQLIKKEKKKSKKDKKAKAGLESGAEPGDGDSDTTKKKKKKKKAKEVELVSE

SEQ ID NO: 25 - DNA, 2593 bp, Homo sapiens
gtactggccgagccagcaaatcgcattgcgcagacgaccagcgggcgcctcggattccgcccccgggatggccccgcctcctcccgccccgcggcaaggcacgcacagggcagtgcgcgggtgggtgggtcctagcagcgcggcctgacgggaccaaggcggcgggagtctgcggtcgttccctcggctgtggaccgggcggcacgcacgcggtgcagggtaacatggcggatgcggaagtaattattttgccaaagaaacataagaagaaaaaggagcggaagtcattgccagaagaagatgtagccgaaatacaacacgctgaagaatttcttatcaaacctgaatccaaagttgctaagttggacacgtctcagtggccccttttgctaaagaattttgataagctgaatgtaaggacaacacactatacacctcttgcatgtggttcaaatcctctgaagagagagattggggactatatcaggacaggtttcattaatcttgacaagccctctaacccctcttcccatgaggtggtagcctggattcgacggatacttcgggtggagaagacagggcacagtggtactctggatcccaaggtgactggttgtttaatcgtgtgcatagaacgagccactcgcttggtgaagtcacaacagagtgcaggcaaagagtatgtggggattgtccggctgcacaatgctattgaaggggggacccagctttctagggccctagaaactctgacaggtgccttattccagcgacccccacttattgctgcagtaaagaggcagctccgagtgaggaccatctacgagagcaaaatgattgaatacgatcctgaaagaagattaggaatcttttgggtgagttgtgaggctggcacctacattcggacattatgtgtgcaccttggtttgttattgggagttggtggtcagatgcaggagcttcggagggttcgttctggagtcatgagtgaaaaggaccacatggtgacaatgcatgatgtgcttgatgctcagtggctgtatgataaccacaaggatgagagttacctgcggcgagttgtttaccctttggaaaagctgttgacatctcataaacggctggttatgaaagacagtgcagtaaatgccatctgctatggggccaagattatgcttccaggtgttcttcgatatgaggacggcattgaggtcaatcaggagattgtggttatcaccaccaaaggagaagcaatctgcatggctattgcattaatgaccacagcggtcatctctacctgcgaccatggtatagtagccaagatcaagagagtgatcatggagagagacacttaccctcggaagtggggtttaggtccaaaggcaagtcagaagaagctgatgatcaagcagggccttctggacaagcatgggaagcccacagacagcacacctgccacctggaagcaggatgagtctgccaaaaaagaggtggttgctgaagtggtaaaagccccgcaggtagttgccgaagcagcaaaaactgcgaagcggaagcgagagagtgagagtgaaagtgacgagactcctccagcagctcctcagttgatcaagaaggaaaagaagaagagtaagaaggacaagaaggccaaagctggtctggagagcggggccgagcctggagatggggacagtgataccaccaagaagaagaagaagaagaagaaagcaaaagaggtagaattggtttctgagtagtgaaggccacttgaagctggaggagaaactaaagccttattgagaaaacatgttatagatccttttgttgctgagagagtggaacataggtcctagacagggtgaagagttctggcacattttagctgctactttgagacctcggtgatgttacctggtgtggtcatcccatcttgtcctgttttaaggatatgggtggtgaaagatgaaagaggcagagtttatcccaatgacttctctgtttgagttgggaagcctcaccttcagacccagtaactgtccgcagctgtctgctagtggttgtcttaacatcgtagtcctagtttgcattttttaaatcccctctgtttaaaaggtttgtaaaacaaaaacaaaaaactaagtctgctcagtgaaatgctgtagaaccctaaataagtggtagaagagtgtcactgaattttgtctctgaattcagtataactgagttttgtccatgctggtgtctgggttataggcctgatgggcctggtagttttccatcttgttctggcctagaggtcagtcctttgcacttcctcaaagcttgtgtacagtgctcacctaaatccatctgactacttgttcctgtgccctcttgttttaggcctcgtttacttttaaaaaatgaaattgttcattgctgggagaagaatgttgtaatttttacttattaaagtcaacttgttaagttttttatgtattcctgttgggttttcttgttgatctcatgctagcagagcaaaaattgtaaaatattttgattaaaaatctagggacctttatgtcctatttgaaatgtgaaaaaaaaaaaaaaaaaa

SEQ ID NO: 26 - protein, 399 aa, Homo sapiens
MAGNAEPPPAGAACPQDRRSCSGRAGGDRVWEDGEHPAKKLKSGGDEERREKPPKRKIVLLMAYSGKGYHGMQRNVGSSQFKTIEDDLVSALVRSGCIPENHGEDMRKMSFQRCARTDKGVSAAGQVVSLKVWLIDDILEKINSHLPSHIRILGLKRVTGGFNSKNRCDARTYCYLLPTFAFAHKDRDVQDETYRLSAETLQQVNRLLACYKGTHNFHNFTSQKGPQDPSACRYILEMYCEEPFVREGLEFAVIRVKGQSFMMHQIRKMVGLVVAIVKGYAPESVLERSWGTEKVDVPKAPGLGLVLERVHFEKYNQRFGNDGLHEPLDWAQEEGKVAAFKEEHIYPTIIGTERDERSMAQWLSTLPIHNFSATALTAGGTGAKVPSPLEGSEGDGDTD

SEQ ID NO: 27 - DNA, 1637 bp, Homo sapiens
cccacgtggtccggctccggctcagtcagccgcgtcgcgaatggggcaggagcgagcctctctggtcccgacgcgggtggcccgggtctcctcgactcctgaggaaagcccaccgggcggggcgggaggtgaagaggctggggaagtcagagctcgccgcgcatggccgggaacgcggagccgccgcccgccggagccgcatgcccccaggaccggaggtcctgcagcggccgggccgggggcgaccgcgtctgggaggacggagaacatccggcgaagaagctcaagagcggtggcgacgaggagcggcgcgagaagccgcccaagcggaagatcgtgctgctcatggcctattcgggcaagggctaccacggcatgcagaggaatgtcgggtcctcacaattcaaaacaattgaagatgacttggtgtccgccctcgtccggtcaggctgtattcctgaaaatcatggtgaggacatgaggaaaatgtccttccagcgctgcgcccggacagacaagggtgtgtccgcagccggccaggtggtatccctgaaggtgtggctgattgacgacattctagaaaagatcaacagccaccttccctctcacattcggattctgggactgaagcgggtcacgggcgggtttaactccaagaacagatgtgatgccaggacctattgctacctgctgcccacgtttgcctttgcgcacaaggaccgggacgttcaggatgagacctaccgcctgagcgccgagacgctgcagcaggtcaacaggctcctggcctgctacaagggcacgcacaacttccacaatttcacctcgcagaaggggccgcaggatcccagtgcctgccgctacatcctggagatgtactgcgaggaaccctttgtgcgggagggcctggagtttgcggtgatcagggtgaagggccagagcttcatgatgcatcagatccggaagatggtcggcctggtggtggccattgtgaagggttatgcccctgagagcgtgctggagcgcagctggggcacagagaaggtggacgtgcccaaggcgcccggactcggcctggtcctggagagggtgcacttcgagaagtacaaccagcgctttggcaacgatgggctgcatgagccgctggactgggcgcaggaggaaggaaaggtcgcagccttcaaggaggagcacatctaccccaccatcatcggcaccgagcgggacgaacgctccatggcccagtggctgagcaccttgcccatccacaacttcagtgccaccgctctcacggcaggtggcacgggcgccaaggtgcccagtcccctggaaggcagtgaaggggacggagacactgactgaggcgatgggagctgcccaccagagtgcctctgagcagctcacagtgtgtgcccagatgtgccacccctgtgggcagcaagaagctgggatcgctgcagccatgttttcccggccatgccggcgttgtaacctcaggaccttcccttgtaggaacagcctttctcgaatctgttttcagctcttgcattgcatagatgaacctcagcatgtaaagaactatttttttaaagaagtgattttcttattaaacaagtacaaattttgcttagtcaatc

SEQ ID NO: 28 - protein, 481 aa, Homo sapiens
MADNDTDRNQTEKLLKRVRELEQEVQRLKKEQAKNKEDSNIRENSAGAGKTKRAFDFSAHGRRHVALRIAYMGWGYQGFASQENTNNTIEEKLFEALTKTRLVESRQTSNYHRCGRTDKGVSAFGQVISLDLRSQFPRGRDSEDFNVKEEANAAAEEIRYTHILNRVLPPDIRILAWAPVEPSFSARFSCLERTYRYFFPRADLDIVTMDYAAQKYVGTHDFRNLCKMDVANGVINFQRTILSAQVQLVGQSPGEGRWQEPFQLCQFEVTGQAFLYHQVRCMMAILFLIGQGMEKPEIIDELLNIEKNPQKPQYSMAVEFPLVLYDCKFENVKWIYDQEAQEFNITHLQQLWANHAVKTHMLYSMLQGLDTVPVPCGIGPKMDGMTEWGNVKPSVIKQTSAFVEGVKMRTYKPLMDRPKCQGLESRIQHFVRRGRIEHPHLFHEEETKAKRDCNDTLEEENTNLETPTKRVCVDTEIKSII

SEQ ID NO: 29 - DNA, 1862 bp, Homo sapiens
gcacagtgacagcttcctttctcggaaacgcggcgcggccggctgccggaaaacagggcagacctgtatggttcgtttattcctggggttgtcatatcatggctgataatgacacagacagaaaccagactgagaagctcctaaaaagagtacgagaactggagcaagaggtgcaaagacttaaaaaggaacaggccaaaaataaggaggactcaaacattagagaaaattcagcaggagctggaaaaactaagcgtgcatttgatttcagtgctcatggccgaagacacgtagccctaagaatagcctatatgggctggggataccagggctttgctagtcaggaaaacacaaataataccattgaagagaaactgtttgaagctctaaccaagactcgactagtagaaagcagacagacatccaactatcaccgatgtgggagaacagataaaggagttagtgcctttggacaggtgatctcacttgaccttcgctctcagtttccaaggggcagggattccgaggactttaatgtaaaagaggaggctaatgctgctgctgaagagatccgttatacccacattctcaatcgggtactccctccagacatccgtatattggcctgggcccctgtagaaccaagcttcagtgctaggttcagctgccttgagcggacttaccgctattttttccctcgtgctgatttagatattgtaaccatggattatgcagctcagaagtatgttggcacccatgatttcaggaacttgtgtaaaatggatgtagccaacggtgtgattaattttcagaggactattctatctgctcaagtacagctagtgggccagagcccaggtgaggggagatggcaagaacctttccagttatgtcagtttgaagtgactggccaggcattcctttatcatcaagtccgatgtatgatggctatcctctttctgattggccaaggaatggagaagccagagattattgatgagctgctgaatatagagaaaaatccccaaaagcctcaatatagtatggctgtagaatttcctctagtcttatatgactgtaagtttgaaaatgtcaagtggatctatgaccaggaggctcaggagttcaatattacccacctacaacaactgtgggctaatcatgctgtcaaaactcacatgttgtatagtatgctacaaggactggacactgttccagtaccctgtggaataggaccaaagatggatggaatgacagaatggggaaatgttaagccctctgtcataaagcagaccagtgcctttgtagaaggagtgaagatgcgcacatataagcccctcatggaccgtcctaaatgccaaggactggaatcccggatccagcattttgtacgtaggggacgaattgagcacccacatttattccatgaggaagaaacaaaagccaaaagggactgtaatgacacactagaggaagagaatactaatttggagacaccaacgaagagggtctgtgttgacacagaaattaaaagcatcatttaaccatagacaatttgccaggatctaggaaccacctaatggtaggtggacagaaaaggaaaaaaaaaaaaatttacttgcaagtactaggaattcagatgatcagctcttaaaagaaaaaaaaaagcaaaaagactaaagccctattaaggaagttattgctttaataagaaatttcaaatattctcttatcccggtccaaaaggattaagcgattaaagaacgtaaaatggagatgtatttacatacacctggaaacctgtgccttgtattcaaattcattaaagcctaatcctgcaagtaaaaaaaaaaaaaaaaaaa

SEQ ID NO: 30 - protein, 667 aa, Homo sapiens
MEMTEMTGVSLKRGALVVEDNDSGVPVEETKKQKLSECSLTKGQDGLQNDFLSISEDVPRPPDTVSTGKGGKNSEAQLEDEEEEEEDGLSEECEEEESESFADMMKHGLTEADVGITKFVSSHQGFSGILKERYSDFVVHEIGKDGRISHLNDLSIPVDEEDPSEDIFTVLTAEEKQRLEELQLFKNKETSVAIEVIEDTKEKRTIIHQAIKSLFPGLETKTEDREGKKYIVAYHAAGKKALAKVRTAADPRKHSWPKSRGSYCHFVLYKENKDTMDAINVLSKYLRVKPNIFSYMGTKDKRAITVQEIAVLKITAQRLAHLNKCLMNFKLGNFSYQKNPLKLGELQGNHFTVVLRNITGTDDQVQQAMNSLKEIGFINYYGMQRFGTTAVPTYQVGRAILQNSWTEVMDLILKPRSGAEKGYLVKCREEWAKTKDPTAALRKLPVKRCVEGQLLRGLSKYGMKNIVSAFGIIPRNNRLMYIHSYQSYVWNNMVSKRIEDYGLKPVPGDLVLKGATATYIEEDDVNNYSIHDVVMPLPGFDVIYPKHKIQEAYREMLTADNLDIDNMRHKIRDYSLSGAYRKIIIRPQNVSWEVVAYDDPKIPLFNTDVDNLEGKTPPVFASEGKYRALKMDFSLPPSTYATMAIREVLKMDTSIKNQTQLNTTWLR

SEQ ID NO: 31 - DNA, 3316 bp, Homo sapiens
ccttaaagatggagatgacagaaatgactggtgtgtcgctgaaacgtggggcactggttgtcgaagataatgacagtggagtcccagttgaagagacaaaaaaacagaagctgtcggaatgcagtctaaccaaaggtcaagatgggctacagaatgactttctgtccatcagtgaagacgtgcctcggcctcctgacactgtcagtactgggaaaggtggaaagaattctgaggctcagttggaagatgaggaagaagaggaggaagatggactttcagaggagtgcgaggaggaggaatcagagagttttgcagacatgatgaagcatggactcactgaggctgacgtaggcatcaccaagtttgtgagttctcatcaagggttctcgggaatcttaaaagaaagatactccgacttcgttgttcatgaaataggaaaagatggacggatcagccatttgaatgacttgtccattccagtggatgaggaggacccttcagaagacatatttacagttttgacagctgaagaaaagcagcgattggaagagctccagctgttcaaaaataaggaaaccagtgttgccattgaggttatcgaggacaccaaagagaaaagaaccatcatccatcaggctatcaaatctctgtttccaggattagagacaaaaacagaggatagggaggggaagaaatacattgtagcctaccacgcagctgggaaaaaggctttggcaaaggtcagaactgcagcagatccaagaaaacattcttggccaaaatctaggggaagttactgccacttcgtactatataaggaaaacaaagacaccatggatgctattaatgtactctccaaatacttaagagtcaagccaaatatattctcctacatgggaaccaaagataaaagggctataacagttcaagaaattgctgttctcaaaataactgcacaaagacttgcccacctgaataagtgcttgatgaactttaagctagggaatttcagctatcaaaaaaacccactgaaattgggagagcttcaaggaaaccacttcactgttgttctcagaaatataacaggaactgatgaccaagtacagcaagctatgaactctctcaaggagattggatttattaactactatggaatgcaaagatttggaaccacagctgtccctacgtatcaggttggaagagctatactacaaaattcctggacagaagtcatggatttaatattgaaaccccgctctggagctgaaaagggctacttggttaaatgcagagaagaatgggcaaagaccaaagacccaactgctgccctcagaaaactacctgtcaaaaggtgtgtggaagggcagctgcttcgaggactttcaaaatatggaatgaagaatatagtctctgcatttggcataatacccagaaataatcgcttaatgtatattcatagctaccaaagctatgtgtggaataacatggtaagcaagaggatagaagactatggactaaaacctgttccaggggacctcgttctcaaaggagccacagccacctatattgaggaagatgatgttaataattactctatccatgatgtggtaatgcccttgcctggtttcgatgttatctacccaaagcataaaattcaagaagcctacagggaaatgctcacagctgacaatcttgatattgacaacatgagacacaaaattcgagattattccttgtcaggggcctaccgaaagatcattattcgtcctcagaatgttagctgggaagtcgttgcatatgatgatcccaaaattccacttttcaacacagatgtggacaacctagaagggaagacaccaccagtttttgcttctgaaggcaaatacagggctctgaaaatggatttttctctacccccttctacttacgccaccatggccattcgagaagtgctaaaaatggataccagtatcaagaaccagacgcagctgaatacaacctggcttcgctgagcagtaccttgtccacagattagaaaacgtacacaagtgtttgcttcctggctccctgtgcatttttgtcttagttcagactcatatatggatttcaaatctttgtaataaaaattatttgtatttttaagtttttattagcttaaagaaataatttgcaatatttgtacatgtacacaaatcctgaggttcttaattttagctcagaatataaattagtcaaaatacacttcaggtgcttaaatcagagtaaaatgtcagctttacaataataaaaaaaggactttggtttaaagtagcaggtttaggttttgctacattctcaaaagacagcaggagtatttgacacatctgtgatggagtatacaacaatgcattttaagagcaaatgcaacaaaacaaatctggactatggataaataatttgagagctgccacccacaaatataaatacagtactcatgctgactgaaataataagacatctacaaatttataaacaaaaagtgattgtcattatcctgcttatgtactagattcaggcaagcattatagactttttggttgcggtggcttttgcatttatattatcaatgccttgcaggaacgttgcattgataggcccattttatttttttattttttttttcgagacaggatctcactctgtagcacaggctggattgcagtgcaatcctgcaattctcaatcttgcactgcagcctcgacctcccaggctccagtgactctcccacctcagcctcctaagtagctgggagtacaggcgcgcaccaccacgcctagctgatttttgtatttttttgtagagacgggggtttggccatgttgccgaggctaactcctgggattacaggcatgagctgtgctggccgggtttttttttcttgatgtaaacgtgtacagctgttttattagttaaggtctaatttttactctaggtgccttttatgttcagaactctttccactggactggtatttgctcaaaaataaataatggtagagaagaaaactataaaaatggacaaggctttcttctatcagtagcgtttaccctttgtcaccagtggctttggtatttccatgtctggcattgcataaacttctctggtgtgaaaggataaatatgcctttctaaagttgtatatcaaaattgtatcaatttttattttctatgatttctagaaacaaatgtaataaatatttttaaaatctcctttctactggttatgtaaataaatcaaataaatatatcaaaatgagtgcagaaaaaaaaaaaaaaaa